SEQUENCE LISTING (24 sequences)

SEQ ID NO: 1 - DNA, 23 bp, synthetic construct
  source 1..23: mol_type = other DNA
gaaccagatgcacattgatgaag

SEQ ID NO: 2 - DNA, 22 bp, synthetic construct
  source 1..22: mol_type = other DNA
tgcccacatattctcctttgag

SEQ ID NO: 3 - DNA, 17 bp, synthetic construct
  source 1..17: mol_type = other DNA
caggccccattgttccc

SEQ ID NO: 4 - DNA, 20 bp, synthetic construct
  source 1..20: mol_type = other DNA
attgctctggtcacttctcc

SEQ ID NO: 5 - DNA, 19 bp, synthetic construct
  source 1..19: mol_type = other DNA
tgaccttctcctgccacta

SEQ ID NO: 6 - DNA, 21 bp, synthetic construct
  source 1..21: mol_type = other DNA
aagcgaaatcctcagtctgac

SEQ ID NO: 7 - DNA, 21 bp, synthetic construct
  source 1..21: mol_type = other DNA
atggcaaagtgaatgacaagc

SEQ ID NO: 8 - DNA, 21 bp, synthetic construct
  source 1..21: mol_type = other DNA
aggcgaagttaatggaagctc

SEQ ID NO: 9 - DNA, 20 bp, synthetic construct
  source 1..20: mol_type = other DNA
actcacttcaggaagcaacc

SEQ ID NO: 10 - DNA, 23 bp, synthetic construct
  source 1..23: mol_type = other DNA
gattgactgaatgctgattcctc

SEQ ID NO: 11 - DNA, 22 bp, synthetic construct
  source 1..22: mol_type = other DNA
ctcagatcattgtcacagtcgt

SEQ ID NO: 12 - DNA, 21 bp, synthetic construct
  source 1..21: mol_type = other DNA
gtcgtcaggaagaagagaacc

SEQ ID NO: 13 - DNA, 21 bp, synthetic construct
  source 1..21: mol_type = other DNA
gtgccatactcatgaccatcg

SEQ ID NO: 14 - DNA, 19 bp, synthetic construct
  source 1..19: mol_type = other DNA
ggccacaaagccaaagatg

SEQ ID NO: 15 - DNA, 21 bp, synthetic construct
  source 1..21: mol_type = other DNA
tccatcctctccatgatccac

SEQ ID NO: 16 - DNA, 25 bp, synthetic construct
  source 1..25: mol_type = other DNA
gttgaagaagctgaagaagttgatg

SEQ ID NO: 17 - DNA, 20 bp, synthetic construct
  source 1..20: mol_type = other DNA
gccatgtccagttacctgtt

SEQ ID NO: 18 - DNA, 21 bp, synthetic construct
  source 1..21: mol_type = other DNA
tttcccttcaagaaccagtcc

SEQ ID NO: 19 - DNA, 20 bp, synthetic construct
  source 1..20: mol_type = other DNA
gcatcctccaaagcactgaa

SEQ ID NO: 20 - DNA, 21 bp, synthetic construct
  source 1..21: mol_type = other DNA
caagtgcatagcaaagcttgt

SEQ ID NO: 21 - DNA, 18 bp, synthetic construct
  source 1..18: mol_type = other DNA
catggcagacgagctgag

SEQ ID NO: 22 - DNA, 20 bp, synthetic construct
  source 1..20: mol_type = other DNA
aaactgtgtgtcccttctgg

SEQ ID NO: 23 - DNA, 19 bp, synthetic construct
  source 1..19: mol_type = other DNA
tggacacctgcatcaagac

SEQ ID NO: 24 - DNA, 22 bp, synthetic construct
  source 1..22: mol_type = other DNA
ggatcttcctcttgaactcctc

(19) United States
(12) Patent Application Publication
(10) Pub. No.: US 2025/0376662 A1
(43) Pub. Date: Dec. 11, 2025

(54) FORWARD PROGRAMMED BLOOD-BRAIN BARRIER MODEL

(71) Applicant: Wisconsin Alumni Research Foundation, Madison, WI (US)

(72) Inventors: Eric Shusta, Madison, WI (US); Sean Palecek, Verona, WI (US); Sarah Boutom, Madison, WI (US); Yunfeng Ding, Madison, WI (US); Soniya Tamhankar, Madison, WI (US)

(21) Appl. No.: 19/232,516

(22) Filed: Jun. 9, 2025

Related U.S. Application Data
(60) Provisional application No. 63/657,363, filed on Jun. 7, 2024.

(51) Int. Cl. C12N 5/071; C12N 15/86

(52) U.S. Cl. CPC C12N 5/069; C12N 15/86; C12N 2501/415; C12N 2503/02; C12N 2506/45; C12N 2510/00; C12N 2740/15043

(57) ABSTRACT

The present invention provides in vitro methods for producing an endothelial cell with blood-brain barrier (BBB)-like properties. The methods include culturing an endothelial cell in a medium comprising a Wnt/β-catenin signaling activator and expressing one or more transcription factors in the endothelial progenitor cells for at least 2 days. The one or more transcription factors are selected from DACH1, DACH2, FLI1, FOS, FOXC1, FOXF1, FOXF2, FOXQ1, HES1, JUN, KLF2, KLF4, LEF1, MECOM, NR4A1, NR4A2, PPARD, TBX3, TSC22D1, ZIC2, ZIC3 and combinations thereof.

### CROSS-REFERENCE TO RELATED APPLICATIONS

This application claims priority to U.S. Provisional Application No. 63/657,363 filed on Jun. 7, 2024, the contents of which is incorporated by reference in its entirety.

### STATEMENT REGARDING FEDERALLY SPONSORED RESEARCH

This invention was made with government support under NS109486 and NS107461 awarded by the National Institutes of Health. The government has certain rights in the invention.

### REFERENCE TO AN ELECTRONIC SEQUENCE LISTING

The contents of the electronic sequence listing (96029604695.xml; Size: 22,021 bytes; and Date of Creation: Jun. 6, 2025) is herein incorporated by reference in its entirety.

### BACKGROUND

The blood-brain barrier (BBB) is composed of specialized vascular endothelial cells that maintain brain homeostasis and regulate the passage of blood solutes into the central nervous system (CNS) by restricting both transcellular and paracellular transport. Endothelial cells of the CNS acquire specialized barrier properties in response to extrinsic signals during development. In particular, Wnt signaling from developing neural tissue coordinates multiple aspects of endothelial barrier function. To date, knowledge of this process has been advanced largely using mouse models. While human pluripotent stem cells (hPSCs) offer the opportunity to examine barrier development in a human system, existing protocols do not fully mimic the developmental trajectory or transcriptional characteristics of CNS endothelial cells. As a result, existing BBB models do not accurately model BBB gene expression and function. Accordingly, there remains a need in the art for BBB models that have in vivo-like BBB properties.

### SUMMARY

Described herein, the inventors provide methods for producing an endothelial cell capable of forming a confluent monolayer with BBB-like properties. In some embodiments, the method comprises culturing a endothelial cell in a medium comprising a Wnt/β-catenin signaling activator and gene delivery strategies to overexpress one or more transcription factors, wherein the one or more transcription factors are selected from the group consisting of, DACH1, DACH2, FLI1, FOS, FOXC1, FOXF1, FOXF2, FOXQ1, HES1, JUN, KLF2, KLF4, LEF1, MECOM, NR4A1, NR4A2, PPARD, TBX3, TSC22D1, ZIC2 and ZIC3 and combinations thereof for 2 to 7 days. In some embodiments, the BBB-like properties comprise increased tight junction protein expression, increased transporter protein expression, reduced transcytosis-related protein expression, reduced leukocyte adhesion molecule expression and combinations thereof. In some embodiments, the Wnt/β-catenin signaling activator is CHIR99021. In some embodiments, the endothelial cell is an endothelial progenitor cell differentiated from a pluripotent stem cell. In some embodiments, the one or more transcription factors are overexpressed. In some embodiments, the overexpression of the one or more transcription factors is achieved via transduction with a virus comprising a polynucleotide encoding one or more transcription factors operably linked to a promoter function in the endothelial progenitor cell. In some embodiments, two or more transcription factors are selected from the group consisting of KLF2, KLF4, ZIC2, ZIC3, NR4A1, NR4A2, FOXQ1, FOXF1 and FOXF2 and combinations thereof. In some embodiments, the two or more transcription factors are selected from KLF2, FOXF1, ZIC3, NR4A2, and FOXQ1.

Another aspect of the present disclosure provides a population of endothelial cells with BBB-like properties produced by the methods described herein. In some embodiments, an in vitro BBB model comprising a confluent monolayer of the endothelial cells described herein is provided. In some embodiments the BBB model has BBB-like properties. In some embodiments, the BBB model is an isogenic model. In some embodiments, the endothelial cells are derived from pluripotent stem cells obtained from a subject. In some embodiments, the subject has a brain disease.

Another aspect of the present disclosure proves a method of using the BBB model described herein. In some embodiments, the method comprises contacting the BBB model with a therapeutic agent and testing the ability of the therapeutic agent to cross the BBB. In some embodiments, the therapeutic agent comprises small molecule drugs, biologics, viral vectors, or therapeutic cells.

### BRIEF DESCRIPTION OF THE DRAWINGS

The patent or application file contains at least one drawing executed in color. Copies of this patent or patent application with color drawing(s) will be provided by the Office upon request and payment of the necessary fee.

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1A-1C. Methodological Approach & Single cell transcriptomic analysis revealed BBB-enriched TFs. FIG. 1A. Methodological approach. Several differentiation protocols depend on external cues with known roles in development, and alternative is Direct overexpression of Transcription Factors (TFs) “Forward Programing”. This approach significantly shortens the time for cell conversion, captures phenotypes not stimulated by external cues, isolates it from other developmental events. Step 1: The generation of lentivirus using HEK293T cells; Step 2: Generation of endothelial progenitor cells (EPCs) from hPSCs; Step 3: Transduction of EPCs with control or TF lentivirus. FIG. 1B. Publicly available single cell transcriptomic datasets were analyzed to determine BBB-enriched TFs. A total of 96 TFs were identified as shown in the Venn diagram. 40 TFs were significantly overexpressed in the brain EC or brain capillary EC populations in non-parametric Wilcoxon rank sum test in at least two of the three independent transcriptomics analysis. Statistical significance is defined as adjusted p-value less than 0.05. Using a preliminary qPCR screen, we determined a list of 21 candidate TFs for individual overexpression and bulk transcriptomic analysis in CNS-like ECs. FIG. 1C. RT-qPCR screen on forward programmed CECs with overexpression of a single TF. Forward programmed CECs were prepared by dosing lentiviruses delivering a single TF at MOI=2 for each condition. RNA was extracted five days after lentivirus transduction and RT-qPCR was performed on samples for 10 genes expressed at the BBB (CDH5, PECAM1, CLDN5, OCLN, ABCB1, ABCG2, SLC2A1, SLC7A5, SLC38A5, MFSD2A) and two genes downregulated at the BBB (CAV1, PLVAP). Fold change of gene expression compared to the GFP control is displayed on the graph and also represented by color gradient. For overexpression, color bar for upregulation is capped at 2-fold overexpression for better visualization. N=1 transduction for each TF is performed.

FIG. 2A-2G. Forward programed CECs with single TF overexpression capitulates some BBB-like gene expression. FIG. 2A. IMR90-4 hPSCs were differentiated to endothelial progenitor cells (EPCs). CD31+ EPCs were sorted using MACS. Lentivirus for TF overexpression was dosed on CD31+ EPCs and the population was treated with CHIR99021 for five days before analysis. The resultant cell population is referred to as Forward Programmed CNS-like ECs (fpCECs). Bulk RNA sequencing was performed at N=4 independent lentivirus transductions for each condition. FIG. 2B. Principal component analysis (PCA) on Forward Programmed CNS-like ECs and CECs overexpressing GFP as a control. A second PCA was performed on samples within the dashed box in the first PCA to observe better resolution. Each dot on graph represents an independent biological replicate. FIG. 2C. Euclidean distance between the transcriptomes of fpCECs and the average transcriptome of CECs overexpressing GFP is plotted. Overexpression of KLF family (KLF2, KLF4), AP-1 family (FOS, JUN) and FOXF family (FOXF1, FOXF2) TFs induced the most significant global gene expression changes. Method for calculating Euclidean distance is documented in methods section. FIG. 2D. Heatmap of the expression of 88 BBB-enriched genes for Forward Programmed CNS-like ECs overexpressing each of the 21 candidate TFs. z-scores normalized to the mean expression for each of the 88 BBB-enriched genes are plotted. The rightmost column is GFP control. Average of N=4 biological replicates plotted. FIG. 2E. BBB score analysis of Forward Programmed CNS-like ECs overexpressing each of the 21 candidate TFs. Overexpression of KLF family (KLF2, KLF4), ZIC family (ZIC2, ZIC3) and FOX family (FOXF1, FOXF2, FOXC1, FOXQ1) TFs generates samples with the highest BBB scores. Method for calculating BBB score is documented in methods section. FIG. 2F. Heatmap of the expression of 21 candidate TFs upon overexpression of the same 21 candidate TFs individually. z-scores normalized to the mean expression for each of the 21 candidate TFs are plotted. Average of N=4 biological replicates plotted. FIG. 2G. String-style map for gene expression regulations among the 21 candidate TFs. If TF A overexpression drives the expression of TF B more than two-fold in a statistically significant manner, an arrow from TF A to TF B is plotted. A p_{adj}<0.05 in Wald test followed by Benjamini-Hochberg correction was used as the statistical significance cutoff.

FIG. 3A-3R. KLF family TFs induce significant BBB-like gene and protein expression through synergy with canonical Wnt signaling. FIG. 3A. Expression of Klf2 in mouse brain and non-brain (heart, lung, liver, kidney and skeletal muscle) ECs in single cell transcriptomic analysis. FIG. 3B. Expression of Klf4 in mouse brain and non-brain (heart, lung, liver, kidney and skeletal muscle) ECs in single cell transcriptomic analysis. FIG. 3C. Expression of KLF2 in human brain capillary and tip ECs in single cell transcriptomic analysis. FIG. 3D. Expression of KLF4 in human brain capillary and tip ECs in single cell transcriptomic analysis. FIG. 3E. Log ratio-average (MA) plot for differential gene expression between Forward Programmed CNS-like ECs overexpressing KLF2 and CECs overexpressing GFP. x-axis is the log 10 mean of normalized counts in RNASeq. y-axis is the log 2 fold change of gene expression. BBB-enriched and depleted genes are marked red. FIG. 3F. Diagram demonstrating the effect of overexpression of KLF family TFs on 88 BBB-enriched genes. FIG. 3G. Heatmap of the expression of efflux transporter, specialized transporter, junction genes and transcription factors that were upregulated by KLF2 and KLF4 overexpression. z-scores normalized to the mean expression for each of the 88 BBB-enriched genes are plotted. N=4 biological replicates per condition. Forward programmed CECs received either a control GFP lentivirus, or KLF2 lentivirus, or KLF4 lentivirus at MOI=2. FIG. 3H. Immunocytochemistry of efflux transporter (ABCB1, ABCG2), specialized transporter (SLC2A1, SLC38A5), junction (LSR, OCLN) and endocytosis-related gene (CAV1) in Forward programmed CECs receiving either an empty lentivirus vector, or KLF2 lentivirus at MOI=2. FIG. 3I. Quantification of immunocytochemistry images. N=3 biological replicates per condition. MFI: mean fluorescence intensity. *: p<0.05; **: p<0.01; ***: p<0.001 in Student's t-tests. FIG. 3J. Heatmap of the expression of target genes downstream of the ERK5 signaling pathway. N=4 biological replicates per condition. Forward programmed CECs receiving either a control GFP lentivirus, or KLF2 lentivirus, or KLF4 lentivirus at MOI=2. Color bar same as in panel g. FIG. 3K. Heatmap of the expression of a panel of BBB enriched genes with functional annotation, including efflux transporters (ABCB1, ABCG2), specialized transporters (MFSD2A, SLC2A1, SLC38A5, SLCO2B1, TFRC), junction protein (LSR), and transcription factors (FOXF2, FOXQ1, LEF1, ZIC3). Samples are CECs receiving either Wnt agonist CHIR99021 or DMSO control, KLF2 lentivirus or GFP control lentivirus. Average of N=4 biological replicates per condition are plotted. Color bar same as in panel g. FIG. 3L. Immunocytochemistry of glucose transporter SLC2Alin Forward programmed CECs receiving either Wnt agonist CHIR99021 or DMSO control, KLF2 lentivirus or empty lentivirus. FIG. 3M. Quantification of SLC2A1 immunocytochemistry images. N=4 biological replicates per condition. ***: p<0.001; ****: p<0.0001 in Two-way ANOVA followed by Tukey's t-test. FIG. 3N. Heatmap of the expression of canonical Wnt signaling TCF/LEF family TFs, and downstream genes of TCF/LEF family TFs. Samples are CECs receiving either Wnt agonist CHIR99021 or DMSO control, KLF2 lentivirus or GFP control lentivirus. Average of N=4 biological replicates per condition are plotted. Color bar same as in panel g. FIG. 3O. Quantification of Geometric Mean of eGFP expression using 7xTCF-eGFP H9 (7TGP) Wnt reporter line derived EPCs transduced with either empty vector or KLF2 lentivirus and cultured with or without CHIR99021. FIG. 3P. Quantification of LEF1, FIG. 3Q. Quantification of TCF7, and FIG. 3R. Quantification of SLC2A1 gene expression in HUVECs transduced with transduced with empty vector or KLF2 lentivirus and cultured with or without CHIR99021.

FIG. 4A-4E. KLF family TFs induce significant BBB-like gene and protein expression. FIG. 4A. Dot plot of Klf2 and Klf4 expression in mouse brain and non-brain ECs in single cell transcriptomic analysis; and dot plot of KLF2 and KLF4 expression in human brain tip and capillary ECs in single cell transcriptomic analysis. FIG. 4B. MA plot for differential gene expression between Forward Programmed CNS-like ECs overexpressing KLF4 and CECs overexpressing GFP. x-axis is the log 10 mean of normalized counts in RNASeq. y-axis is the log 2 fold change of gene expression. BBB-enriched and depleted genes are marked red. FIG. 4C. Heatmap of the expression of enzymes and signaling-related genes that are upregulated by KLF2 and KLF4 overexpression. z-scores normalized to the mean expression for each of the 88 BBB-enriched genes are plotted. N=4 biological replicates per condition. Forward programmed CECs receiving either a control GFP lentivirus, or KLF2 lentivirus, or KLF4 lentivirus at MOI=2. FIG. 4D. Immunocytochemistry of transcription factor (LEF1), specialized transporter (SLCO2B1, SLC38A5), junction (LSR, OCLN) and endocytosis-related gene (CAV1) in Forward programmed CECs receiving either an empty lentivirus vector, or KLF4 lentivirus at MOI=2. FIG. 4E. Quantification of immunocytochemistry images. N=3 biological replicates per condition. MFI: mean fluorescence intensity. *: p<0.05; ***: p<0.001; ****: p<0.0001 in Student's t-tests.

FIG. 5A-5G. KLF2 and Wnt activation synergistically induce BBB-like gene and protein expression. FIG. 5A. Heatmap of the expression of 88 BBB-enriched genes in CECs. Samples are CECs receiving either Wnt agonist CHIR99021 or DMSO control, KLF2 lentivirus or GFP control lentivirus. N=4 biological replicates per condition are plotted. FIG. 5B. Immunocytochemistry of glucose transporter SLC38A5 in Forward programmed CECs receiving either Wnt agonist CHIR99021 or DMSO control, KLF2 lentivirus or empty lentivirus. FIG. 5C. Quantification of SLC38A5 immunocytochemistry images. N=4 biological replicates per condition. ****: p<0.0001 in Two-way ANOVA followed by Tukey's t-test. FIG. 5D. Immunocytochemistry of glucose transporter SLCO2B1 in Forward programmed CECs receiving either Wnt agonist CHIR99021 or DMSO control, KLF2 lentivirus or empty lentivirus. FIG. 5E. Quantification of SLCO2B1 immunocytochemistry images. N=4 biological replicates per condition. ***: p<0.001 in Two-way ANOVA followed by Tukey's t-test. FIG. 5F. Flow cytometry histogram of eGFP expression using 7xTCF-eGFP H9 (7TGP) Wnt reporter line derived EPCs transduced with either empty vector or KLF2 lentiviruses, with or without CHIR99021 treatment. Quantification in FIG. 3 (0). FIG. 5G. Quantification of PECAM1, CDH5 and OCLN gene expression in HUVECs transduced with transduced with empty vector or KLF2 lentivirus and cultured with or without CHIR99021. Two-way ANOVA followed by Tukey's test was used for statistical analysis.

FIG. 6A-6X. FOX and ZIC family TFs induce significant BBB-like gene and protein expressions. FIG. 6A. Expression of Foxf2 in mouse brain and non-brain ECs in single cell transcriptomic analysis. FIG. 6B. Expression of Foxfl in mouse brain and non-brain ECs in single cell transcriptomic analysis. FIG. 6C. Expression of FOXF2 in human brain and non-brain ECs in single cell transcriptomic analysis. FIG. 6D. Expression of FOXF1 in human brain and non-brain ECs in single cell transcriptomic analysis. FIG. 6E. Diagram demonstrating the effect of overexpression of FOXF family TFs on 88 BBB-enriched genes. FIG. 6F. Heatmap of the expression of genes regulating BBB phenotypes, transcription factors and lipid metabolism genes that are upregulated by FOXF2 and FOXF1 overexpression. z-scores normalized to the mean expression for each of the BBB-enriched genes are plotted. N=4 biological replicates per condition. Forward programmed CECs receiving either a control GFP lentivirus, or TF lentivirus at MOI=2. FIG. 6G. Immunocytochemistry of SLC2A1 in Forward programmed CECs receiving either an empty lentivirus vector, or TF lentivirus at MOI=2. FIG. 6H. Quantification of SLC2A1 immunocytochemistry images. N=4 biological replicates per condition. MFI: mean fluorescence intensity. ***: p<0.001 in One-way ANOVA followed by Tukey's test. FIG. 6I. Expression of Foxq1 in mouse brain and non-brain ECs in single cell transcriptomic analysis. FIG. 6.J. Expression of Foxc1 in mouse brain and non-brain ECs in single cell transcriptomic analysis. FIG. 6K. Expression of FOXQ1 in human brain and non-brain ECs in single cell transcriptomic analysis. FIG. 6L. Expression of FOXC1 in human brain and non-brain ECs in single cell transcriptomic analysis. FIG. 6M. Diagram demonstrating the effect of overexpression of FOXQ1 and FOXC1 on 88 BBB-enriched genes. FIG. 6N. Heatmap of the expression of BBB phenotypes and transcription factors that are upregulated by FOXQ1 and FOXC1 overexpression. z-scores normalized to the mean expression for each of the BBB-enriched genes are plotted. N=4 biological replicates per condition. Forward programmed CECs receiving either a control GFP lentivirus, or TF lentivirus at MOI=2. FIG. 6O. Immunocytochemistry of SLC2A1 and CAV1 in Forward programmed CECs receiving either an empty lentivirus vector, or TF lentivirus at MOI=2. FIG. 6P. Quantification of immunocytochemistry images. N=4 biological replicates per condition. MFI: mean fluorescence intensity. **: p<0.01; ***: p<0.001; ****: p<0.0001 in One-way ANOVA followed by Tukey's test. FIG. 6Q. Expression of Zic2 in mouse brain and non-brain ECs in single cell transcriptomic analysis. FIG. 6R. Expression of Zic3 in mouse brain and non-brain ECs in single cell transcriptomic analysis. FIG. 6S. Expression of ZIC2 in human brain tip and capillary ECs in single cell transcriptomic analysis. FIG. 6T. Expression of ZIC3 in human brain tip and capillary ECs in single cell transcriptomic analysis. FIG. 6U. Diagram demonstrating the effect of overexpression of ZIC family TFs on 88 BBB-enriched genes. FIG. 6V. Heatmap of the expression of BBB phenotypes and transcription factors that are upregulated by ZIC family TF overexpression. z-scores normalized to the mean expression for each of the BBB-enriched genes are plotted. N=4 biological replicates per condition. Forward programmed CECs receiving either a control GFP lentivirus, or TF lentivirus at MOI=2. FIG. 6W. Immunocytochemistry of SLC2A1 and SLC38A5 in Forward programmed CECs receiving either an empty lentivirus vector, or TF lentivirus at MOI=2. FIG. 6X. Quantification of immunocytochemistry images. N=4 biological replicates per condition. MFI: mean fluorescence intensity. ***: p<0.001; ****: p<0.0001 in One-way ANOVA followed by Tukey's test.

FIG. 7A-7H. FOX and ZIC family TFs induce a subset of BBB-like gene expression changes. FIG. 7A. Dot plot of Foxf1, Foxf2, Foxq1, and Foxc1 expression in mouse brain and non-brain ECs in single cell transcriptomic analysis. FIG. 7B. dot plot of FOXF1, FOXF2, FOXC1, and FOXQ1 expression in human brain and non-brain ECs in single cell transcriptomic analysis. FIG. 7C. MA plot for differential gene expression between Forward Programmed CNS-like ECs overexpressing FOXF1 and CECs overexpressing GFP. x-axis is the log 10 mean of normalized counts in RNASeq. y-axis is the log 2 fold change of gene expression. BBB-enriched and depleted genes are marked red. FIG. 7D. MA plot for differential gene expression between Forward Programmed CNS-like ECs overexpressing FOXF2 and CECs overexpressing GFP. x-axis is the log 10 mean of normalized counts in RNASeq. y-axis is the log 2 fold change of gene expression. BBB-enriched and depleted genes are marked red. FIG. 7E. MA plot for differential gene expression between Forward Programmed CNS-like ECs overexpressing ZIC2 and CECs overexpressing GFP. x-axis is the log 10 mean of normalized counts in RNASeq. y-axis is the log 2 fold change of gene expression. BBB-enriched and depleted genes are marked red. FIG. 7F. MA plot for differential gene expression between Forward Programmed CNS-like ECs overexpressing ZIC3 and CECs overexpressing GFP. x-axis is the log 10 mean of normalized counts in RNASeq. y-axis is the log 2 fold change of gene expression. BBB-enriched and depleted genes are marked red. FIG. 7G. MA plot for differential gene expression between Forward Programmed CNS-like ECs overexpressing FOXC1 and CECs overexpressing GFP. x-axis is the log 10 mean of normalized counts in RNASeq. y-axis is the log 2 fold change of gene expression. BBB-enriched and depleted genes are marked red. FIG. 7H. MA plot for differential gene expression between Forward Programmed CNS-like ECs overexpressing FOXQ1 and CECs overexpressing GFP. x-axis is the log 10 mean of normalized counts in RNASeq. y-axis is the log 2 fold change of gene expression. BBB-enriched and depleted genes are named.

FIG. 8A-8F. Combinatorial TF overexpression drives global gene expression of forward programmed CECs similarity to gain BBB EC signatures. FIG. 8A. Schematic showing computational prediction method to generate prediction of 11 top candidate combos for experimental testing. Methods for predicting combinations are in the methods section. FIG. 8B. Heatmap of the expression of 88 BBB-enriched genes for Forward Programmed CNS-like ECs overexpressing each of the 11 TF combos. z-scores normalized to the mean expression for each of the 88 BBB-enriched genes are plotted. The rightmost column is GFP control. FIG. 8C. Heatmap of the expression of 7 TFs used in combinatorial experiments. Overexpression of TFs used in each TF combo is visualized. FIG. 8D. Principal component analysis (PCA) on Forward Programmed CNS-like ECs receiving combo TFs, single TF and CECs overexpressing GFP as a control. FIG. 8E. Euclidean distance between the transcriptomes of TF combo-forward programmed CECs and the average transcriptome of CECs overexpressing GFP is plotted. Method for calculating Euclidean distance is documented in methods section. FIG. 8F. BBB score analysis of Forward Programmed CNS-like ECs overexpressing each of the combo TFs, top single candidate TF, or GFP control. Overexpression of combo TFs generates samples with the higher BBB scores than top single TF, suggesting synergistic effects of combinatorial expression of TFs. Method for calculating BBB score is documented in methods section.

FIG. 9A-9B. Combinatorial TF overexpression reveals synergy among TFs to induce BBB-like gene expression. FIG. 9A. Heatmap of the expression of BBB-enriched TFs in FIG. 1 for Forward Programmed CNS-like ECs overexpressing either GFP or TF combos with the highest BBB scores (Combo 4, 5 and 9). A broad activation of BBB-enrich TFs is observed. BBB TFs that are enriched in at least two out of the three scRNASeq analysis, but are not overexpressed as part of the TF combos are included in this panel. FIG. 9B. Heatmap of the expression of 88 BBB-enriched genes for Forward Programmed CNS-like ECs overexpressing TF combo 4, 5 and 9, along with the single TFs the combo condition is comprised of. Apparent induction of gene programs absent in single TF conditions is observed.

FIG. 10A-10H. Combinatorial TF overexpression drives global gene and protein expression of forward programmed CECs similarity to BBB ECs. FIG. 10A. Heatmap of the expression of efflux transporter, specialized transporter, junction genes and transcription factors that are upregulated by TF combos 4, 5 and 9 overexpression. z-scores normalized to the mean expression for each of the 88 BBB-enriched genes are plotted. N=4 biological replicates per condition. Forward programmed CECs receiving either a control GFP lentivirus, or TF combo lentiviruses. FIG. 10B. Immunocytochemistry of specialized transporter (SLC2A1, SLC38A5, SLC7A5, SLCO2B1, LSR), and junction (CLDN5, OCLN) in Forward programmed CECs receiving either an empty lentivirus vector, or Combo 4, 5, or 9 lentiviruses. FIG. 10C. Quantification of immunocytochemistry images in (b). N=3 biological replicates per condition. MFI: mean fluorescence intensity. ***: p<0.001, ****: p<0.0001 in One-way ANOVA followed by Tukey's tests. FIG. 10D. Heatmap of the expression of canonical Wnt signaling TCF/LEF family TFs, and genes involved in the degradation of β-catenin. Samples are CECs receiving Combo 4, 5, 9 combo lentiviruses, single TF in combos (KLF2, KLF4, ZIC3, FOXF1, FOZF2, NR4A2, FOXQ1), or GFP control lentivirus. Average of N=4 biological replicates per condition are plotted. FIG. 10E. Immunocytochemistry of β-catenin in Forward programmed CECs receiving either an empty lentivirus vector, or Combo 4, 5, or 9 lentiviruses. FIG. 10F. Quantification of immunocytochemistry images in (e). N=3 biological replicates per condition. MFI: mean fluorescence intensity. *: p<0.05, **: p<0.001 in One-way ANOVA followed by Tukey's tests. FIG. 10G. Flow cytometry of eGFP expression using 7xTCF-eGFP H9 (7TGP) Wnt reporter line derived EPCs transduced with either empty vector or Combo 4, 5 or 9 lentiviruses with CHIR99021 treatment. FIG. 10H. Quantification of geometric mean of fluorescence in (g). ****: p<0.0001 in One-way ANOVA followed by Tukey's tests.

FIG. 11A-11B. Combinatorial TF overexpression does not interfere with endothelial gene expression. FIG. 11A. Immunocytochemistry analysis and FIG. 11B. quantification of endothelial genes CDH5 and PECAM1 in FPCECs treated with either empty vector lentiviruses or Combo 4, 5, or 9 lentiviruses. ns: p>0.05 in One-way ANOVA followed by Tukey's tests.

FIG. 12A-120. Expression level of select BBB genes in forward programmed CECs. Transcript per million (TPM) values in RNASeq data for GFP, Combo 4, Combo 5 and Combo 9 samples were quantified for indicated genes. One-way ANOVA followed by Tukey's tests were used as statistics analysis tool. FIG. 12A. MFSD2A expression. FIG. 12B. PLVAP expression. FIG. 12C. CAV1 expression. FIG. 12D. CAV2 expression. FIG. 12E. ABCB1 expression. FIG. 12F. ABCG2 expression. FIG. 12G. SLC2A1 expression. FIG. 12H. SLC7A5 expression. FIG. 12I. SLC3A2 expression. FIG. 12J. SLC38A5 expression. FIG. 12K. SLCO2B 1 expression. FIG. 12L. TFRC expression. FIG. 12M. CLDN5 expression. FIG. 12N. OCLN expression. FIG. 12O. LSR expression.

FIG. 13A-13R. Combinatorial TF overexpression generates forward programmed CECs that exhibit key BBB phenotypes. FIG. 13A. Flow cytometry analysis of endocytosed Albumin-Alexa Fluor 647 of forward programmed CECs receiving either combo 4, combo 5, or combo 9 TFs lentivirus, or an empty lentivirus control. Experiments were performed at 37° C. Flow histograms represent the 4 samples. FIG. 13B. Quantification of geometric mean of Albumin-Alexa Fluor 647 from flow cytometry analysis of forward programmed CECs receiving either combo 4, combo 5 or combo 9 TFs lentivirus, or an empty lentivirus control. Experiments were performed at 37° C. or 4° C. N=3 biological replicates for each condition. ****: p<0.0001 in Two-way ANOVA followed by Tukey's test. FIG. 13C. Flow cytometry analysis of endocytosed 10 kDa dextran-Alexa Fluor 488 of forward programmed CECs receiving either combo 4, combo 5, or combo 9 TFs lentivirus, or an empty lentivirus control. Experiments were performed at 37° C. Flow plots represent the 4 samples. FIG. 13D. Quantification of geometric mean of 10 kDa dextran-Alexa Fluor 488 from flow cytometry analysis of forward programmed CECs receiving either combo 4, combo 5 or combo 9 TFs lentivirus, or an empty lentivirus control. Experiments were performed at 37° C. or 4° C. N=3 biological replicates for each condition quantified. p<0.0001 in Two-way ANOVA followed by Tukey's test. FIG. 13E. Immunocytochemistry of CAV1 and PLVAP in forward programmed CECs receiving either an empty lentivirus vector, or TF combos 4, 5, or 9 lentiviruses at MOI=2 each. FIG. 13F. Quantification of CAV1 and PLVAP immunocytochemistry images. N=3 biological replicates per condition. MFI: mean fluorescence intensity. ***: p<0.001; ****: p<0.0001 in One-way ANOVA followed by Tukey's test. FIG. 13G. Flow cytometry analysis of ABCB1 expression of forward programmed CECs receiving either TF combo 4, combo 5, or combo 9 lentiviruses, or an empty lentivirus control. FIG. 13H. Quantification of geometric mean of ABCB1 from flow cytometry analysis of forward programmed CECs receiving either TF combo 4, combo 5 or combo 9 lentiviruses, or an empty lentivirus control. ****: p<0.0001 in One-way ANOVA followed by Tukey's test. FIG. 13I. Flow cytometry analysis of Rhodamine 123, a ligand of P-gp efflux pump. Flow histograms represent forward programmed CECs receiving either TF combo 4, combo 5, or combo 9 lentiviruses, or an empty lentivirus control. FIG. 13J. Quantification of geometric mean of Rhodamine 123 from flow cytometry analysis of forward programmed CECs receiving either TF combo 4, combo 5 or combo 9 lentiviruses, or an empty lentivirus control, in the presence or absence of the efflux pump inhibitor CsA. Mean±SD, **: p<0.01, ****: p<0.0001 in Two-way ANOVA followed by Tukey's test. FIG. 13K. Flow cytometry analysis of ABCG2 expression of forward programmed CECs receiving either TF combo 4, combo 5, or combo 9 lentiviruses, or an empty lentivirus control. FIG. 13L. Quantification of geometric mean of ABCG2 from flow cytometry analysis of forward programmed CECs receiving either TF combo 4, combo 5 or combo 9 lentiviruses, or an empty lentivirus control. **: p<0.0001 in One-way ANOVA followed by Tukey's test. FIG. 13M. Flow cytometry analysis of Hoechst 33342, a substrate of BCRP efflux pump. Flow histograms represent forward programmed CECs receiving either TF combo 4, combo 5, or combo 9 lentiviruses, or an empty lentivirus control. FIG. 13N. Quantification of geometric mean of Hoechst 33342, a substrate of BCRP efflux pump, from flow cytometry analysis of forward programmed CECs receiving either TF combo 4, combo 5 or combo 9 lentiviruses, or an empty lentivirus control, in the presence or absence of K0143. Mean±SD, **: p<0.01, ***: p<0.001, **: p<0.0001 in Two-way ANOVA followed by Tukey's test. FIG. 13O. Transendothelial electrical resistance (TEER) of forward programmed CECs receiving either TF combo 4, combo 5 or combo 9 lentiviruses, or GFP lentivirus. TEER measured 24 hours (Day 2) onwards after transduction, Mean±SD. FIG. 13P. Quantification of TEER measurement on day 6 of forward programmed CECs receiving either TF combo 4, combo 5, combo 9 lentiviruses, or GFP control lentivirus. Mean±SD, ***: p<0.001 in One-way ANOVA followed by Tukey's test. FIG. 13Q. Transendothelial electrical resistance (TEER) of HUVECs receiving either TF combo 4, combo 5 or combo 9 lentiviruses, or GFP lentivirus. TEER measured 24 hours (Day 2) onwards after transduction, Mean±SD. FIG. 13R. Quantification of TEER measurement on day 7 of forward programmed HUVECs receiving either TF combo 4, combo 5, combo 9 lentiviruses, or GFP control lentivirus. Mean±SD, **: p<0.01, ***: p<0.001 in One-way ANOVA followed by Tukey's test.

FIG. 14A-14B. Lentivirus treatment affects CECs and HUVECs TEER differently. FIG. 14A. TEER values were measured for CECs that were transduced with GFP lentivirus or untransduced. FIG. 14B. TEER values were measured for HUVECs that were transduced with GFP lentivirus or untransduced.

FIG. 15A-15D. Combinatorial TF overexpression reprograms HUVECs and hCMEC/D3 cells to gain BBB-like gene and protein expression. FIG. 15A. Forward programmed HUVECs were prepared by dosing lentiviruses delivering a single TF at MOI=2 for each condition. RNA was extracted five days after lentivirus transduction and RT-qPCR was performed on samples for indicated genes. Fold change of gene expression compared to the GFP control is plotted. One-way ANOVA followed by Tukey's test was used for statistical analysis. FIG. 15B. Immunocytochemistry of OCLN, CLDN5 and SLC2A1 in forward programmed HUVECs receiving either an empty lentivirus vector, or TF combo 4, 5 or 9 lentiviruses at MOI=2 each. MFI of N=3 biological replicates were quantified for each condition. One-way ANOVA followed by Tukey's test was used for statistical analysis. FIG. 15C. Forward programmed hCMEC/D3 cells were prepared by dosing lentiviruses delivering a single TF at MOI=2 for each condition. RNA was extracted five days after lentivirus transduction and RT-qPCR was performed on samples for indicated genes. Fold change of gene expression compared to the GFP control is plotted. One-way ANOVA followed by Tukey's test was used for statistical analysis. FIG. 15D. Immunocytochemistry of CLDN5 in forward programmed hCMEC/D3 cells receiving either an empty lentivirus vector, or TF combo 4, 5 or 9 lentiviruses at MOI=2 each. MFI of N=3 biological replicates were quantified for each condition. One-way ANOVA followed by Tukey's test was used for statistical analysis.

### DETAILED DESCRIPTION

Endothelial cells of the central nervous system (CNS) acquire specialized barrier properties in response to extrinsic signals during development. Existing human blood brain barrier (BBB) models do not fully mimic the developmental trajectory or transcriptional characteristics of CNS endothelial cells. The present invention provides methods for producing an endothelial cell capable of forming a confluent monolayer with blood-brain barrier (BBB)-like properties.

One aspect of the present invention provides a method of culturing an endothelial cell or an endothelial progenitor cell in a medium with one or more transcription factors.

In some embodiments, the endothelial cell is an endothelial progenitor cell. The term “endothelial progenitor cell,” as used herein, refers to cells that are able to differentiate into endothelial cells. In some embodiments, the endothelial progenitor cell is a CD34+CD31+ cell (i.e., cells that express the cell surface antigens CD34 and CD31). These cells may also be characterized as CD34+CD31+CD144+ endothelial progenitor cells.

Endothelial progenitor cells can be isolated from other cell types using any cell sorting method known in the art. Suitable cell sorting methods include both fluorescence activated cell sorting (FACS) methods and magnetic-activated cell sorting (MACS) methods. In the Examples, the inventors isolated endothelial progenitor cells using MACS based on CD31 surface antigen expression. Examples of alternative MACS-based strategies for isolating these cells include using CD34-FITC and anti-FITC magnetic beads with an Easy Sep Magnet and using CD31-biotin and anti-biotin magnetic beads with LS Columns on a MidiMACS Magnet. In some embodiments, the endothelial progenitor cell is a central nervous system endothelial cell or isolated from the CNS.

Other sources of endothelial cells may also be used in the present method. For example, primary endothelial cells, endothelial cell lines (e.g. HUVECs), immortalized endothelial cell lines (e.g. hCMEC/D3) and animal or human-derived endothelial progenitors (e.g. cord blood-derived endothelial progenitor cells). In some embodiments, the endothelial cell is isolated from or derived from a central nervous system source.

The term “pluripotent stem cell” (PSC) refers to a cell that has the ability to differentiate into cells of all three germ layers (i.e., ectoderm, endoderm, and mesoderm). Pluripotent stem cells include embryonic stem cells (ESCs) and induced pluripotent stem cells (iPSCs). iPSCs are stem cells that are produced by genetically reprogramming a multipotent or somatic cell back to a pluripotent state. This is accomplished through forced expression of pluripotency-associated factors (e.g., Oct3/4, Sox2, Nanog, Tbx3, and Klf4/5). In preferred embodiments, the pluripotent stem cells of the present invention are human pluripotent stem cells (hPSCs).

Differentiation is the process by which a cell transitions from one cell type into another more specialized cell type. Methods for differentiating pluripotent stem cells into endothelial progenitor cells are described in U.S. Pat. No. 9,290,741, the contents of which is incorporated by reference in its entirety. By way of example, and not limitation, pluripotent stem cells of the present disclosure may be first differentiated into endothelial progenitor cells. The endothelial progenitor cells may then be forward programmed with transcription factors to further differentiate the cells into endothelial cells with BBB (blood brain barrier) properties.

Cell culture is the process by which cells are grown in an artificial environment. Cells are typically cultured in a culture medium in a vessel (e.g., a dish, flask, plate, or tube), and other factors such as the concentration of gases (e.g., CO₂, O₂), pH, osmotic pressure, and temperature may be manipulated. A “culture medium” is a substance that provides the necessary nutrients (amino acids, carbohydrates, vitamins, minerals), growth factors, and/or hormones for a cell to grow. A culture medium may be a solid, liquid, or semi-solid substance. In the Examples, the inventors cultured endothelial progenitor cells in a medium to which a Wnt/β-catenin signaling activator was added.

One aspect of the present disclosure provides a method of forward programming an endothelial cell or endothelial progenitor cell. Forward programming is a cell engineering technique which involves converting cells, such as pluripotent stem cells into a specific cell type, such as a cell with BBB properties with the forced expression of transcription factors. In some embodiments, the cells of the present disclosure are forward programmed CNS-like endothelial cells (fpCEC). The transcription factors in fpCEC may be expressed at a different level (i.e. higher or lower) than what they would normally be expressed at or expressed at a different time or in different combinations than would occur in nature. The present disclosure demonstrates that specific transcription factors or combinations of transcription factors or one or more transcription factors and a Wnt/β-catenin signaling activator can be used to generate fpCEC with BBB properties. This method is novel and unique as previous methods by other researchers failed to achieve sufficient gene expression, function attributes of the BBB and or did not achieve high barrier quality (for example as quantified by TEER). The fpCEC of the present disclosure have unique properties of brain endothelial cells, as compared to peripheral endothelial cells

The present disclosure provides a method for producing an endothelial cell capable of forming a confluent monolayer with blood-brain barrier (BBB)-like properties. In some embodiments, the endothelial cell or endothelial progenitor cell is forward programed with transcription factors to generate an endothelial cell with BBB-like properties.

In some embodiments, the endothelial cell or endothelial progenitor cell is forward programed with transcription factors and a Wnt/β-catenin activator to generate an endothelial cell with BBB-like properties. As used herein, a “Wnt/β-catenin signaling activator” is any reagent that activates Wnt/β-catenin signaling. Examples of Wnt/β-catenin activators include Wnt ligands and Gsk3 inhibitors. Examples of Wnt ligands include Wnt3a, Wnt7a, and Wnt7b. Wnt ligands may be used at concentrations ranging from about 10 ng/ml to about 200 ng/ml. Examples of Gsk3 inhibitors include small molecules that inhibit Gsk3 phosphotransferase activity or binding interactions, RNA interference reagents that result in Gsk3 knockdown (e.g., SignalSilence® GSK3α/β siRNA), and reagents that result in overexpression of a dominant negative form of Gsk3. Dominant negative forms of Gsk3 are known in the art (see, e.g., Hagen et at. (2002), J Biol Chem, 277 (26): 23330-23335). Suitable small molecule Gsk3 inhibitors include, but are not limited to, CHIR99021 (CAS No. 252917-06-9), CHIR98014 (CAS No. 556813-39-9), BIO-acetoxime (CAS No. 667463-85-6), BIO (CAS No. 667463-62-9), LiCl, SB 216763 (CAS No. 280744-09-4), SB 415286 (CAS No. 264218-23-7), AR A014418 (CAS No. 487021-52-3), 1-Azakenpaullone (CAS No. 676596-65-9), and Bis-7-indolylmaleimide (CAS No. 133052-90-1). In the Examples, the inventors utilized the Gsk3 inhibitor CHIR99021. Thus, in preferred embodiments, the Wnt/β-catenin signaling activator is CHIR99021. CHIR99021 can be used at a concentration ranging from about 3 μM to about 15 μM. In the Examples the inventors utilized CHIR99021 at a concentration of 4 μM. Thus, in preferred embodiments, the concentration of CHIR99021 in the culture ranges from about 3 μM to about 5 μM.

In some embodiments, one or more transcription factors are added to the cells. Transcription factors are proteins involved in the process of converting, or transcribing, DNA into RNA. Transcription factors often control the rate and or timing of the process of transcription and through the control of transcription these factors play a role in protein expression in the cell. Transcription factors of the present disclosure may be introduced into an endothelial cell or endothelial progenitor cell of the present disclosure by any means know in the art. By way of example and not limitation, means of introducing a transcription factor may include chemical methods, such as liposome-mediated delivery, physical methods such as electroporation, peptide assisted genome editing, such as with gene editing systems, viral methods such the use of viral vectors or through the use of plasmids.

Transcription factors of the present invention include, Dachshund homolog 1 and 2, also known as DACH1 and DACH2, Friend leukemia integration 1 transcription factor (FLI1), Fos Proto-Oncogene, AP-1 Transcription Factor Subunit (FOS), Forkhead box C1 (FOXC1), forkhead box F1 (FOXF1), forkhead box F2 (FOXF2), Forkhead box Q1 (FOXQ1), Hairy/enhancer of split protein (Hes1), Jun Proto-Oncogene, AP-1 Transcription Factor Subunit (JUN), Krüppel-like factor-2 (KLF2), Krüppel-like factor-4 (KLF4), Lymphoid enhancer-binding factor 1 (LEF1), MDS1 And EVIL Complex Locus (MECOM), Nuclear receptor 4A1 (NR4A1), Nuclear receptor 4A2 (NR4A2), Peroxisome Proliferator Activated Receptor Delta (PPARD), T-Box Transcription Factor 3 (TBX3), TSC22 Domain Family Member 1 (TSC22D1), Zic Family Member 2 (ZIC2), and Zic Family Member 3 (ZIC3). The transcription factors may be expressed in the cells using any means available to those of skill in the art. In the examples provided herein the transcription factors were expression via a lentiviral vector capable of transducing the cells and expressing the one or more transcription factors. Other viral vectors or means of gene delivery known to those of skill in the art may be used, such as transfection, transformation, lipid vesicle mediated delivery of genes via transient means or via integration into the genome of the cells to obtain expression of the transcription factors. In one embodiment, the expression of the transcription factors may be mediated via gene editing of the native site for expressing these transcription factors or via knock-in genetic editing of polynucleotides capable of mediating increased expression of the transcription factors.

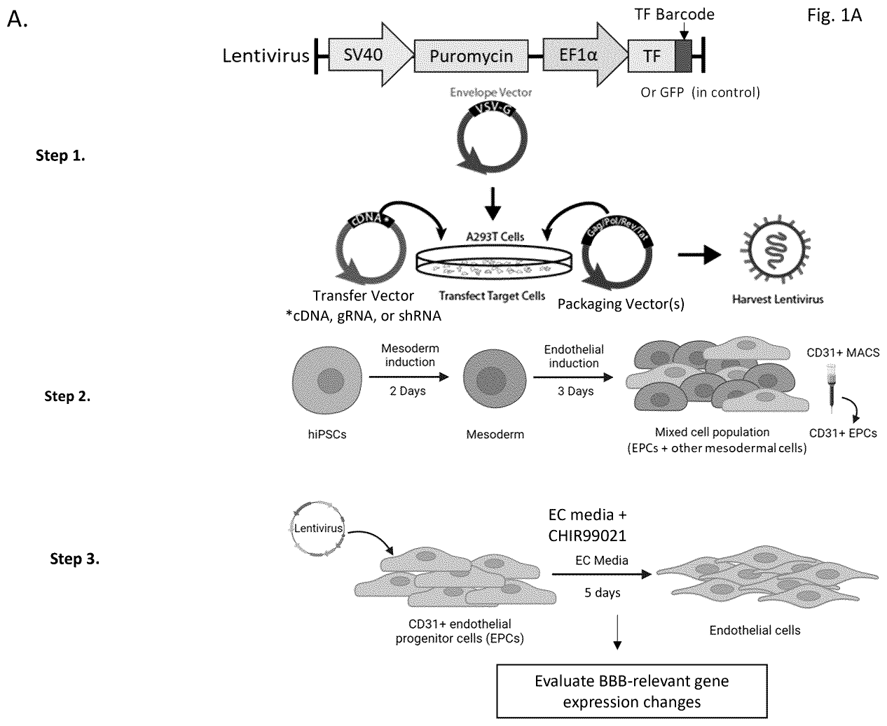

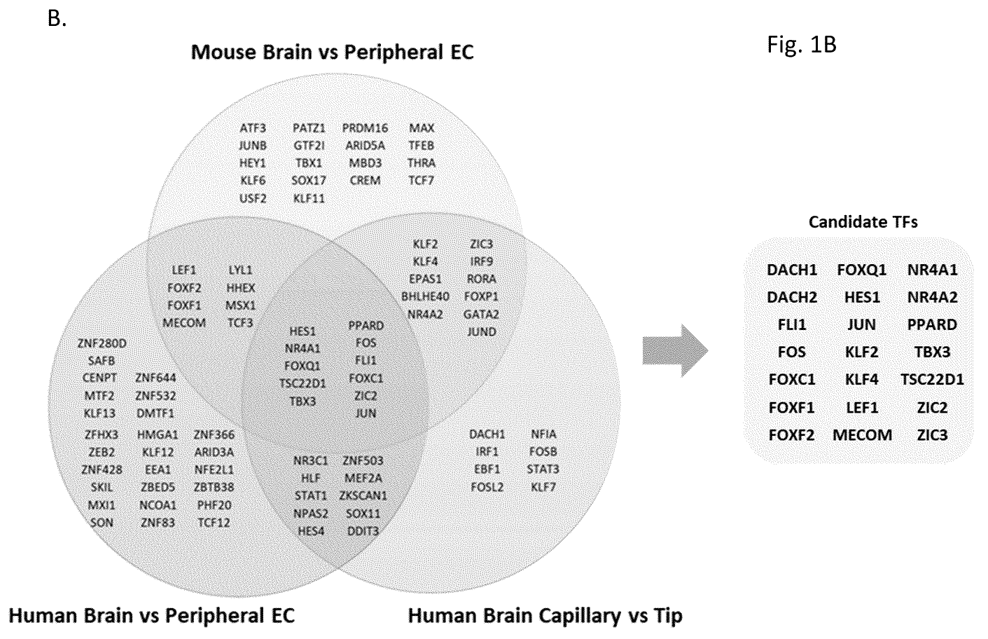

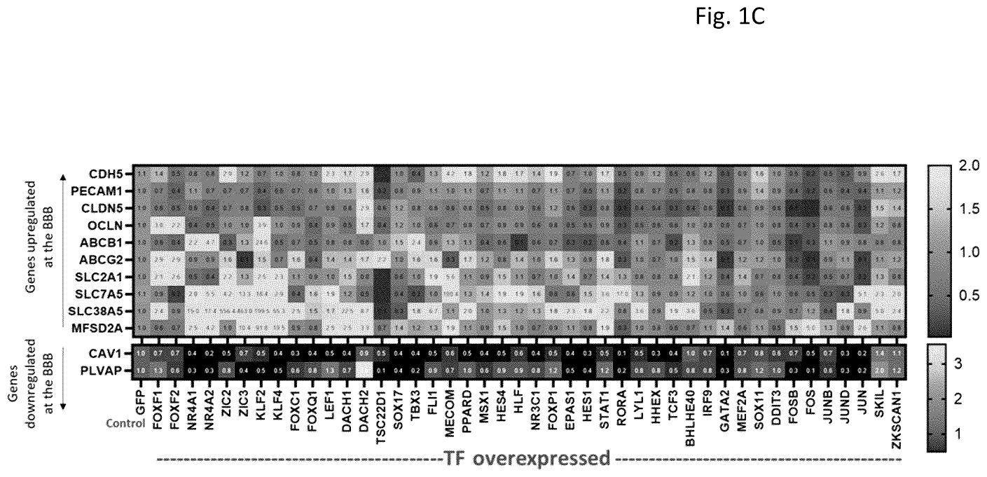

In some embodiments, one or more transcription factors may be expressed in a progenitor cell to generate a fpCEC. The transcription factors were selected to be specific for brain endothelial cells as compared to peripheral endothelial cells and specific for capillary endothelial cells with barrier function as opposed to non-barrier forming cells. The inventors initially tested 44 candidate transcription factors for their ability to induce genes enriched at the BBB. Based on these experiments, the candidate transcription factors were narrowed to a list of 21 (FIG. 1). These transcription factors include DACH1, DACH2, FLI1, FOS, FOXC1, FOXF1, FOXF2, FOXQ1, HES1, JUN, KLF2, KLF4, LEF1, MECOM, NR4A1, NR4A2, PPARD, TBX3, TSC22D1, ZIC2 and ZIC3 (FIG. 1). In some embodiments, the transcription factors of the present disclosure may comprise one or more of DACH1, DACH2, FLI1, FOS, FOXC1, FOXF1, FOXF2, FOXQ1, HES1, JUN, KLF2, KLF4, LEF1, MECOM, NR4A1, NR4A2, PPARD, TBX3, TSC22D1, ZIC2, ZIC3 and combinations thereof.

In some embodiments, the transcription factor may be a KLF (Krüppel-like factor) family transcription factor, a ZIC family transcription factor, FOX (forkhead box) family transcription factor or NR4A family transcription factor. In some embodiments, the transcription factor comprise KLF2, FOS, ZIC3, JUN, KLF4 and/or FOXF2. In some embodiments, the transcription factor may be KLF2; in some embodiments, the transcription factor may be KLF4; in some embodiments, the transcription factor may be ZIC3; in some embodiments, the transcription factor may be KLF2 and KLF4, in some embodiments, the transcription factor may be ZIC2 and ZIC3. In some embodiments, the transcription factor may be one or more of FOXF1, FOXF2, FOXQ1, and FOXC1.

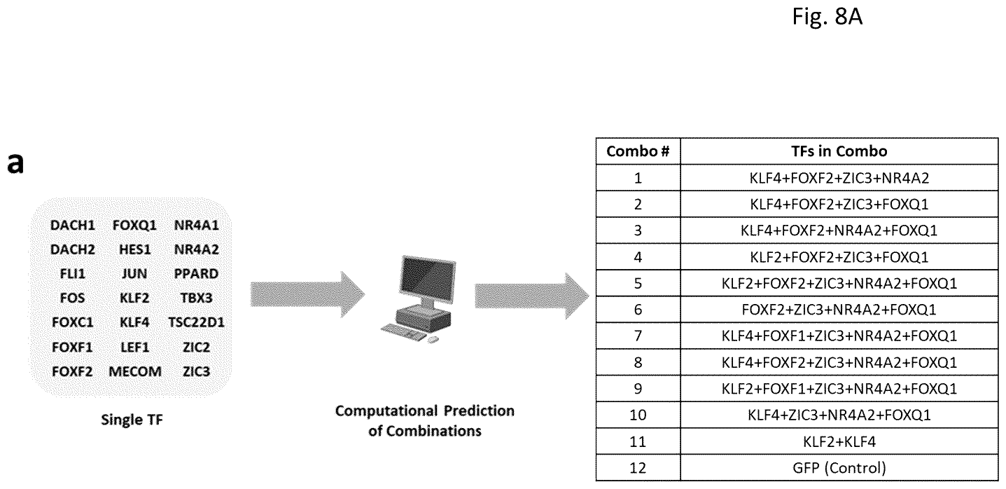

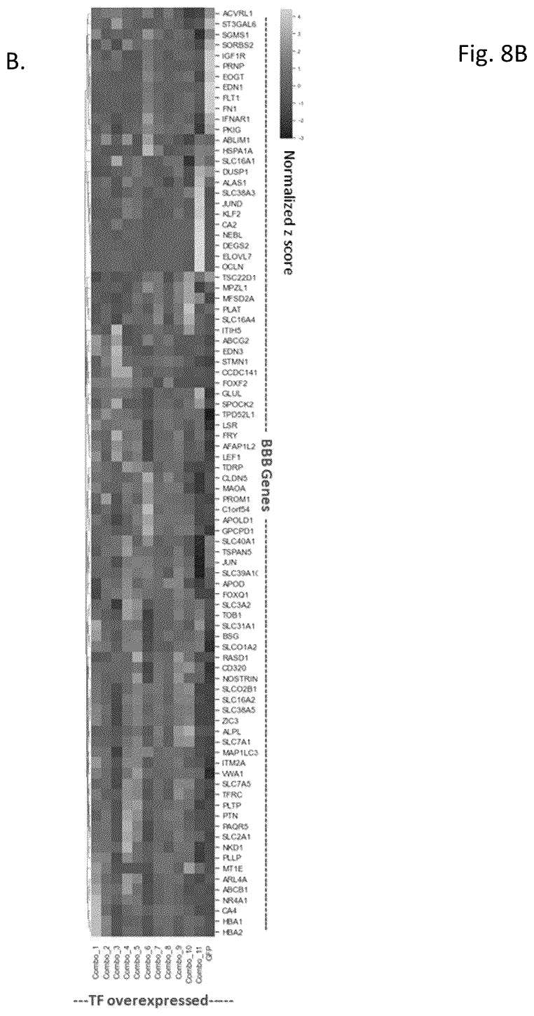

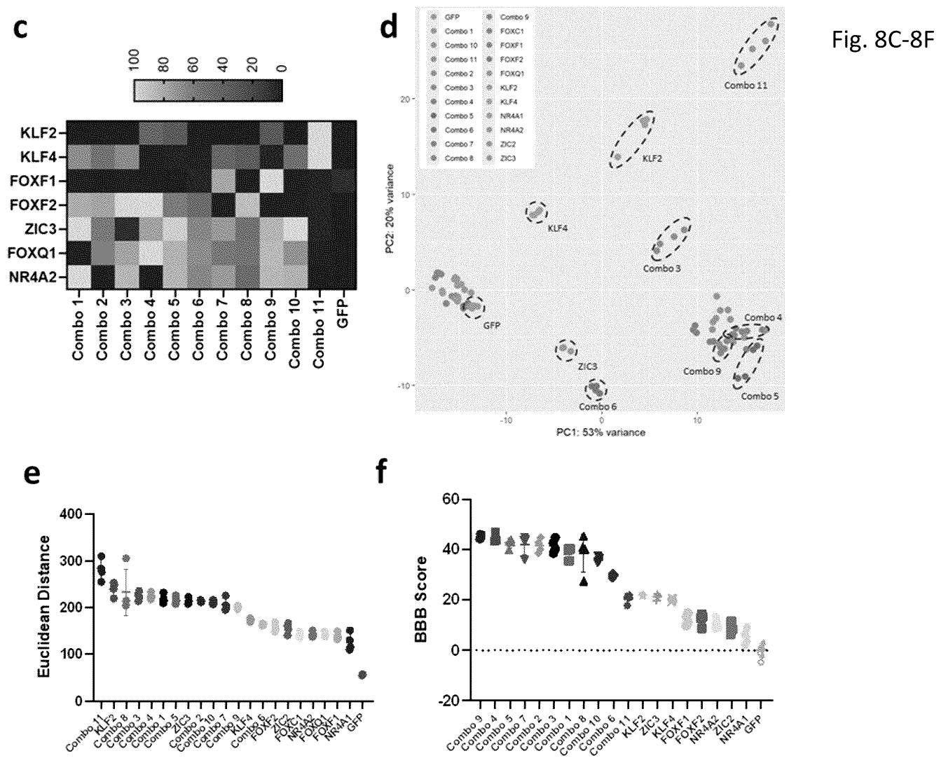

In some embodiments the one or more transcription factors may be selected from KLF2, KLF4, ZIC2, ZIC3, NR4A1, NR4A2, FOXQ1, FOXF1 and FOXF2. In some embodiments the two or more transcription factors comprise FOXF1 or FOXF2, ZIC3 or ZIC2, NR4A1 or NR4A2, FOXQ1 and KLF2 or KLF4. In some embodiments, the two or more transcription factors are selected from the groups consisting of: KLF2 or KLF4, FOXF1 or FOXF2, NR4A1 or NR4A2, and FOXQ1; or KLF2 or KLF4, FOXF1 or FOXF2, ZIC2 or ZIC3, and FOXQ1; or KLF2 or KLF4, NR4A1 or NR4A2, ZIC2 or ZIC3 and FOXQ1; or KLF2 or KLF4, FOXF1 or FOXF2, NR4A1 or NR4A2, and ZIC2 or ZIC3; or FOXF1 or FOXF2, FOXQ1, NR4A1 or NR4A2, and ZIC2 or ZIC3. In some embodiments, the two or more transcription factors comprise KLF2, FOXF1, ZIC3, NR4A2, and FOXQ1. In some embodiments the transcription factors are selected from the group consisting of KLF2, FOXF1, ZIC3, NR4A2, and FOXQ1. In some embodiments, the transcription factors are selected from KLF2, FOXF2, ZIC3, and FOXQ1. In some embodiments, the transcription factors are selected from KLF2, FOXF2, ZIC3, NR4A2 and FOXQ1. In some embodiments, the transcription factors are selected from KLF2, FOXF1, ZIC3, NR4A2 and FOXQ1. Additional combinations of transcription factors are shown in FIG. 8.

In some embodiments, the transcription factors consist of or are selected from the group consisting of DACH1, DACH2, FLI1, FOS, FOXC1, FOXF1, FOXF2, FOXQ1, HES1, JUN, KLF2, KLF4, LEF1, MECOM, NR4A1, NR4A2, PPARD, TBX3, TSC22D1, ZIC2 and ZIC3.

In some embodiments, the endothelial progenitor cells and Wnt/β-catenin signaling activator and lentiviruses or other means for expression or overexpression of transcription factors are cultured together for 2 days, 3 days, 4 days, 5 days, 6 days, 7 days or more as show in U.S. Patent Publication No. US2023/0375530A1 which is incorporated herein in its entirety. In the Examples, the inventors culture the cells for 5 days.

In some embodiments, the transcription factors are overexpressed. Overexpression increases the amount and or duration a gene is expressed. In some embodiments, overexpression of the one or more transcription factors is achieved via transduction with a virus comprising one or more transcription factors. For example, a lentivirus which expresses one or more transcription factors may be transduced into an endothelial progenitor cell such that the endothelial progenitor cell overexpress the transduced transcription factors. Other viral strategies for transcription factor overexpression in cells include using retrovirus, adenovirus, adeno-associated virus, hybrid adenoviral vectors, herpes simplex virus, pox virus, Epstein-bar virus. Other non-viral strategies for transcription factor overexpression in cells include using lipid nanoparticles, cationic lipids, cationic polymers, lipid-polymers transfections or electroporation, ultrasound, hydrodynamics to deliver DNA, mRNA, modified RNA, CRISPR activation and other activation strategies for gene expression.

The methods of the present invention produce endothelial cells capable of forming a confluent monolayer with BBB-like properties. A confluent monolayer with BBB-like properties comprises endothelial cells of the present disclosure grown in culture such that together the cells have functional properties of a BBB. BBB functional properties include reduced vesicular trafficking, efflux transporter activity and passive barrier formation. BBB-like properties also comprise, increased tight junction protein expression, increased transporter protein expression, reduced transcytosis-related protein expression, reduced leukocyte adhesion molecule expression and combinations thereof as compared to endothelial progenitor cells not treated with the transcription factors described herein. BBB-like properties is used herein to describe endothelial cells that express BBB proteins at levels similar to in vivo BBB endothelial cells. A list of BBB related gene is found in Table 3. For example, the methods described herein may be used to produce endothelial cells with increased expression of one or more proteins comprising Occludin (OCLN), Claudin-5 (CLDN5), Major facilitator superfamily domain-containing protein 2A (MFSD2A), Solute Carrier Family 2 Member 1 (SLC2A1), solute carrier family 38 member 5 (SLC38A5), solute carrier family 7 member 5 (SLC7A5), ATP Binding Cassette Subfamily B Member 1 (ABCB1), ATP-binding cassette super-family G member 2 (ABCG2) and decreased expression of one or more proteins comprising Caveolin-1 (CAV1), Caveolin-2 (CAV2), Intercellular Adhesion Molecule 1 (ICAM1), Plasmalemma Vesicle Associated protein (PLVAP), and combination thereof. For example, tight junction properties may be measured by OCLN and or CLDN5 expression or by elevated trans-endothelial electrical resistance across cell monolayer, transporter protein properties may be measured by MFSD2A, SLC2A1, SLC38A5, SLC7A5, ABCB1, and or ABCG2 expression. Efflux activity can be measured by uptake of substrates. Transcytosis-related properties may be measured with CAV1, and or CAV2, and or PLVAP expression, and by uptake of 10 kDa dextran and albumin. Leukocyte adhesion may be measured with adhesion molecule (e.g. ICAM1) expression following cytokine (e.g. TNFα) activation.

Another aspect of the present invention provides a population of endothelial cells with BBB-like properties produced by the methods described herein. BBB-like properties include but are not limited to increased tight junction protein expression, increased transporter protein expression, reduced transcytosis-related protein expression, reduced leukocyte adhesion molecule expression as compared to untreated endothelial cells or endothelial cell precursors (untreated refers to not expressing or overexpressing the transcription factors as described here.

Another aspect of the present invention provides an in vitro BBB model. In some embodiments, the in vitro BBB model comprises a confluent monolayer of the endothelial cells cultured on a surface, wherein the in vitro BBB model has BBB-like properties. BBB-like properties include but are not limited to increased tight junction protein expression, increased transporter protein expression, reduced transcytosis-related protein expression, reduced leukocyte adhesion molecule expression as compared to untreated endothelial cells.

The BBB models comprise a confluent monolayer of cells. “Confluency” describes the degree to which the surface of a cell culture vessel is covered by adherent cells. Cultured cells are considered “confluent” if the surface is completely covered with cells, and there is no more room for the cells to grow as a monolayer. The term “monolayer” refers to a single layer of cells. Cells in a monolayer grow side-by-side on the same growth surface rather than on top of one another.

Suitable cell growth surfaces are known in the art and include, but are not limited to, permeable supports, collagen coated permeable membranes, filters, polymers, meshes, matrices, membranes, and hydrogel-based substrates. The surface may be within a tissue culture system or a microfluidic device. Examples of suitable permeable supports include tissue culture plate inserts, porous and permeable membranes, and transwell systems (e.g., Corning Transwells®).

In some embodiments, the BBB model is an isogenic model. As used herein, the term “isogenic model” refers to a model made from cells that are selected to model the genetics of a specific subject in vitro. An isogenic BBB model is made by generating induced pluripotent stem cells (iPSCs) from the subject's somatic cells (i.e., by reprogramming them to a pluripotent state), differentiating the iPSCs into endothelial progenitor cells using methods previous described and available to those of skill in the art and then using the methods described herein to differentiate the endothelial progenitor cells into endothelial cells with BBB-like properties, and culturing the resulting cells on a suitable surface to generate a BBB model.

BBB models can be used for a variety of purposes. For example, BBB models can be used to predict permeability of therapeutics across the BBB, study whether drug candidates would disrupt BBB properties, study biological pathways regulating the formation and maintenance of the BBB, model the BBB aspect of Alzheimer's Disease, Parkinson's Disease and other neurological diseases.

The “subject” may be a mammal or a non-mammalian vertebrate, such as a bird. Suitable mammals include, but are not limited to, humans, cows, horses, sheep, pigs, goats, rabbits, dogs, cats, bats, mice, and rats. In certain embodiments, the subject is a lab animal (e.g., a mouse or rat) and the BBB model is used for research purposes. In preferred embodiments, the subject is a human.

In some embodiments, the BBB model is an isogenic model generated from the cells of a subject that has a brain disease. In these embodiments, the BBB model can be used to screen for therapeutics that are able to cross the subject's BBB to treat the brain disease. Use of autologous cells may provide insight to a particular subject's disease or BBB. Allogeneic cells may also be used. In one alternative, the cells may comprise a genetic mutation associated with a disease or with a central nervous system or brain disorder. The genetic mutation may be identified genetically or could be unknown or as yet not identified. Examples of brain diseases include, without limitation, Alzheimer's disease, multiple sclerosis, stroke, epilepsy, traumatic brain injury, Parkinson's disease, and brain tumors. In some embodiments, the stem cells, progenitor cells or endothelial cells of the present disclosure are engineered to incorporate genetic mutation associated with disease. For example, a fpCEC may also comprise a mutation associated with Alzheimer's disease, multiple sclerosis, stroke, epilepsy, Parkinson's disease, or brain tumors.

In another aspect, the present invention provides methods for using the BBB models described herein. The BBB models can be used as a research tool or for pre-clinical studies of trans-BBB transport of therapeutic agents.

A “therapeutic agent” is an agent that aids in the treatment, prevention, or diagnosis of a disease or condition. Examples of therapeutic agents include pharmaceuticals, biologics, toxins, alkylating agents, enzymes, antibiotics, antimetabolites, antiproliferative agents, chemotherapeutic agents, hormones, neurotransmitters, oligonucleotides, aptamers, lectins, compounds that alter cell membrane permeability, photochemical compounds, small molecules, liposomes, micelles, gene therapy vectors, viral vectors and vaccines.

In some embodiments, the therapeutic agent is an agent that needs to cross the BBB to reach its target for therapy or to have a therapeutic effect. The ability of a therapeutic agent to cross the BBB can be assessed using a transwell assay. In this assay, the BBB model comprises a confluent monolayer of endothelial cells cultured on a porous membrane that separates two chambers, and the transport of substances between the two chambers is measured. The BBB model provided herein will more accurately reflect the ability of therapeutic agents to cross the BBB in vivo.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Unless otherwise specified or indicated by context, the terms “a”, “an”, and “the” mean “one or more.” For example, “a molecule” should be interpreted to mean “one or more molecules.” As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus≤10% of the particular term and “substantially” and “significantly” will mean plus or minus >10% of the particular term.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

The following examples are meant only to be illustrative and are not meant as limitations on the scope of the invention or of the appended claims.

### EXAMPLES

The following Examples are illustrative and should not be interpreted to limit the scope of the claimed subject matter.

### Example 1

Forward Programming Identifies Inducers of Blood-Brain Barrier Properties in hPSC-Derived Endothelial Cells

The blood-brain barrier (BBB) consists of highly selective endothelial cells that separate the bloodstream from the central nervous system. In vitro models of the BBB that closely resemble the in vivo physiology enable developmental studies of the BBB, neurovascular disease modeling, and drug screening. The mechanisms driving the acquisition of many BBB properties that are characteristic of brain microvascular endothelial cells (BMECs) such as tight junction formation, efflux transporter activity, and low transcytosis are still unknown. To this end, we identified TFs that are enriched in brain ECs using publicly available single cell RNA-sequencing datasets for ECs from brain and various peripheral organs in human and mice. We screened 46 of the brain EC-enriched TFs by lentivirus-based overexpression in hPSC-derived naïve ECs and evaluated the expression of BMEC-relevant genes using RT-qPCR. Bulk RNA-sequencing of the top 22 TFs revealed induction of a subset of genes related to tight junction formation (CLDN5 and OCLN), efflux and solute transport (ABCB1, ABCG2, SLC2A1), and low transcytosis (PLVAP and CAV1). Furthermore, combinatorial TF overexpression resulted in a much more substantial shift of BMEC-relevant genes compared to individual TFs. As a few examples, we observed an increase in P-gp and BCRP efflux transporter gene and protein expression. These molecular changes translated to increased efflux activity measured using P-gp and BCRP specific substrate and inhibitor combinations. We also observed a reduced expression in PLVAP and CAV1, at the gene and protein expression level, which further correlated with reduced uptake of fluorescent Dextran and Albumin, indicating overall reduced endocytosis. We also observed modest changes in barrier integrity. Interestingly, the overexpression of the same combination of TFs in HUVECs also induced a wide array of BMEC-relevant gene expression changes. Our study underscores the identification of pivotal transcription factors that play a role in inducing BBB properties, providing foundational insights in BBB development and physiology that could ultimately lead to improved in vitro BBB models.

### Methods:

Our method uses the overexpression of one or a combination of transcription factors in endothelial or endothelial progenitor cells to induce blood-brain barrier gene expression and phenotype. Human pluripotent stem cells (hPSCs) are differentiated into endothelial cells (ECs). During the differentiation, cell populations are treated with small molecule CHIR99021 along with overexpression of one or a combination of key blood brain-barrier transcription factors. Overexpression of one or a combination of these transcription factors induces global transcriptomic similarity to in vivo blood-brain barrier endothelial cells, including changes that reflect key blood-brain barrier properties. In addition to hPSC-derived endothelial cells, similar treatment of primary endothelial cells and endothelial cell lines also induce BBB-like gene expression. These include increased tight junction protein expression (OCLN and CLDN5), increased transporter protein expression (MFSD2A, SLC2A1, SLC38A5, SLC7A5, ABCB1, and ABCG2) and reduced transcytosis-related protein expression (CAV1 and CAV2) and reduced leukocyte adhesion molecule expression (ICAM1). Resultant cells also gain functions that reflect key BBB properties. These functions include higher barrier resistance (e.g. trans-endothelial electrical resistance), enhanced efflux activity (e.g. efflux of ABCB1 and ABCG2 substrates) and reduced endocytosis (e.g. reduced endocytosis of albumin and 10 kDa dextran).

### Example 2

Combinatorial Forward Programing Identifies Transcription Factors that Induce Blood-Brain Barrier Properties in Human Pluripotent Stem Cell-Derived Endothelial Cells

The blood-brain barrier (BBB) is a highly specialized interface essential for maintaining brain homeostasis and protecting the central nervous system from blood-borne substances. Brain microvascular endothelial cells (BMECs) comprising the BBB, are characterized by tight junctions, efflux transporters and low levels of transcytosis, among other specialized attributes. Despite the growing body of molecular level data that can be used to characterize BBB attributes, our understanding of the drivers of BBB property induction during BMEC development remains limited. The inventors mined single-cell RNA sequencing datasets from brain and peripheral endothelial cells to identify TFs that could be critical for orchestrating BBB development and maintenance. These forty-four TFs were overexpressed in naïve human pluripotent stem cell-derived endothelial cells to identify TFs capable of directing acquisition of BBB lineage-specific properties via a process known as forward programming. A subset of TFs including KLF2, KLF4, FOXF1, FOXF2, ZIC2, ZIC3, NR4A1, NR4A2, FOXC1, and FOXQ1 induced distinct subsets of BBB-like gene expression profiles. Moreover, the inventors identified TF combinations capable of synergistically inducing a more comprehensive BBB transcriptome. Specifically, in hPSC-derived ECs, combinations of KLF2 or KLF4 with FOXF1 or FOXF2, ZIC3, NR4A2, and FOXQ1 enhanced the expression of 62 out of 88 canonical BBB markers, yielding ECs with improved barrier function, reduced endocytosis, and increased efflux activity. The resultant forward programmed CNS-like ECs (fpCECs) offer promising new tools for modeling human BBB development, neurovascular disease and drug screening.

### INTRODUCTION

Transcription factors (TFs) are a class of proteins with DNA binding capabilities, which serve as the master regulators of gene expression. TFs play pivotal roles in controlling gene expression, influencing developmental trajectory, promoting cell lineage-specific characteristics, and thereby establishing the unique identity of cells (Lambert et al. 2018). TF overexpression can be used to transdifferentiate cells across different lineages (van der Meulen and Huising 2015; H. Wang et al. 2015), reprogram somatic cells into a pluripotent state (Takahashi and Yamanaka 2016), and the differentiation of stem cells into different lineages, including hepatocytes, pancreatic β cells, neurons, astrocytes and microglia. (Watanabe et al. 2014; Csatári, Wiendl, and Pawlowski 2024; Gu, Cromer, and Sumer 2021; Tomaz et al. 2022; Pawlowski et al. 2017). The process of using TFs to pattern cell fate in stem cell differentiations is commonly known as forward programing. The application of direct TF overexpression in driving cell fate changes is not surprising, as it recapitulates aspects of cellular differentiation driven by signaling pathways. Namely, when cells were exposed to different external stimuli, such as signaling ligands, growth factors, oxygen level changes, temperature changes, cell-cell contact and shear stress, different signaling pathways would be activated. Activation of signaling pathways leads to direct downstream gene expression changes of TFs. For example, activation of canonical Wnt signaling increases the expression of some TCF/LEF family TFs (Cadigan and Waterman 2012); activation of Notch signaling increases the expression of some HES-related family basic Helix-Loop-Helix TFs (Bray 2006). Direct expression of TFs bypasses the need for external cues, which presents especial value because many signaling pathways are hard or inaccurate to modulate in vitro. For example, activation of Notch signaling is challenging since it requires direct contact between ligand-presenting cells and receptor-bearing cells for initiation of signaling (Bray 2006). Hypoxia signaling requires either special culture environment with low oxygen, or the less physiologically mimicking use of chemicals such as cobalt chloride (Pavlacky and Polak 2020). Activation of shear stress-related signaling in vitro commonly employ microfluidic devices to replace static cell culture. But these methods face challenges of complexity in design and fabrication, low scalability and limited strength of shear induction (Simitian et al. 2022). Sometimes, for well-studied signaling pathways with known signaling ligands, differences between cells developing in vitro and in vivo could lead to the missing of cell surface receptors for these ligands, rendering the ligand treatment ineffective in vitro. Thus, forward programing presents a feasible way for recapitulating and assessing the effect of signaling pathways that are hard to access in vitro. With its benefits, forward programing also comes with its own challenges. Common problems include inaccuracy in temporal and dosage control of TF overexpression, hardship in recapitulating synergistic effects of combinatorial TF overexpression, inefficiency of gene delivery methods, and silencing of gene constructs (Bestor 2000; Sharma et al. 2021; Martinez-Corral et al. 2023).

Recently, engineering approaches using forward programing to differentiate endothelial cells from hPSCs have been developed and optimized. The TF ETV2 has been shown to directly convert hPSCs into ECs (Ng et al. 2020; K. Wang et al. 2020). SOX17, along with the supplementation of growth factor FGF2, has also been shown to drive similar conversions (Ream et al. 2024). Brain endothelial cells are specialized endothelial subtypes, with the acquisition of a unique sets of properties, including formation of tight junctions, reduced vesicular endocytosis, and the expression of specialized transporters. Previous efforts have also been attempted to convert non-barrier ECs to brain-like ECs using TFs. The overexpression of FOXF2, FOXQ1, and ZIC3 individually in human umbilical vein endothelial cells (HUVECs), increased the expression of some BMEC-relevant genes, however, the fold changes observed were very small (Hupe et al. 2017). SOX18, SOX7, ETS1, and LEF1, when overexpressed together in hPSC-derived ECs, have been reported to induce minor improvements in tight junctions as measured by TEER (Roudnicky et al. 2020). Although these changes induced by TF overexpression are trending in the correct direction, insufficient gene expression and functional attributes limit the applications of these models. Some of the potential reasons for the undesired changes could include not identifying key TFs, lack of the combination of TFs, limited cell fate plasticity in terminally differentiated cells, and epigenetic inaccessibility to TF-binding sites due to closed chromatin in specific cell type for engineering. Overall, efforts are still needed to identify the TFs responsible for the critical phenotypes of the ECs at the BBB.

A recent plethora of single cell or single nucleus transcriptomics datasets on brain endothelial cells provided rich transcriptomics datasets to help identify differentially expressed genes between brain barrier ECs and other EC subtypes. Some notable publications include mouse single cell transcriptome atlas of ECs in different organs (Kalucka et al. 2020), single cell transcriptome of the human brain vasculature (Garcia et al. 2022; Winkler et al. 2022), human single cell transcriptome atlas of brain vascular cells in Alzheimer's Disease (Yang et al. 2022; Sun et al. 2023), human single cell vasculature in prenatal brain during development (Crouch et al. 2022), human single cell atlas for neuroretina vascularization and blood-retina-barrier formation (Zarkada et al. 2021). The rich in vivo sequencing data allows us to probe brain ECs during different developmental stages, in different regions, and in health or disease. In this disclosure, we identified enriched TFs at the BBB using these single cell transcriptomics datasets.

The source of ECs is also of particular interest. Since we are trying to recapitulate barrier-genesis of ECs in vitro, plasticity of ECs in response to TF overexpression is important. Generally, it is considered that terminally differentiated cells, compared to progenitor cells and stem cells, lose fate plasticity due to a less accessible chromatin by TFs (Mansisidor and Risca 2022). Thus, we chose to forward program hPSC-derived endothelial progenitor cells (EPCs). hPSC-derived endothelial models are of high scalability (Ding, Shusta, and Palecek 2021), and have higher fate plasticity during differentiation compared to terminally differentiated cell types, such as HUVECs (Gastfriend, Nishihara, et al. 2021). In this work, we first differentiated hPSCs to EPCs, and then overexpress TFs in EPCs during further differentiation to ECs. Our approach leverages a relatively plastic progenitor state, while ensures the endothelial fate by first patterning the fates towards endothelial progenitor. Overall, our work utilized forward programing techniques to drive methodological advancements to achieve brain-like EC population from hPSCs. The new model offers gene expression similarity to in vivo brain ECs and captures key BBB phenotypes.

### Methods and Materials

Maintenance of hPSCs

Human pluripotent stem cells (IMR90-4 from WiCell WISCi004-B) were maintained on Matrigel (Corning 356234) coated plates in mTeSR (Stemcell Technologies 85850). hPSCs were passaged using Versene (Gibco 15040066) at 1 to 6 ratio once reaching 70% confluency. Regular mycoplasma and G-band karyotyping tests were performed.

### Differentiation of Forward-Programmed CECs

Differentiation was performed according to our previously published methods (Bao et al., 2015; Lian et al., 2014). Briefly, on day-3, singularized IMR90-4 hPSCs were seeded onto 12-well plates (Corning 3513) coated with Matrigel at a density of 30,000 cells per well in mTeSR. On day-2 and -1, medium changes with mTeSR were performed. On day 0 and day 1, cells were treated with 6 μM CHIR99021 (Tocris 4423) for 48 hours in LaSR medium. From day 2 to day 5, cells were treated with LaSR medium with 50 ng/mL VEGF (Peprotech 100-20). On day 5, typically 15% to 30% of cell populations are CD31+. On day 5, EPCs were purified by magnetic sorting of CD31+ cells, using anti-CD31-biotin antibody (Miltenyi 130-110-667), anti-biotin microbeads (Miltenyi 130-097-046) and a QuadroMACS separator (Miltenyi 130-091-051). Magnetic sorting was performed according to manufacturer's recommendations.

Purified CD31+ EPCs were further differentiated to CECs according to our previously published method (Gastfriend, Nishihara, et al. 2021). Briefly, Collagen IV (Sigma-Aldrich C5533) was used to coat tissue-culture plates at 10 ug/cm². hECSR media was prepared by mixing hESFM (Gibco 11111044) with 2% B-27 (Gibco 17504044) and 20 ng/mL FGF2 (Peprotech 100-18B). EPCs obtained as described above were suspended in hECSR medium supplemented with 4 Um CHIR99021 (Tocris 4423) and plated at approximately 3×10⁴ cells/cm². Lentiviruses delivering TFs were dosed along with cell plating at MOI=2 for each lentivirus. Media was changed every 48 hours. After 5 days in culture, fpCECs were analyzed.

### TF Plasmids and Lentivirus Production

TF lentivirus transfer plasmids were obtained from Addgene (Addgene 137000) as part of the MORF library lentivirus collection (Joung et al. 2024) as gifts from Feng Zhang. The lentiviral packaging plasmids psPAX2 (Addgene 12260) and pMD2.G (Addgene 12259) were obtained from Addgene as gifts from Didier Trono. To produce lentivirus, Lenti-X 293T cells (Takara 632180) were maintained on collagen I-coated (Gibco A1048301) six-well plates in DMEM (Life Technologies 11965092) supplemented with 10% FBS (Peak Serum), 1 mM sodium pyruvate (Life Technologies 11360070), and 0.5× GlutaMAX Supplement (Life Technologies 35050061). When 293T cells reached 90% confluence, psPAX2 (1 μg per well), pMD2.G (0.5 μg per well), and lentivirus transfer plasmids (1.5 μg per well) were co-transfected using FuGENE HD Transfection Reagent (9 μl per well) (Promega E2311). Medium was replaced 16 hours after transfection, and virus-containing supernatants were collected 24, 48, and 72 hours later. Supernatants were filtered through a 0.45-μm filter and concentrated 100× using Lenti-X Concentrator (Takara 631231). Titer of lentivirus was determined using Lenti-X GoStix Plus (Takara 631280) according to manufacturer's recommendations.

### Single Cell RNASeq Analysis

We obtained single cell RNA-seq gene expression matrices from GEO or ArrayExpress (GSE134355, GSE76381, GSE119212, GSE106118, GSE124395, GSE130646, GSE175895, E-MTAB-8077). We used R (version 3.6.2) and the Seurat package (Hao et al. 2024, 2021; Stuart et al. 2019; Butler et al. 2018; Satija et al. 2015) for all analyses. Data was normalized to 2000 highly variable features (genes) for each dataset independently. We performed principal component analysis and used the first 30-50 principal components to perform UMAP embedding. For marker identification, we used the FindAllMarkers function, and selected genes with adjusted P-values<0.05 based on the default Wilcoxon rank sum test (a non-parametric test that does not assume normally-distributed data) with Bonferroni correction. We used the Seurat functions DimPlot, FeaturePlot, VlnPlot, DoHeatmap, and DotPlot for visualization. To integrate different human brain scRNA-seq datasets, we used methods previously developed (Gastfriend, Foreman, et al. 2021). We used FindMarkers function from Seurat to perform differential expression tests. Statistical analyses of differential expression used the Wilcoxon rank sum test with Bonferroni correction.

### Bulk RNASeg Library Preparation and Bioinformatics Analysis

RNA-seq was performed on fpCECs from the IMR90-4 hPSC line. Four independent biological replicates were performed as described in previous section. Cells were resuspended in Tri Reagent (Zymo R2050-1-200) for cell lysis. RNA was then extracted using Direct-zol RNA Miniprep kit (Zymo R2050) according to manufacturer's guidelines. RNA quality was assayed using Agilent 2100 Bioanalyzer with Agilent RNA 6000 Pico Kit (Agilent 5067-1513). All samples have RIN number larger than 9. Poly A-enriched mRNA from 1000 ng of total RNA was then prepped using the NEBNext Poly (A) mRNA Magnetic Isolation Module (NEB E7490L) on a DynaMag-96 Side Magnet (Invitrogen 12331D). 1st strand cDNA synthesis and sequencing library preparation was performed using xGen RNA Library Preparation Kit (IDT 10009814), with library purification steps done with AMPure XP beads (Beckman Coulter A63882). Quality of the sequencing library was checked on an Agilent 2100 bioanalyzer using High Sensitivity DNA Kit (Agilent 5067-4626). DNA concentration of sequencing library was quantified on Qubit 3 with dsDNA high sensitivity kit (Invitrogen Q32851). Sequencing was performed on a NovaSeq X (Illumina), with approximately 30 million 150 bp paired end reads obtained for each sample.

FASTQ files were first tail-trimmed using Trimmomatic (Bolger, Lohse, and Usadel 2014). The trimmed reads were then aligned to the human genome (hg38) using RNA STAR (Dobin et al. 2013). Gene counts were summarized using featureCounts (Liao, Smyth, and Shi 2014). Gene counts were fed into DESeq2 (Love et al., 2014) implemented in R for differential expression analysis. The Wald test with Benjamini-Hochberg correction was used to generate adjusted p-values. Principal component analysis was performed on counts after the DESeq2 variance stabilizing transformation. Heatmap was prepared on TPM values using seaborn (Waskom 2021) in Python.

### Computational Processing to Transcriptomics Data

To quantify the level of changes induced by overexpression of each TF, we calculate the Euclidean distance between the TF-overexpression transcriptome profile to the average of GFP transcriptome profile. Euclidean distance is calculated as the sum of root squares of differences of the natural log of TPM values for each gene.

### Eucliden Distance Between Samples A and B

$$= {\sum }_{i} {( log ( {TPM}_{Gene i}^{sample A} ) - log ( {TPM}_{Gene i}^{sample B} ) )}^{2}$$

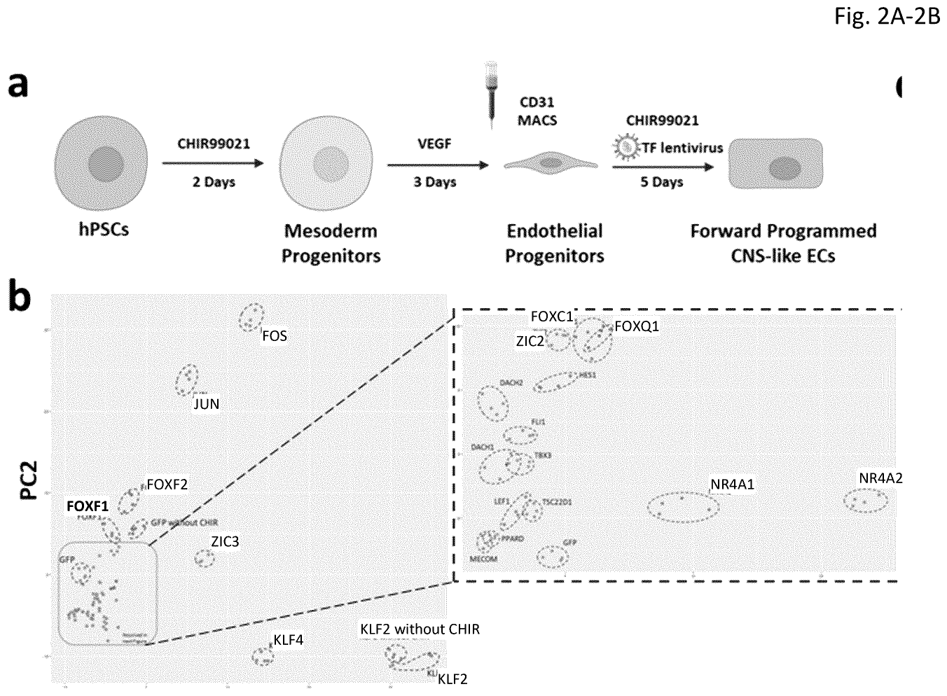

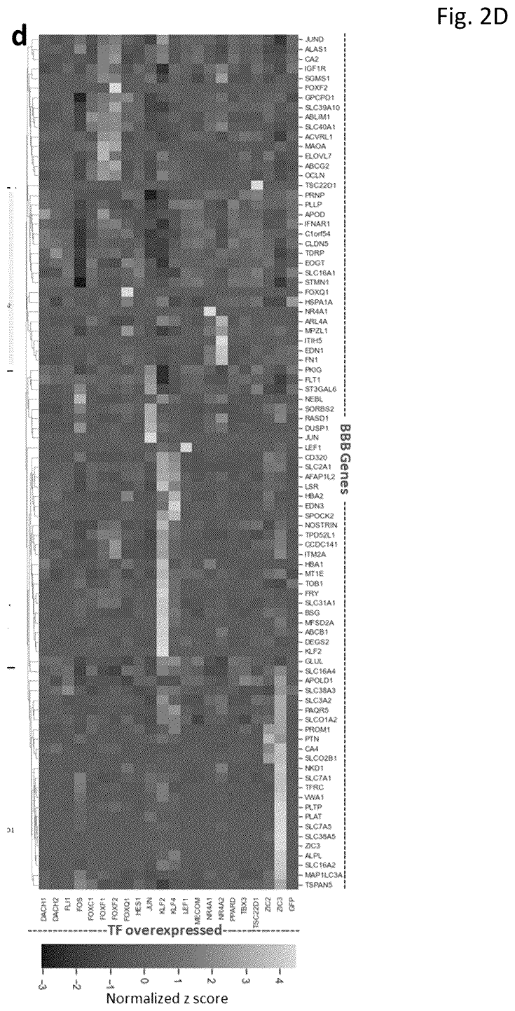

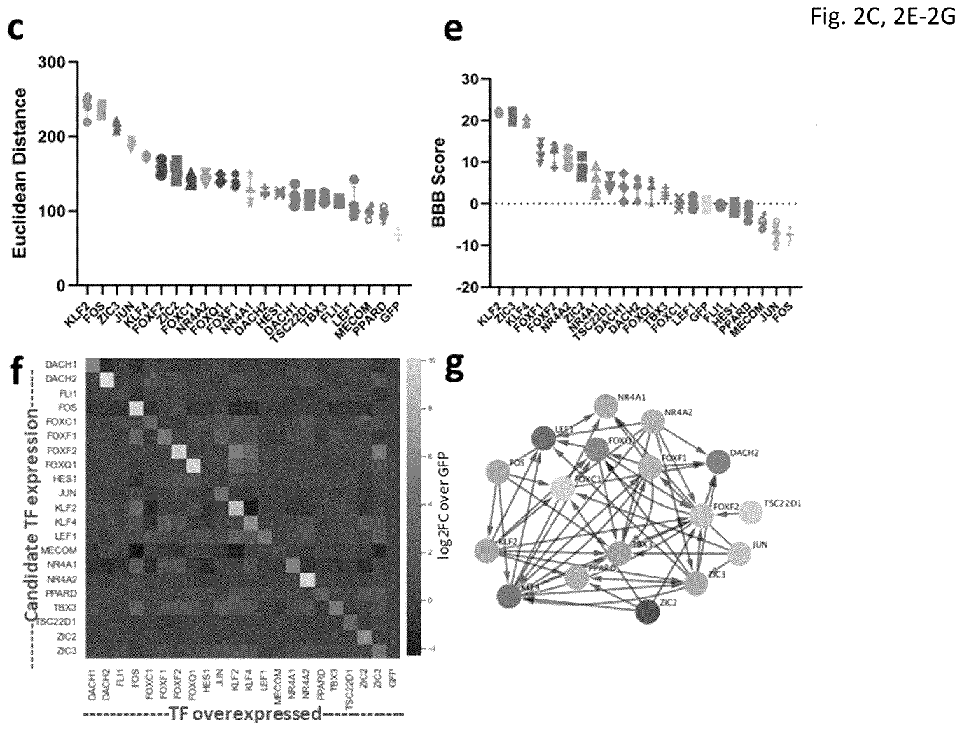

To quantify BBB gene expressions, we created a custom variable: the BBB score. It is defined as the sum of log 2 fold changes of the 88 BBB enriched genes (FIG. 2d). Since overexpression of TFs can increase or decrease the expression of these 88 genes, BBB score would capture both the benefits and disadvantages. However, to limit the influence of large fold change of a single gene on BBB score, we capped the influence from each of the 88 genes from −1 to 1. In summary, the BBB score is calculated as:

$$BBB Score = {\sum }_{i} ( log 2 FC over {GFP}_{Gene i} )$$

To predict combinations of TFs for capturing more in vivo BBB-like phenotype, we first calculate the change induced by each of the TF, and call the variable ΔTFᵢ. Then we used the pseudobulk function form the glmGamPoi package (Ahlmann-Eltze and Huber 2021), and obtained gene counts from the brain capillary and brain tip cell populations (Zarkada et al. 2021). We then calculate the difference between the capillary cell population and the tip cell calculation, and call the variable Δ in vivo. We then calculate the average of three, four, or five ΔTFᵢ profiles, and calculated the distance from the to average to Δ in vivo using the dist function from math package on Python (Van Rossum and Python Development Team 2018). We then rank the distances and the top predictions with smallest distances were tested experimentally.

### Immunocytochemistry

Cells were fixed with −20° C. methanol (Sigma, 67-56-1) or 4% paraformaldehyde (PFA; Electron Microscopy Sciences, 15700) in DPBS for 15 min at room temperature. Following three washes in DPBS the cells were blocked in 10% goat serum (Sigma, G9023) for 30 min. Cells were then incubated with primary antibodies at indicated dilution ratios at 4° C. overnight (Table 1). Cells were then washed with PBS three times. Cells were then incubated with secondary antibodies (Table 1) and 20 μM Hoechst 33342 (Thermo Scientific, 62249) for 1 hour at room temperature. Cells were then washed three times. For widefield fluorescence microscopy, we used a Nikon. For confocal microscopy, we treated samples with ProLong Gold Antifade (Life Technologies, P36941) and imaged using a Nikon A1R microscope. For quantification of immunocytochemistry images, background signals from a secondary antibody only control were deducted from all conditions.

TABLE 1
Antibodies used in this study

| | | | Dilution |
|---|---|---|---|
| Target | Fixation | Host species | for ICC |
| ABCB1 (C219) | MeOH | Mouse IgG1 | 1:100 |
| ABCB1 (F4) | MeOH | Mouse IgG1 | 1:100 |
| SLC2A1 (SA0277) | MeOH | Rabbit | 1:100 |
| ABCG2 (5D3) | MeOH | Mouse IgG2b | 1:100 |
| OCLN (OC-3F10) | MeOH | Mouse IgG1 | 1:100 |
| CAV1 | MeOH | Rabbit Poly | 1:500 |
| LSR | MeOH | Rabbit Poly | 1:100 |
| SLC38A5 | MeOH | Rabbit Poly | 1:100 |
| SLCO2B1 | MeOH | Rabbit | 1:100 |
| | | Monoclonal | |
| LEF1 | MeOH | Mouse IgG1 | 1:200 |

### RT-qPCR

RNA was extracted using Direct-zol RNA Miniprep kit (Zymo, R2050). Reverse transcription was performed to obtain cDNA using GoScript Reverse Transcriptase with Oligo (dT) kit (Promega, A2791). Real-time gene expression analysis was conducted using 25-μl reactions containing SYBR Green PCR Master Mix (Life Technologies, 4309155) along with primers specific for gene of interests (Table 2). PCR was run according to manufacturer protocols on Agilent AriaMX Real-Time PCR system.

TABLE 2
Primer sequences for RT-qPCR used in this study

| Target | Forward Primer | Reverse Primer |
|---|---|---|
| CDH5 | GAACCAGATGCACATTGATGA | TGCCCACATATTCTCCT |
| | AG | TTGAG |
| | (SEQ ID NO: 1) | (SEQ ID NO: 2) |
| PECAM1 | CAGGCCCCATTGTTCCC | ATTGCTCTGGTCACTTC |
| | (SEQ ID NO: 3) | TCC |
| | | (SEQ ID NO: 4) |
| CLDN5 | TGACCTTCTCCTGCCACTA | AAGCGAAATCCTCAGTC |
| | (SEQ ID NO: 5) | TGAC |
| | | (SEQ ID NO: 6) |
| OCLN | ATGGCAAAGTGAATGACAAGC | AGGCGAAGTTAATGGAA |
| | (SEQ ID NO: 7) | GCTC |
| | | (SEQ ID NO: 8) |
| ABCB1 | ACTCACTTCAGGAAGCAACC | GATTGACTGAATGCTGA |
| | (SEQ ID NO: 9) | TTCCTC |
| | | (SEQ ID NO: 10) |
| ABCG2 | CTCAGATCATTGTCACAGTCG | GTCGTCAGGAAGAAGAG |
| | T | AACC |
| | (SEQ ID NO: 11) | (SEQ ID NO: 12) |
| SLC2A1 | GTGCCATACTCATGACCATCG | GGCCACAAAGCCAAAGA |
| | (SEQ ID NO: 13) | TG |
| | | (SEQ ID NO: 14) |
| SLC7A5 | TCCATCCTCTCCATGATCCAC | GTTGAAGAAGCTGAAGA |
| | (SEQ ID NO: 15) | AGTTGATG |
| | | (SEQ ID NO: 16) |
| SLC38A5 | GCCATGTCCAGTTACCTGTT | TTTCCCTTCAAGAACCA |
| | (SEQ ID NO: 17) | GTCC |
| | | (SEQ ID NO: 18) |
| MFSD2A | GCATCCTCCAAAGCACTGAA | CAAGTGCATAGCAAAGC |
| | (SEQ ID NO: 19) | TTGT |
| | | (SEQ ID NO: 20) |
| CAV1 | CATGGCAGACGAGCTGAG | AAACTGTGTGTCCCTTC |
| | (SEQ ID NO: 21) | TGG |
| | | (SEQ ID NO: 22) |
| PLVAP | TGGACACCTGCATCAAGAC | GGATCTTCCTCTTGAAC |
| | (SEQ ID NO: 23) | TCCTC |
| | | (SEQ ID NO: 24) |

### Dextran and Albumin Endocytosis Assays

We used two different fluorescently-labeled tracers previously reported to be associated with caveolae-mediated endocytosis: Alexa Fluor 647-conjugated albumin (Invitrogen A34785) and Alexa Fluor 488-conjugated 10 kDa dextran (Invitrogen D22910). Dextran was added at 10 μM to the medium of Passage 1 cultures. Plates were incubated on rotating platforms at 37 or 4° C. for 2 hr. Medium was removed and cells were washed once with DPBS, and then incubated with Accutase for 10 min at 37° C. Cell suspensions were passed through 40 μm cell strainers into 4× volume of DMEM/F-12 and centrifuged for 5 min, 200×g. Pellets were resuspended in MACS buffer and analyzed on a Attune flow cytometer (ThermoFisher). FlowJo software was used to analyze flow cytometry results.

### Results

### Mining Single Cell Transcriptomics Data to Identify BBB-Enriched Transcription Factors

To identify TFs with enriched expression in BBB endothelial cells, we performed independent analyses of single cell transcriptomics datasets. In the first analysis, we compared the transcriptomes of murine brain EC populations with murine ECs from five peripheral vascular beds: heart, lung, liver, kidney and skeletal muscle (Kalucka et al. 2020), generating a list of 48 TFs enriched in murine brain EC populations (FIG. 1). For the second analysis, we pooled human brain ECs from five different single cell transcriptomics datasets (Polioudakis et al. 2019; La Manno et al. 2016; Zhong et al. 2020; Han et al. 2020; Hodge et al. 2019), and compared brain EC gene expression to pooled human heart, liver, lung and skeletal muscle ECs (Aizarani et al. 2019; Travaglini et al. 2020; Cui et al. 2019; Rubenstein et al. 2020) to identify 55 TFs with enriched expression in human brain EC populations (FIG. 1), in a similar approach previously performed by our group (Gastfriend, Foreman, et al. 2021). Finally, we compared non-barrier forming brain tip cells with capillary endothelial cells that have acquired a transcriptomic barrier signature in a human brain single cell transcriptomics dataset (Crouch et al. 2022). Enriched TFs in each comparison with log 2 fold change larger than 1, and with p value less than 0.05 in Wilcoxon rank sum test were selected. From this integrated analysis, we identified 40 TFs that exhibited increased expression during barriergenesis in human capillary ECs. The three independent analyses generated sets of BBB-enriched TFs having high similarity, with 40 out of the 92 TFs enriched in at least two out of the three datasets and 11 TFs enriched in all three comparisons (FIG. 1).
To narrow the list of candidate TFs, we transduced hPSC-derived endothelial progenitor cells with individual TFs and assessed their effects on a panel of BBB transcripts. To this end, hPSCs were differentiated through mesoderm progenitors to EPCs as previously described reported method (Bao et al. 2015; Gastfriend, Nishihara, et al. 2021). After EPC purification by CD31+ magnetic-activated cell sorting (MACS) lentiviruses delivering each of the 44 candidate TFs were dosed onto EPCs along with treatment of Wnt agonist CHIR99021 (FIG. 2a). Resultant fpCECs harboring TFs or control lentivirus were evaluated by RT-qPCR for a panel of 8 genes enriched at the BBB (CLDN5, OCLN, ABCB1, ABCG2, SLC2A1, SLC7A5, SLC38A5, MFSD2A), 2 genes downregulated at the BBB (CAV1, PLVAP), and 2 endothelial cell markers (CDH5, PECAM1) (FIG. 1C). We found that out of the 44 tested TFs, many induced some transcriptional aspects of BBB character. For example, KLF2 and KLF4 significantly increased the expression of MFSD2A; ZIC2, ZIC3, KLF2 and KLF4 significantly increased the expression of SLC38A5 and KLF2 significantly increased the expression of ABCB1 (FIG. 1C). Based on the RT-qPCR screen, we selected TFs that are capable of inducing BBB-like gene expressions in the correct direction, and narrowed the list of candidate TFs to 21 candidate TFs for comprehensive transcriptomic characterization (FIG. 1)
BBB-Enriched TFs Induce BBB-Like Gene Expression in fpCECs
Given that the initial qPCR screen indicated that a subset of TFs could drive interesting discrete changes in BBB gene expression, we assessed the global transcriptomic changes elicited by overexpression of individual TFs using the same experimental setup (FIG. 2A). Bulk RNA sequencing analysis was performed on fpCECs individually transduced with each of the 21 TFs. CECs transduced with a GFP overexpression lentivirus were used as controls. A principal component analysis (PCA) of all sequenced transcriptomic profiles revealed that gene expression induced by overexpression of KLF family TFs dominated PC1, while overexpression of AP-1 family TFs FOS and JUN dominated PC2 (FIG. 2B). FOXF1, FOXF2 and ZIC3 also induced significant changes in gene expression (FIG. 2B). The observation is confirmed when we calculated the Euclidean distances between transcriptomic profiles to the average of GFP controls, and found that KLF2, FOS, ZIC3, JUN, KLF4 and FOXF2 induced the most significant global gene expression changes (FIG. 2C). To specifically evaluate the impact of TF overexpression on BBB specification, we curated a list of 88 genes that were upregulated in murine brain ECs compared to peripheral ECs in a single cell sequencing study, while still having significant expression levels in human brain EC datasets (Table 3, details in Methods). We visualized the effect of the 21 TFs on expression of the 88 BBB genes and found that although no single TF induced all 88 BBB genes, KLF2 and KLF4, FOXF1 and FOXF2, ZIC3, NR4A1 and NR4A2, FOXQ1 each induced fairly discrete BBB gene subsets (FIG. 2D). To quantify these changes, we calculated a BBB score to measure the collective extent of TF overexpression on BBB transcriptional program induction (details in Methods) and identified that KLF2 and KLF4, ZIC2 and ZIC3, FOXF1 and FOXF2, NR4A2 and NR4A1 had the highest BBB score, indicating their capability to induce BBB-like gene expression changes in fpCECs (FIG. 2E). We then probed whether any of the 21 TFs can regulate the expression of other BBB-enriched TFs and identified significant crosstalk (FIG. 2F). For example, overexpression of KLF2 induced the expression of LEF1, FOXF2, FOXQ1 and ZIC3 while overexpression of ZIC3 induced the expression of FOXF2, FOXQ1, KLF4 and LEF1 (FIG. 2F). These relationships were also summarized in a STRING plot (FIG. 2G), which identified KLF family TFs, ZIC family TFs, FOX family TFs and NR4A family TFs to be the TFs most capable of increasing the expression of other BBB-enriched TFs.

TABLE 3
BBB-enriched genes

ABCB1
ABCG2
ABLIM1
ACVRL1
AFAP1L2
ALAS1
ALPL
APOD
APOLD1
ARL4A
BSG
Clorf54
CA2
CA4
CCDC141
CD320
CLDN5
DEGS2
DUSP1
EDN1
EDN3
ELOVL7
EOGT
FLT1
FN1
FOXF2
FOXQ1
FRY
GLUL
GPCPD1
HBA1
HBA2
HSPA1A
IFNAR1
IGF1R
ITIH5
ITM2A
JUN
JUND
KLF2
LEF1
LSR
MAOA
MAPILC3A
MFSD2A
MPZL1
MT1E
NEBL
NKD1
NOSTRIN
NR4A1
OCLN
PAQR5
PKIG
PLAT
PLLP
PLTP
PRNP
PROM1
PTN
RASD1
SGMS1
SLC16A1
SLC16A2
SLC16A4
SLC2A1
SLC31A1
SLC38A3
SLC38A5
SLC39A10
SLC3A2
SLC40A1
SLC7A1
SLC7A5
SLCO1A2
SLCO2B1
SORBS2
SPOCK2
ST3GAL6
STMN1
TDRP
TFRC
TOB1
TPD52L1
TSC22D1
TSPAN5
VWA1
ZIC3

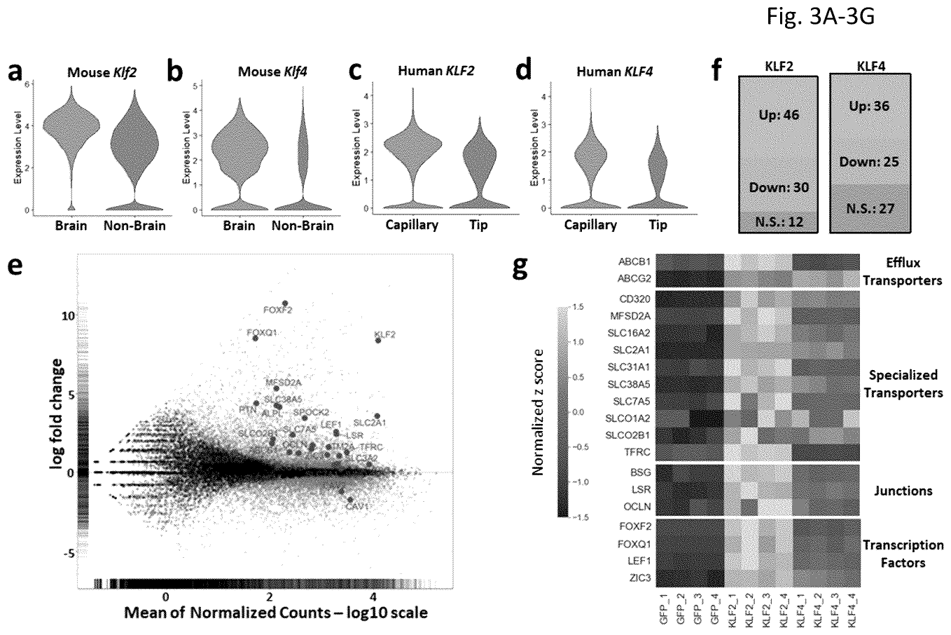

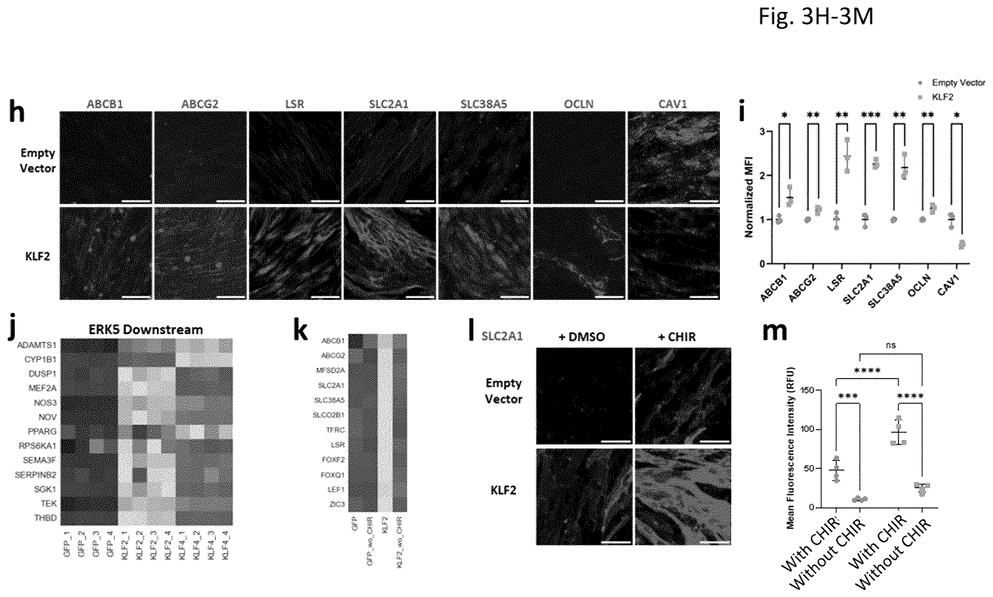

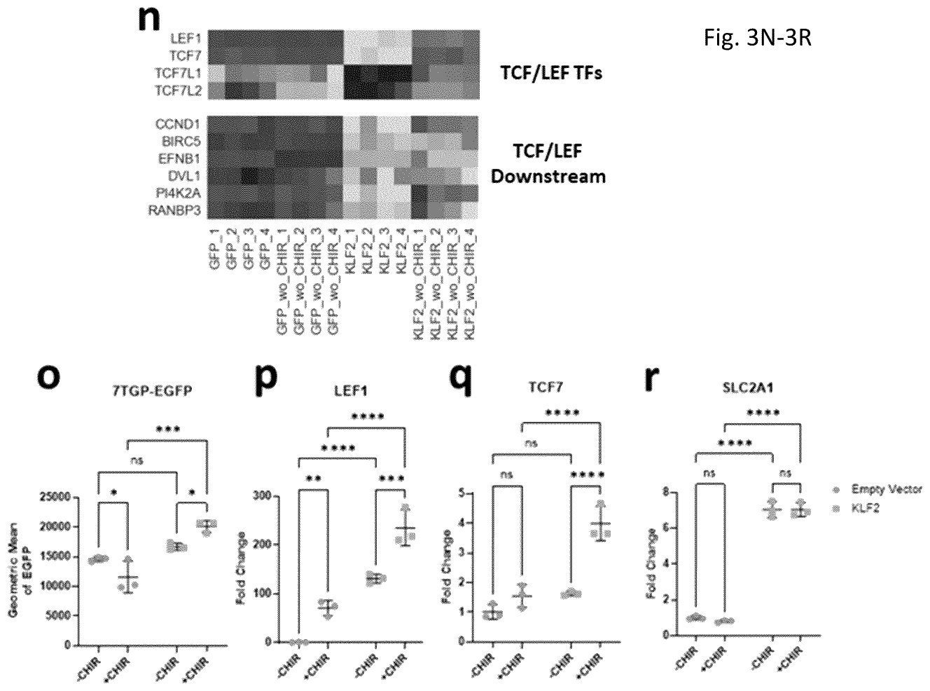

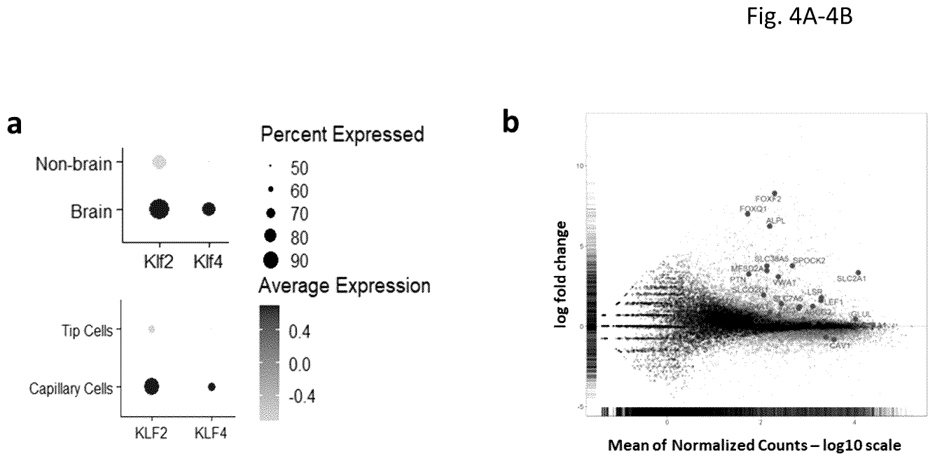

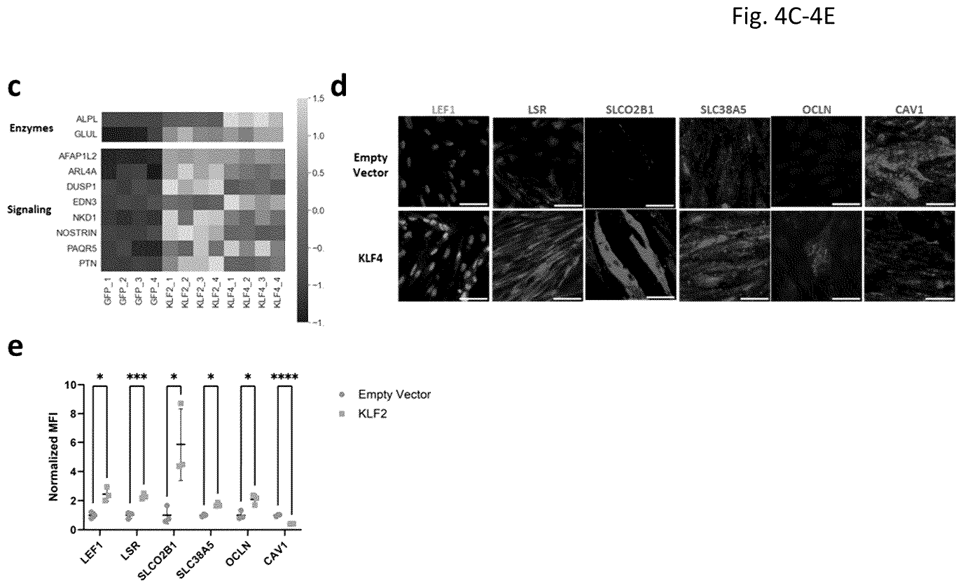

KLF2 and KLF4 Induce BBB-Like Gene Expression in fpCECs
KLF2 and KLF4, zinc finger proteins belonging to the Kruppel family of TFs, play important roles in mammalian development (Pollak et al. 2018). Specifically in endothelial cells, shear stress induces MEK5/ERK5 signaling, which in turn increases KLF2 and KLF4 expression (Paudel, Fusi, and Schmidt 2021). KLF family TF expression can also regulate endothelial gene expression (Sweet et al. 2023; Tsaryk et al. 2022), increase chromatin accessibility (Moonen et al. 2022), and regulate vascular inflammation and vasoprotective effects (Sweet et al. 2018). Both KLF2 and KLF4 overexpresssion increased the BBB score in fpCECs. In single cell transcriptomics datasets, Klf2 and Klf4 were upregulated in murine brain endothelial cells compared to non-brain endothelial cells (FIG. 3A, 3B). In addition, KLF2 and KLF4 were upregulated in human brain capillary cells compared to tip cells (FIG. 3C, 3D). When analyzing the effect of KLF2 or KLF4 expression in fpCECs, we found that out of the 88 BBB-enriched genes, KLF2 increased the expression of 46 of the 88 BBB-enriched genes and decreased the expression of 30 genes. Similarly, KLF4 increased the expression of 36 and decreased the expression of 25 of these 88 BBB-enriched genes. (FIG. 3F). Specifically, we found that KLF2 or KLF4 overexpression increased the expression of BBB-enriched TFs, including FOXF2, FOXQ1 and LEF1 at relatively high fold changes, indicating that they may serve as upstream regulators for several key BBB-enriched TFs (FIG. 3E, FIG. 4B). KLF2 or KLF4 overexpression also significantly induced the expression of efflux transporter transcripts ABCB1 and ABCG2, specialized transporter or transporter-related transcripts MFSD2A, SLC16A2, SLC2A1, SLC31A1, SLC38A5, SLC7A5, SLCO1A2, SLCO2B1 and TFRC, and adhesion and junction-related genes BSG, LSR and OCLN (FIG. 3G). KLF2 or KLF4 overexpression also increased the expression of several BBB-enriched enzyme and signaling-related transcripts (FIG. 4C). KLF2 or KLF4 overexpression reduced the expression of CAV1 and CAV2 transcripts, key molecular components of caveolae-mediated endocytosis (FIG. 3E). We validated transcript-level changes by probing a subset of protein targets via immunocytochemistry (ICC), and found that on a protein level, KLF2 overexpression significantly increased the expression of ABCB1, ABCG2, LSR, SLC2A1, SLC38A4 and OCLN, while significantly decreasing the protein expression of CAV1 (FIG. 3H, 3I). Similarly, KLF4 overexpression increased protein expression of LEF1, LSR, SLCO2B1, SLC38A5, OCLN, and decreased protein expression of CAV1 (FIG. 4D, 2E). While several proteins were upregulated in all transduced cells, others like OCLN were upregulated in a subset of transduced cells. Since it has been suggested that KLF2 and KLF4 are targets of ERK5 signaling (Paudel, Fusi, and Schmidt 2021), we analyzed the expression of genes downstream of ERK5 signaling (Qiagen GeneGlobe databse), and found that KLF2 or KLF4 overexpression significantly increased the expression of such genes (FIG. 3J).

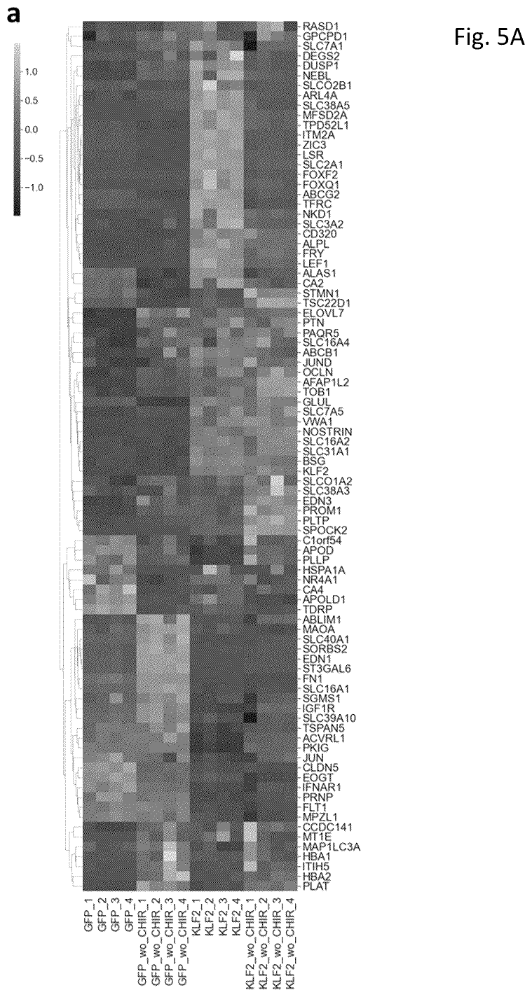

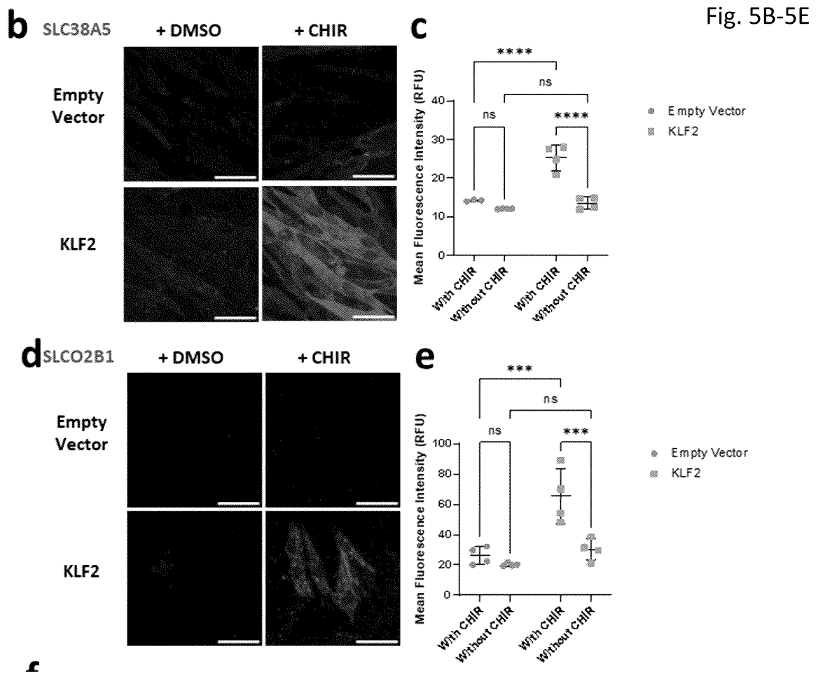

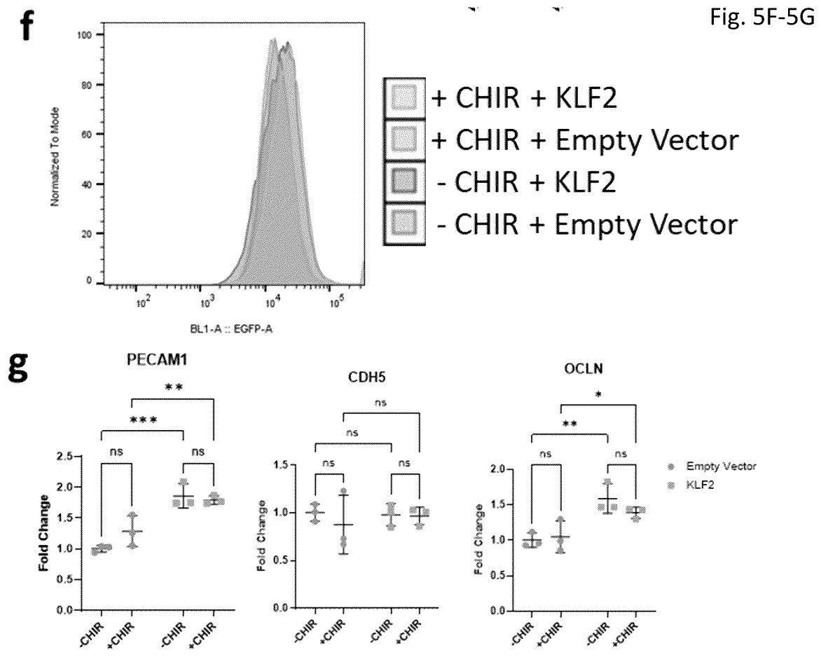

In the hPSC-derived CEC system, concordant with TF transduction and overexpression, Wnt signaling is activated by supplementing the Wnt agonist CHIR99021 (FIG. 2A). Recognizing the strong effect of KLF2 or KLF4 overexpression on BBB gene expression, explored whether activated Wnt signaling impacted the ability of KLF overexpression to modulate BBB gene expression. We therefore sequenced both the GFP-transduced control and KLF2 overexpression samples without CHIR99021 exposure. Several key BBB genes, including ABCB1, ABCG2, MFSD2A, SLC2A1, SLC38A5, SLCO2B1, TFRC, LSR, FOXF2, FOXQ1, LEF1 and ZIC3 were only upregulated in samples having both KLF2 overexpression and Wnt activation by CHIR99021 (FIG. 3K, FIG. 5A). This finding indicates a synergistic effect of these two pathways in inducing BBB gene expression. Further validation at the protein level indicated that the induction of protein expression of SLC2A1, SLC38A5 and SLCO2B1 induced by KLF2 overexpression, required Wnt co-activation (FIG. 3L, 3M, FIG. 5B, 5C, 5D, 5E). Since several of the genes synergistically induced by KLF2 overexpression and CHIR99021 treatment are known targets of canonical Wnt signaling, such as SLC2A1 (K. Li et al. 2025; Ding et al. 2025; Gastfriend, Nishihara, et al. 2021), LEF1 (Cadigan and Waterman 2012) and SLC38A5 (Z. Wang et al. 2022), we analyze the expression of several key TCF/LEF TFs and downstream targets. We found that combining KLF2 overexpression with CHIR99021 treatment significantly increased the expression of canonical Wnt signaling activators LEF1/TCF7 and reduced the expression of Wnt inhibitory TFs TCF7L1 and TCF7L2 (FIG. 3N). We also observed a higher expression of downstream TCF/LEF target genes in samples treated with both CHIR99021 and KLF2 overexpression (FIG. 3N). Such shifts in Wnt TF expression have been reported to be correlated with β-catenin levels, and represents higher canonical Wnt activities (Guo et al. 2021). Thus, to further assess the canonical Wnt signaling activation, we used an hPSC Wnt reporter line, H9 7TGP. The engineered 7TGP line expresses GFP under the control of a consensus TCF/LEF binding sequence promoter that reports canonical Wnt/β-catenin signaling activation (Lian et al. 2012; Fuerer and Nusse 2010). After differentiating H9 7TGP hPSCs to endothelial progenitors (FIG. 2A), they were treated with KLF2 lentivirus and/or CHIR99021. We found that while KLF2 overexpression alone did not induce GFP expression in H9 7TGP CECs, KLF2 overexpression in conjunction with CHIR99021 treatment significantly induced GFP expression over CHIR99021 treatment alone (FIG. 3O, FIG. 5F), indicating stronger canonical Wnt signaling activity with combined KLF2 and CHIR99021 treatment. To assess whether these factors induce BBB gene expression in primary EC, we treated. HUVECs with KLF2 lentivirus and CHIR99021. This treatment significantly increased the expression of canonical Wnt signaling TF transcripts LEF1 and TCF7, while the expression of BBB markers SLC2A1 and OCLN were only upregulated in the KLF2 treated HUVECs (FIG. 3P-R, FIG. 5G). Synergistic treatment did not affect expression of endothelial markers PECAM1 and CDH5 in HUVECs (FIG. 5G).

Taken together, we identified that KLF2 and KLF4 TFs induced significant BBB-like gene and protein expression. Interestingly, these effects were significantly more pronounced when KLF overexpression is accompanied by Wnt activation. In addition, the expression of key BBB-enriched TFs, including FOXF2, FOXQ1 and ZIC3 were only significantly upregulated when both KLF overexpression and Wnt activation are both present.

### FOX and ZIC Family Transcription Factors Induce BBB-Like Gene Expression

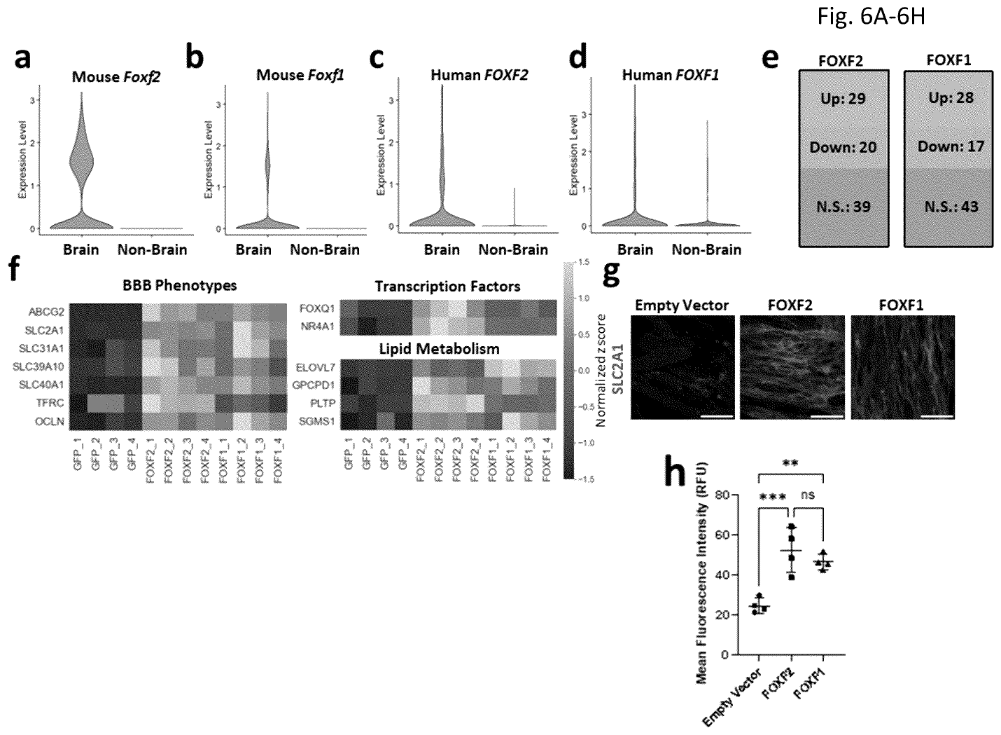

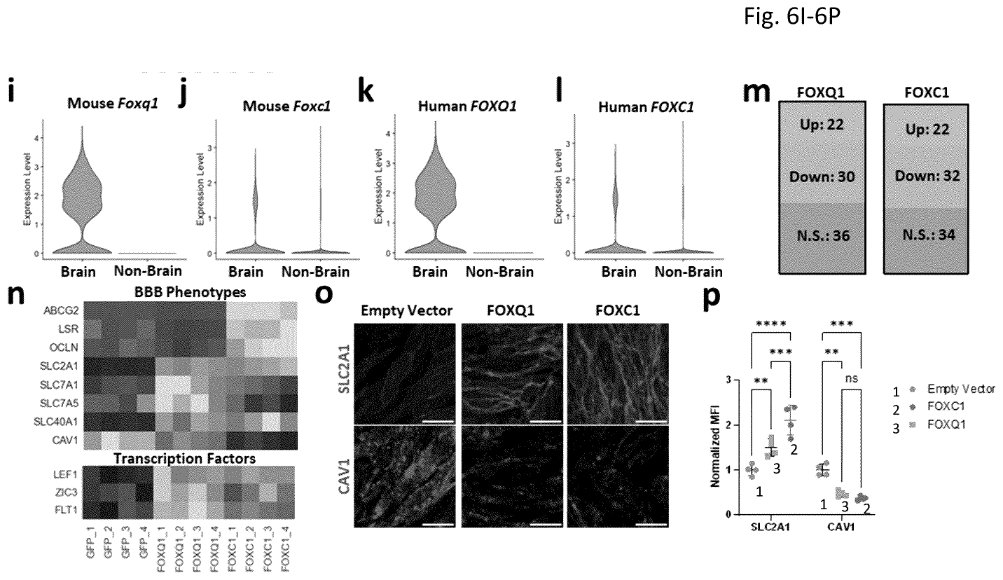

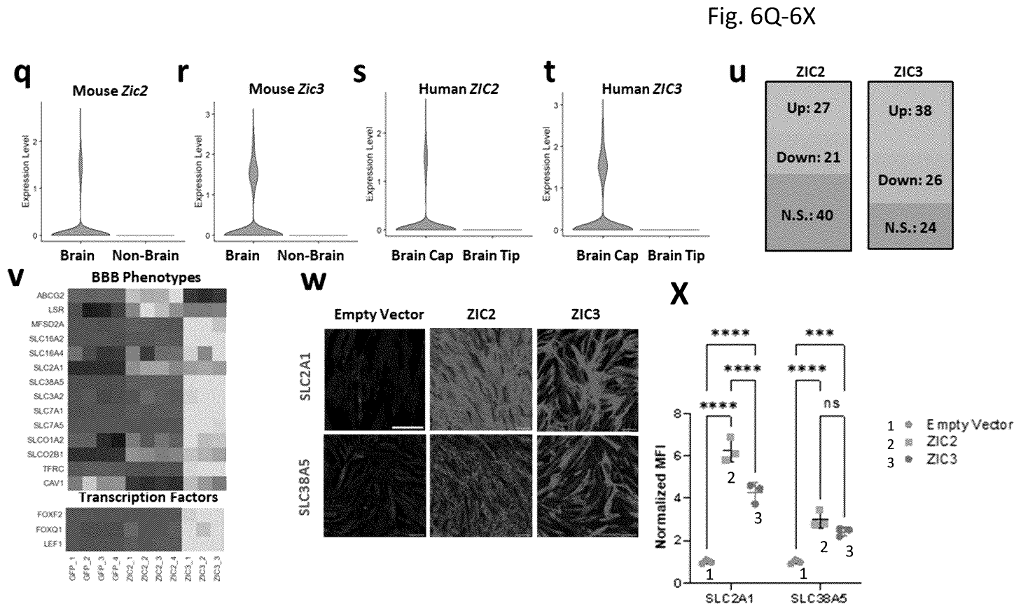

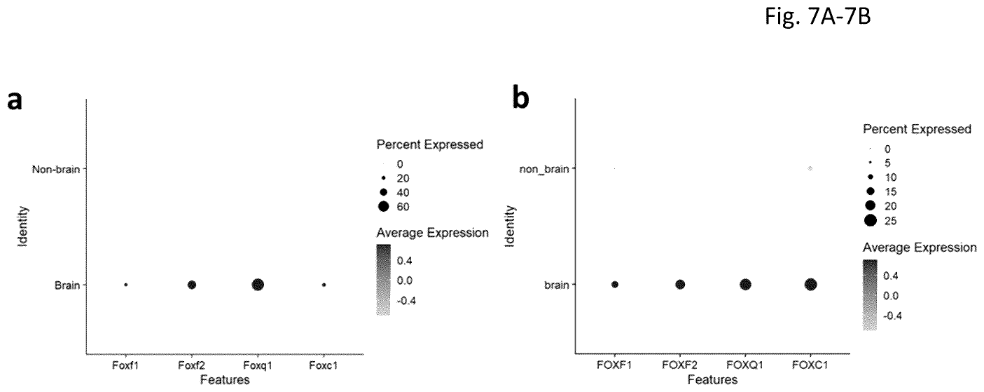

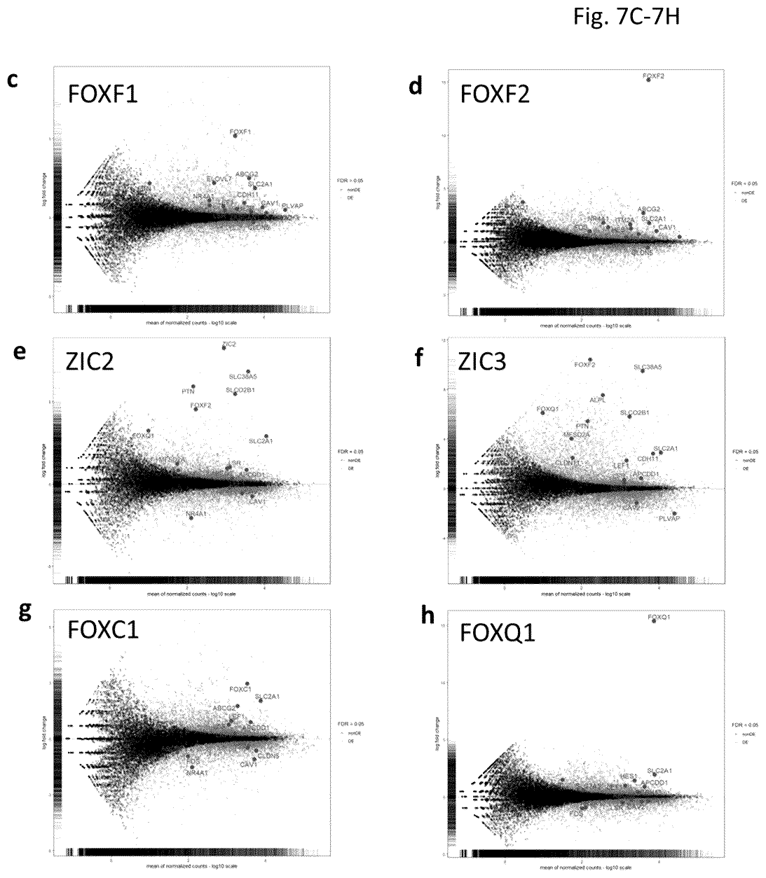

Forkhead box (Fox) transcription factors regulate biological processes during development (Golson and Kaestner 2016). Overexpression of FOXF1, FOXF2, FOXQ1, FOXC1 expression improved BBB scores in fpCECs. Notably, the expression of these four TFs is almost absent in non-brain EC populations, indicating their BBB vascular specificity (FIG. 6A-D, 4I-L, FIG. 7A, 7B). When analyzing the specific effects of FOXF1 or FOXF2 on BBB-enriched genes in fpCECs, we found that FOXF1 or FOXF2 overexpression increased the expression of BBB-enriched TFs FOXQ1 and NR4A, suggesting synergistic regulation amongst these TFs (FIG. 6E-F). We also found that FOXF1 or FOXF2 overexpression significantly induced the expression of efflux transporter ABCG2, specialized transporters SLC2A1, SLC31A1, SLC39A10, SLC40A1, TFRC and junctional gene OCLN and genes involved in lipid metabolism (FIG. 6F) and validated that both FOXF1 and FOXF2 induced SLC2A1 (glut-1) protein expression (FIG. 6G-H).

From PCA analysis, we found that overexpression of FOXC1 and FOXQ1 generated similar transcriptomic profiles (FIG. 2B). When analyzing the effect of FOXQ1 or FOXC1 in our hPSC-derived CEC system, we found that FOXC1 or FOXQ1 overexpression increased the expression of BBB-enriched TFs LEF1, ZIC3 and FLT1 (FIG. 6M-N). We also found that FOXC1 overexpression significantly induced the expression of transporter genes ABCG2 and SLC2A1, tight junction genes LSR and OCLN while FOXQ1 overexpression more prominently induced the collective expression of specialized transporters SLC2A1, SLC7A1, SLC7A5 and SLC40A1 (FIG. 6N, FIG. 7C-D). Both FOXC1 and FOXQ1 reduced the expression of CAV1. Concordant with the transcript level changes, protein expression of SLC2A1 was significantly increased and CAV1 was significantly decreased by FOXQ1 and FOXC1 overexpression (FIG. 6O-P).

ZIC2 and ZIC3 overexpression also significantly enhanced the BBB score (FIG. 2e). ZIC family TFs are zinc finger proteins that play important roles in pluripotency and nervous system development (Hatayama and Aruga 2018). Notably, the expression of ZIC2 and ZIC3 are almost absent in non-brain EC populations, indicating specificity for the BBB (FIG. 6Q-T). When analyzing the effect of ZIC2 or ZIC3 in our hPSC-derived CEC system, we found that ZIC3 induced more significant BBB-like changes than ZIC2 (FIG. 2E, 6U-V, FIG. 7E-F). Notably, ZIC3 overexpression increased the expression of BBB-enriched TFs FOXF2, FOXQ1 and LEF1 (FIG. 6V). We also found that ZIC3 overexpression significantly induced the expression of BBB-enriched genes MFSD2A, SLC16A2, SLC16A4, SLC2A1, SLC38A5, SLC3A2, SLC7A1, SLC7A5, SLCO1A2, SLCO2B1 and TFRC (FIG. 6V). We validated that protein expression of SLC2A1 and SLC38A5 increased with either ZIC2 or ZIC3 overexpression (FIG. 6W, 4X). Overall, we found that FOX family and ZIC family TFs can induce significant BBB-like character in fpCECs expressions.

### Combinatorial Expression of BBB-Enriched TFs Enhances BBB-Like Gene Expression

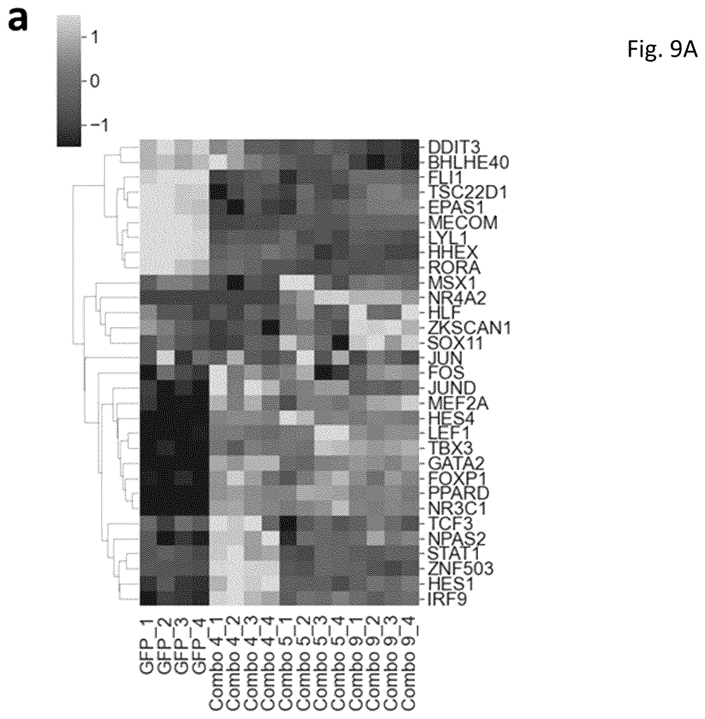

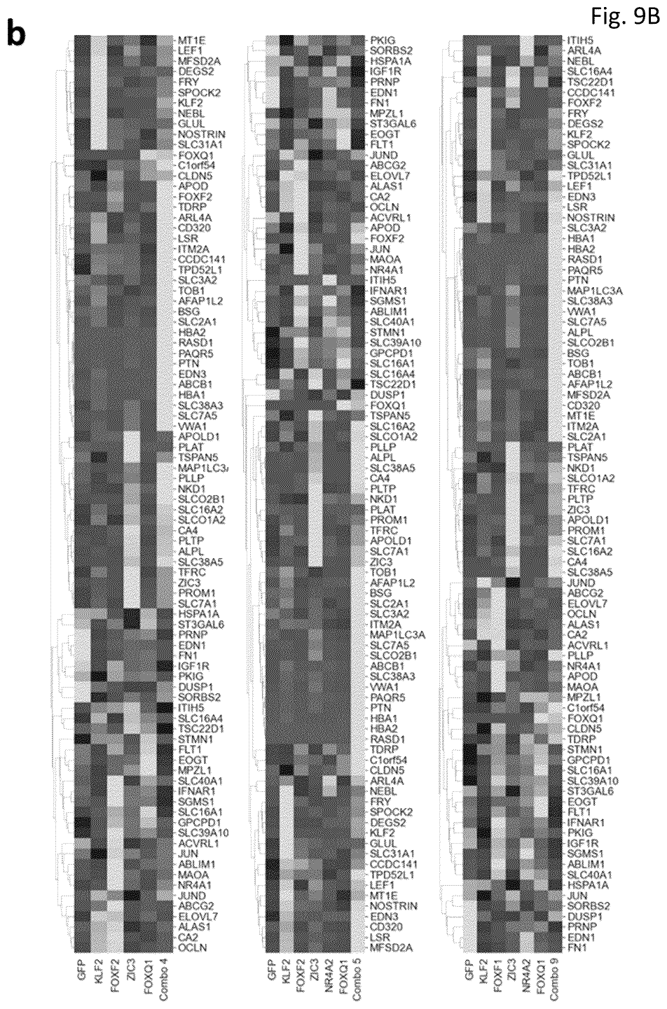

Realizing that overexpression of single TFs induced distinct subsets of the BBB gene expression program (FIG. 2D), we hypothesized that combining the expression of multiple transcriptional regulators would achieve a broader recapitulation of BBB gene expression. To test this, we calculated the average predicted transcriptional profile changes when overexpressing multiple TF combinations and determined how closely these predicted transcriptomic shifts compared to the changes observed between non-barrier tip cells and capillary endothelial cells. In other words, we specifically endeavored to predict and compare the transcriptome shifts elicited by TF overexpression to those observed during barriergenesis in vivo (Details in methods). An averaged transcriptomic profile approach was chosen based on findings that averaging transcriptomics profiles outperform other methods in predicting results on combinatorial TF overexpression (Joung et al. 2024). Eleven of the top predictions of TF combinations were then chosen to test experimentally (FIG. 8A). When compared to BBB gene expression changes upon single TF overexpression (FIG. 2D), expression of a combination of TFs lead to a broader set of BBB gene induction (FIG. 8B, compare to GFP control). Multiple TF overexpression was confirmed for each TF combo (FIG. 8C). Principal component analysis delineated impact of KLF family TF overexpression along the diagonal of PC1 and PC2, and ZIC3 overexpression along PC2, with each combination leading to distinct transcriptomes from those generated by overexpression of any single TF (FIG. 8D). We again performed the BBB Score analysis to quantify enrichment of BBB genes and found that all 11 TF combinations tested gave higher BBB scores than any single TF (FIG. 8E), indicating combinatorial BBB-inducing impact for these lead TF candidates. Notably, the TF combinations with top BBB scores (Combo 9, 4 and 5) exhibited smaller overall transcriptomic Euclidean distances away from GFP control, compared to KLF2 (FIG. 8F), suggesting a selective impact of these TFs on BBB gene expression rather than just reflecting a global shift in gene expression. We then assessed the expression of the 40 BBB-enriched TFs shown in FIG. 1 and found that the top TF combinations (Combo 4, 5, 9) were able to further induce expression of a majority of the BBB-enriched TFs, suggesting that these discrete 4-5 member TF combinations have the potential to initiate the broader BBB-specification transcriptional program (FIG. 9A). When comparing the BBB-enriched gene expression elicited by TF combinations with that generated by overexpressing the individual TFs that make up the combo, we found that subsets of the BBB-enriched gene expression profiles are shared between individual TF and TF combination samples as expected. There were also many instances where TF combinations resulted in significantly enhanced BBB-enriched gene expression compared with that resulting from individual TFs (FIG. 9B). Moreover, there were also instances where TF combinations reduced the level of induction compared to single TF overexpression. These observations suggest synergistic interplay amongst these TFs in inducing BBB-like gene expression.

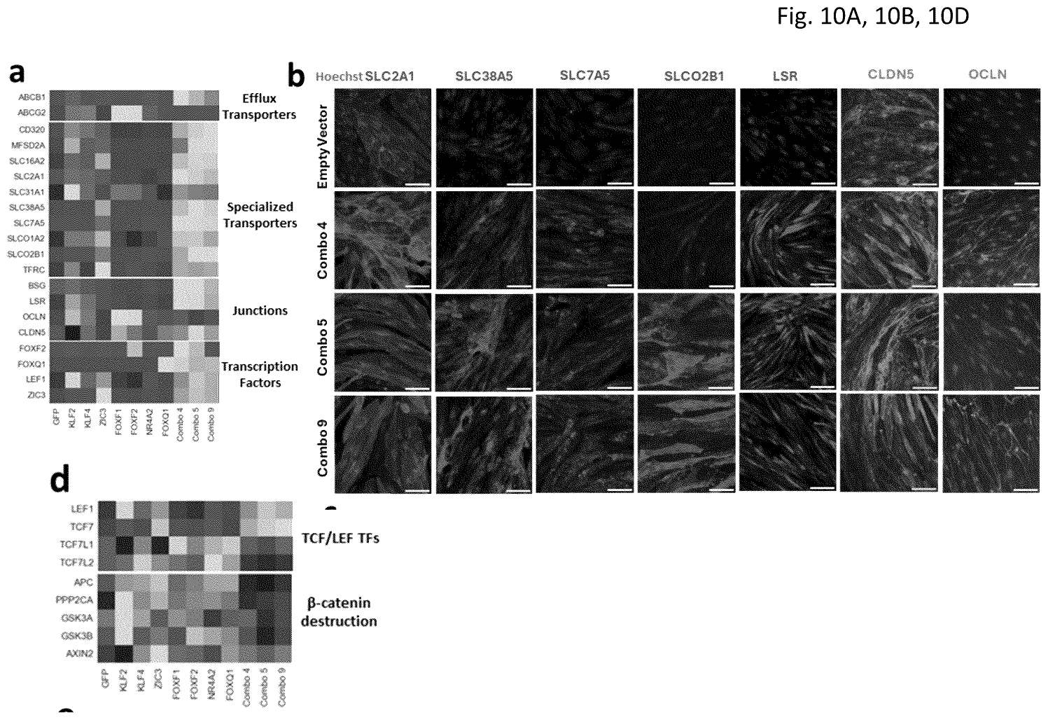

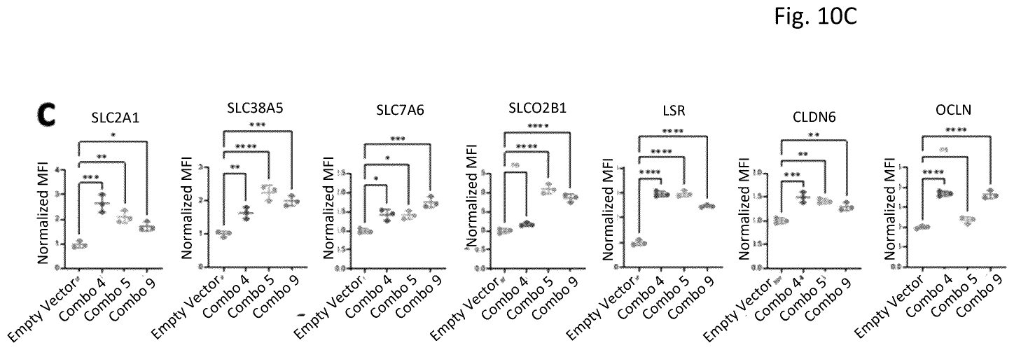

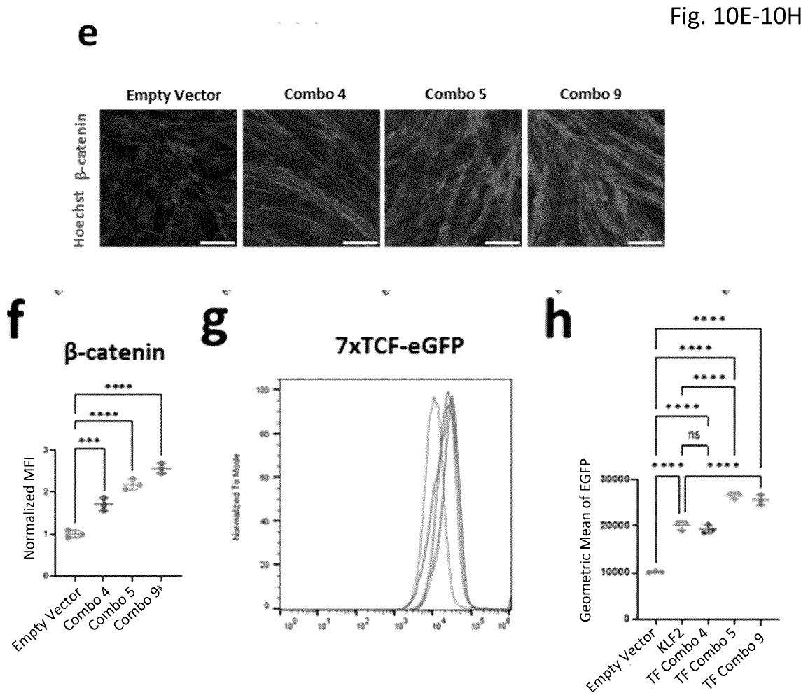

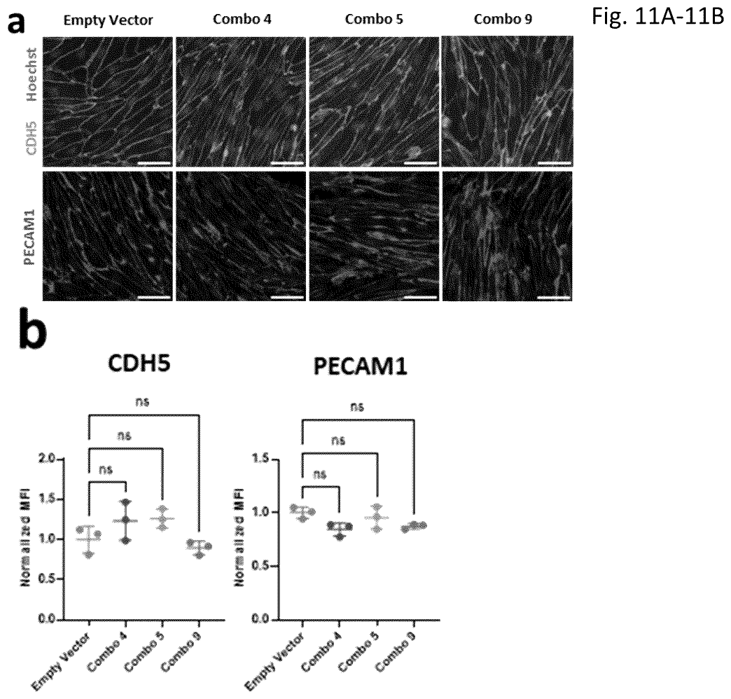

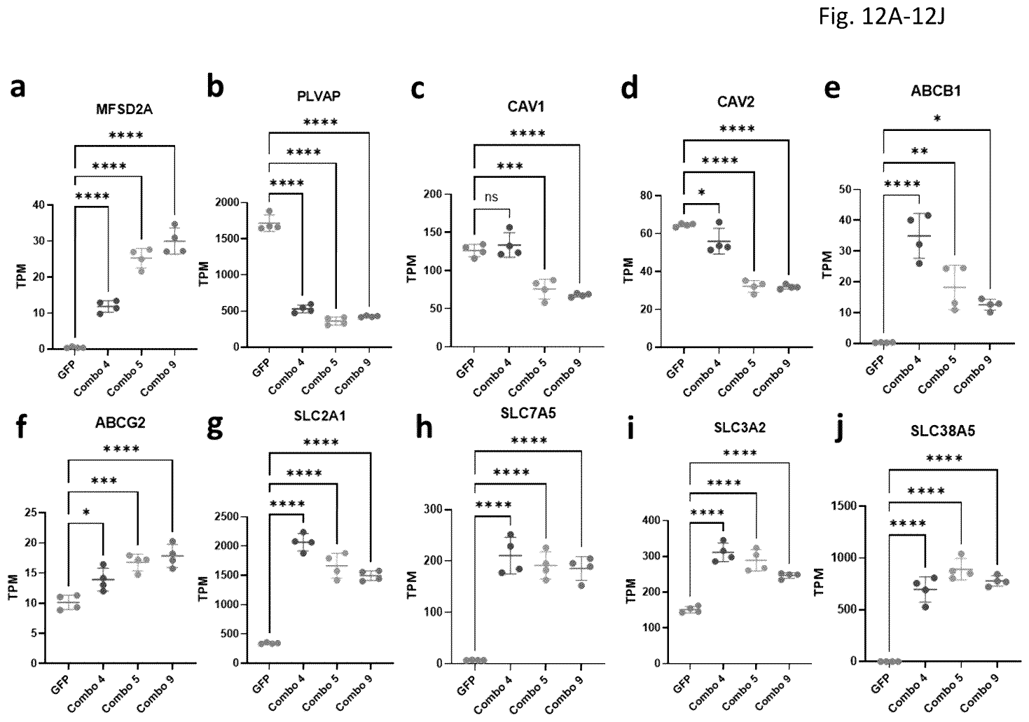

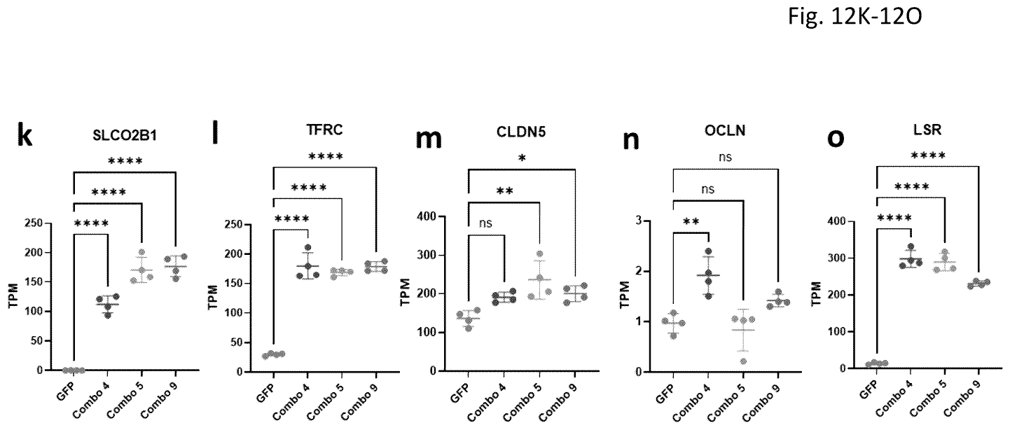

When examining BBB-like gene expression induced by TF combinations, we found that the top combinations (Combo 4,5,9) induced the expression of efflux transporter ABCB1 above that observed for any single TF, while ABCG2 was induced above GFP controls, albeit at lower levels than those observed for some single TFs. A variety of solute carrier (SLC) transporters and junctional genes (BSG, LSR, and CLDN5) were also induced by top combinations (Combo 4,5,9) (FIG. 10A, FIG. 12). We verified elevated protein expression for a subset of these genes, SLC2A1, SLC38A5, SLC7A5, CLDN5 and LSR for TF combinations 4 and 9 (FIG. 6B-C). The fpCECs overexpressing TF combinations 4 and 9 also retained expression of endothelial markers CDH5 and PECAM1, indicating an unchanged cell fate (FIG. 11). As with KLF2 alone, the TF combinations resulted in an induction of Wnt activating TFs LEF1 and TCF7, and inhibition of Wnt inhibitory TFs TCF7L1 and TCF7L2. However, compared with individual TFs, we observed a more pronounced decrease in the expression of genes responsible for β-catenin destruction (APC, PPP2CA, GSK3A and GSK3B) (FIG. 10D). It is known that stabilizing β-catenin and reducing expression of β-catenin destruction genes is linked to higher levels of canonical Wnt signaling (Stamos and Weis 2013; MacDonald, Tamai, and He 2009). Thus, to confirm our transcriptomic observations, we determined that β-catenin protein levels were increased in fpCECs overexpressing either TF combination 4 or 9 (FIG. 10E-F). Next, it was determined that canonical Wnt signaling was increased by TF combinations 4, 5 or 9 in the 7TGP hPSC line, with combinations 5 and 9 further increasing Wnt signaling over KLF2 overexpression alone. (FIG. 10G-H). These data again suggest that further activated canonical Wnt signaling is an important driver of the acquisition of BBB character in fpCECs.

### Forward Programmed CNS-Like ECs Demonstrate BBB-Related Phenotypes

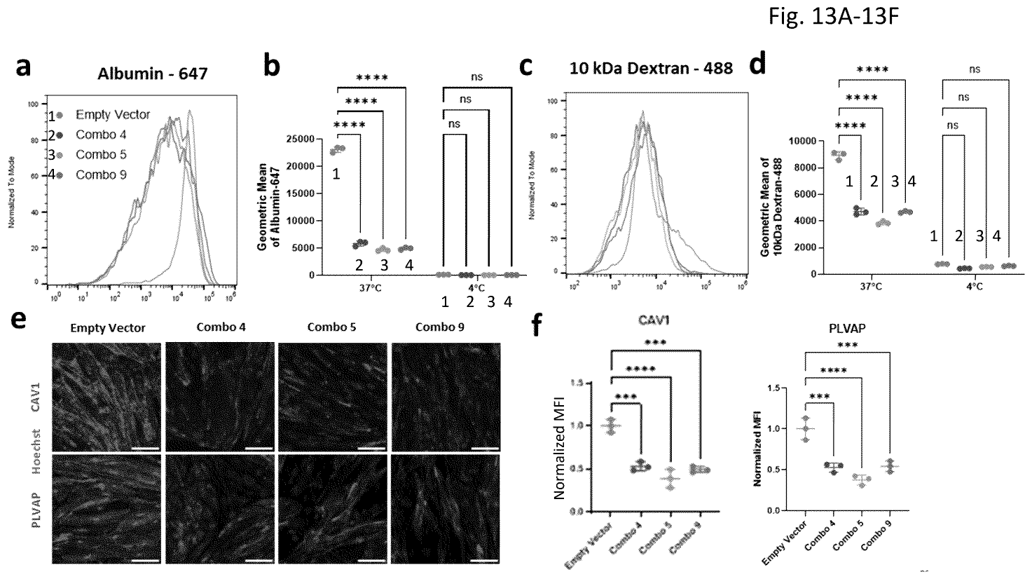

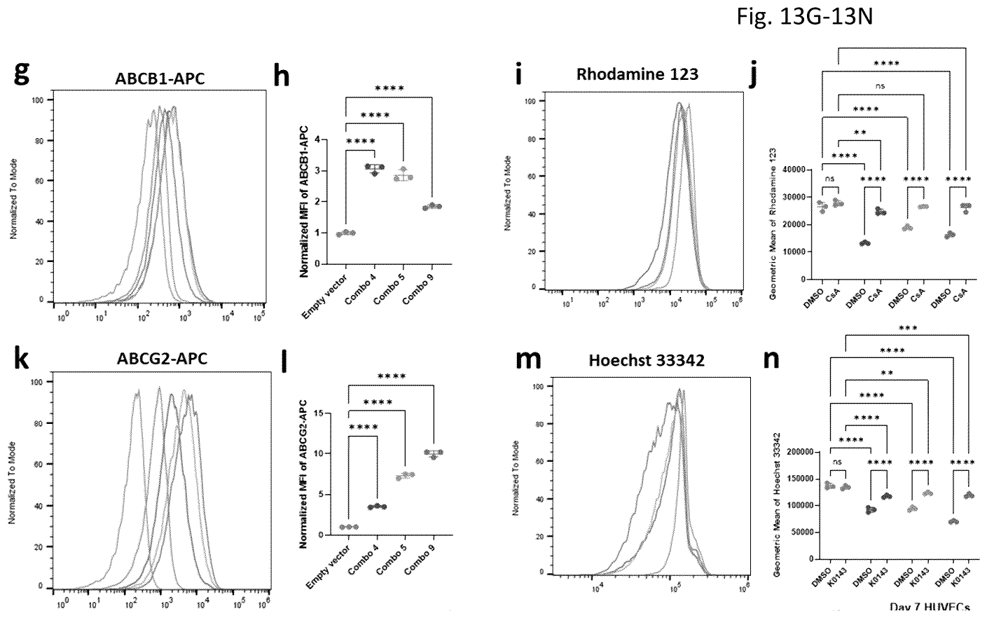

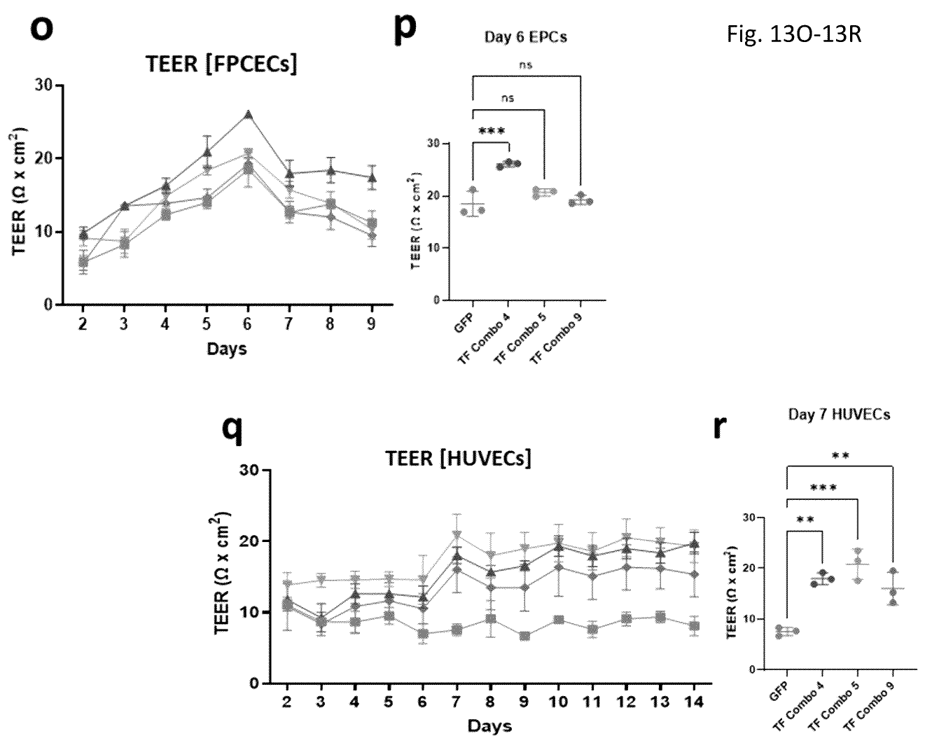

With promising gene and protein expression data in fpCECs overexpressing TF combinations (FpCECs), we next examined whether these molecular attributes manifested in three main BBB phenotypes; reduced vesicular trafficking, efflux transporter activity and passive barrier formation. Reduction of caveolae-mediated endocytosis is a hallmark of BBB ECs that correlates with reduced CAV1 expression (Zhao, Song, and Zhang 2014; Andreone et al. 2017; Chow et al. 2020). TF combination 4 and 9 fpCECs have significantly reduced CAV1 expression (FIG. 12C, FIGS. 13E-F) and they were evaluated for their endocytosis capabilities by measuring the uptake of fluorescently-labeled albumin and 10 kDa dextran, which both have been suggested to be driven by caveolae-mediated endocytosis pathways (Chatterjee et al. 2017; H.-H. Li et al. 2013; Andreone et al. 2017; L. Li et al. 2015). We found that for all three combinations of TFs tested (Combo 4, 5 and 9), we observed significant decrease in intracellular accumulation of both albumin and 10 kDa dextran at 37° C. (FIG. 13A-D). By contrast there was no difference observed at 4° C., a condition limiting membrane fluidity and inhibiting endocytosis. Together, these data suggested that fpCECs demonstrate reduced caveolae-mediated uptake, fluid phase endocytosis.

Another key feature of BBB ECs is efflux transporter activity with ABCB1 (p-glycoprotein) and ABCG2 (BCRP) playing key roles. Concordant with transcriptional increases in ABCB1 where ABCB1 expression was turned on by TF combination overexpression from a baseline TPM near 0 (FIG. 12E), flow cytometry indicated significant induction of cell surface ABCB1 protein expression (FIG. 13G-H). To evaluate whether the increase in expression corresponds to efflux function, we measured the accumulation of Rhodamine123 (Rh123), a substrate of p-glycoprotein in fpCECs with or without P-gp inhibitor cyclosporin A (CsA). We observed that the fpCECs had reduced Rh123 accumulation in the cells as measured by flow cytometry (FIG. 13I-J), indicating that these cells actively effluxed Rh123 in the absence of inhibitor CsA. By contrast, when the fpCECs were treated with CsA, the decrease in Rhodamine123 accumulation was abrogated to the level of the control CECs (FIG. 13J). The fpCECs also possessed increased cell surface ABCG2 protein expression, that correlated with transcript levels (FIG. 13K,L, FIG. 12F). To verify its functionality, Hoechst 33342 was used as the ABCG2 substrate, and a reduction in Hoechst accumulation was observed in fpCECs in the absence of ABCG2 inhibitor Ko143 (FIG. 13M). However, when the fpCECs were treated with Ko143, increased Hoechst 33342 accumulation was observed (FIG. 13N). Together, these data indicate that whereas control CECs possessed no detectable efflux activity, fpCECs possess robust ABCB1 and BCRP activity.

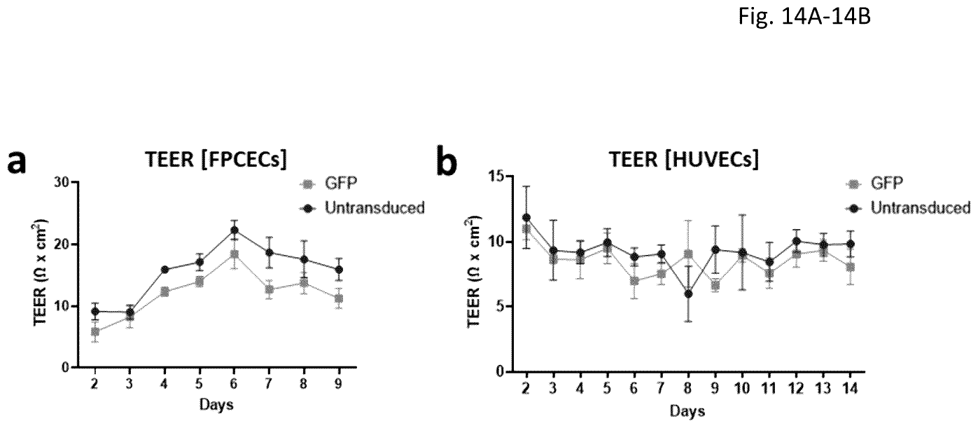

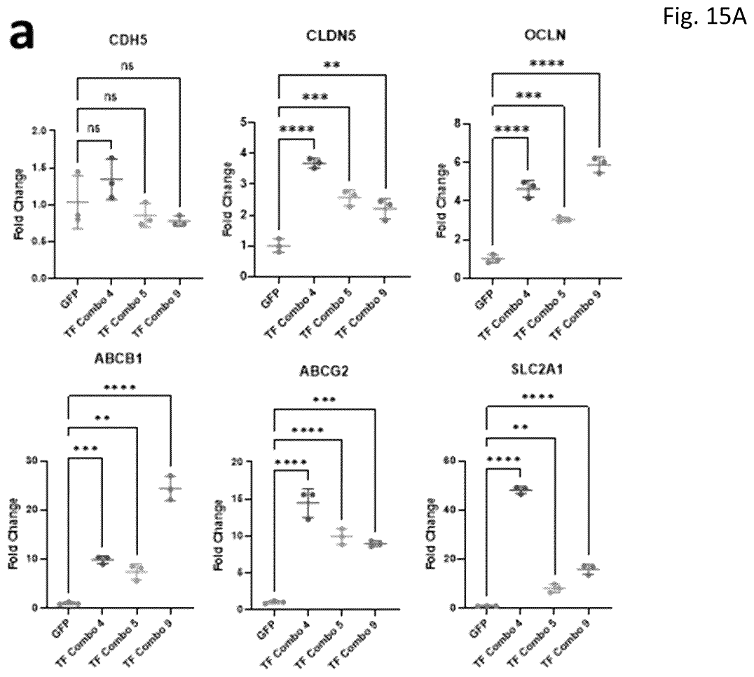

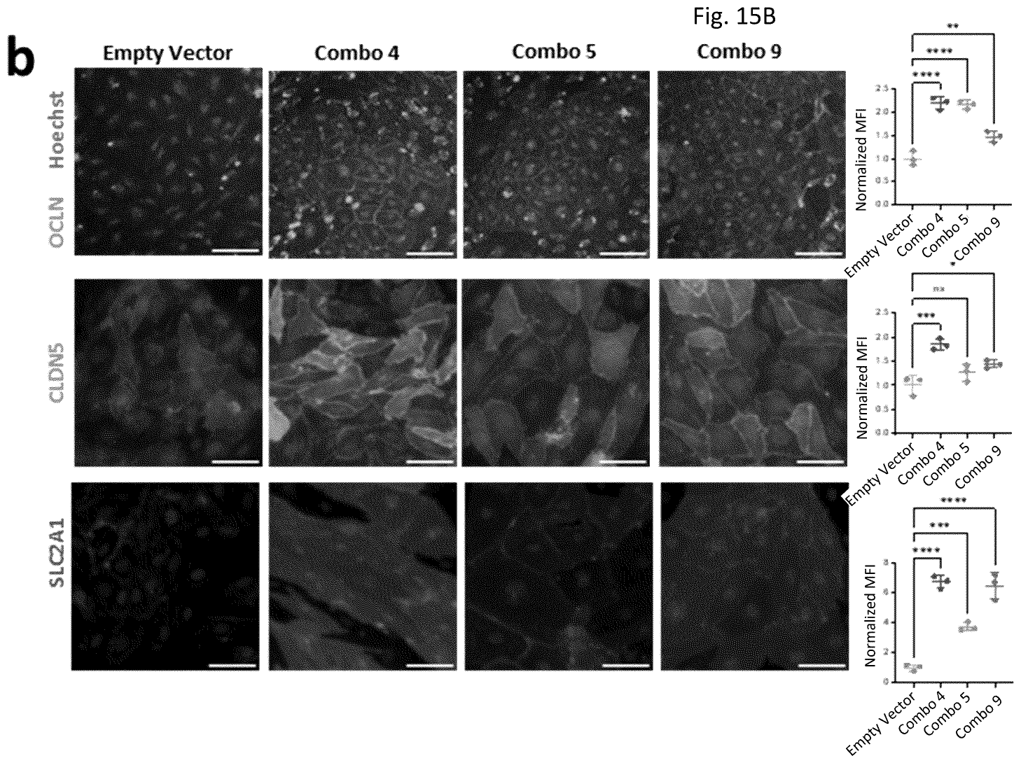

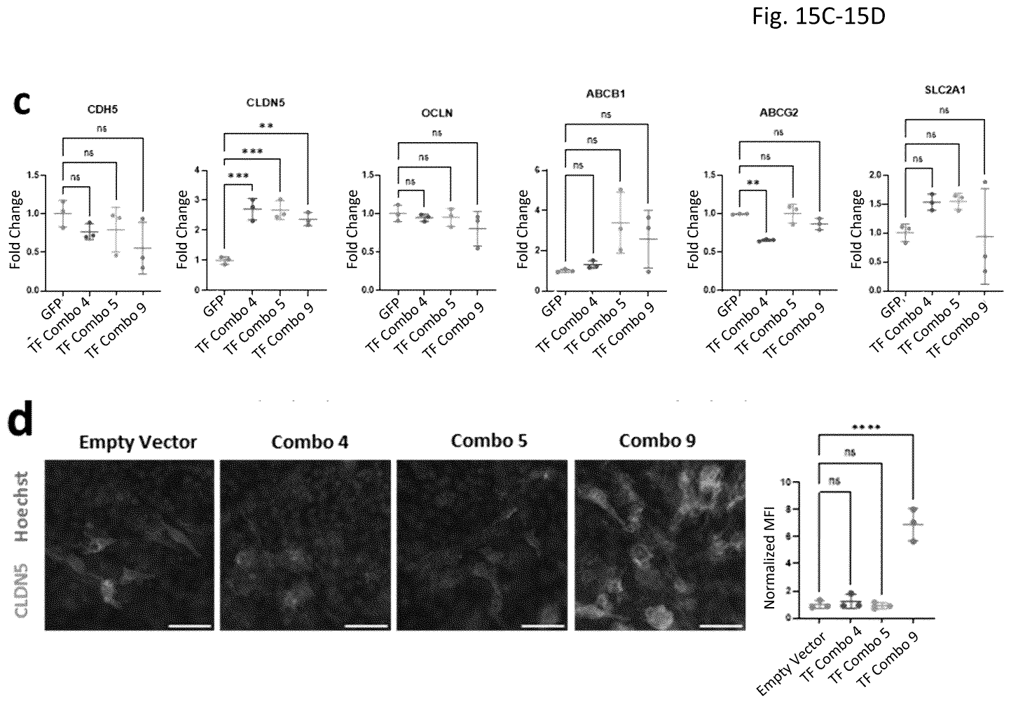

The third key BBB property measured was tight junction barrier formation using transendothelial electrical resistance (TEER). We observed that the fpCECs with TF combination 4 had a modest increase in TEER over the CEC control (FIG. 13O-P). Since lentiviral transduction itself has a deleterious impact on TEER (FIG. 14A), we sought to test another human endothelial cell source for its TEER response to lentivirally administered TF combinations. We therefore tested HUVECs and determined that lentiviral transduction did not seem to impact baseline TEER as observed with the CECs (FIG. 14A-B). Thus, we transduced HUVECS with the TF combinations and all 3 combinations led to increased TEER (FIG. 13Q, R). This increase could be attributed to the increase CLDN5 protein expression along with the appearance of OCLN protein expression (FIG. 15A, B). Given that the TF combinations improved barrier properties in HUVECs, we also explored the impacts on a subset of other related transcripts. TF combinations also increased transcript levels of SLC2A1, ABCB1, and ABCG2 in HUVECs (FIG. 15B). As we saw induction of BBB-related gene and protein expression in HUVECs through overexpression of TF combinations, we next evaluated whether the TF combinations would rescue to the loss of barrier phenotype in immortalized human brain endothelial cell line hCMEC/D3. We evaluated gene expression changes induced by the TF combinations 4, 5, and 9, and observed that they increased CLDN5 expression (FIG. 15C). Upon protein expression analysis, the CLDN5 expression was seen to be increased by TF combination 9 (FIG. 15D). Altogether, these data suggest that the TF combinations are potent in induction of barrier phenotypes in non-brain endothelial cell lines such as HUVECs and may potentially play a role in rescuing the loss of barrier phenotype in immortalized brain endothelial cell line hCMEC/D3.

### Discussion

The blood-brain barrier (BBB) is a highly specialized interface essential for maintaining brain homeostasis and protecting it against exogenous substances (Daneman and Prat 2015). As reviewed in chapter 1, in vitro models of the BBB possess high value, as they can be used for disease modeling, drug permeability screening, and developmental biology studies. Among the types of in vitro models, hPSC-derived BBB models stand out in terms of unlimited cell source, high scalability and high barrier properties. In the last decade, advancements have been achieved to differentiate BBB endothelial cells from pluripotent stem cell sources. Notably, the unconditioned medium (UM) method encouraged hPSC differentiation to allow ectodermal lineage cells and endothelial populations to co-differentiate. Endothelial populations were then selectively expanded and purified (Lippmann et al. 2012). The subsequent exposure to retinoic acid significantly induced barrier properties of these iPSC-derived BBB ECs (Stebbins et al. 2018; Lippmann et al. 2014). These models have been extensively explored in applications including disease modeling (Katt et al. 2016), shear modulation (Vatine et al. 2019) and drug permeability screens (Ohshima et al. 2019). Albeit superior barrier qualities, these models have been recently found to have a mixed endothelial and epithelial cell fate signature, and does not capitulate the gene expression of in vivo BBB cells closely (Lu et al. 2021). Recently, research efforts have been focusing on patterning hPSC-derived definitive endothelial cells or endothelial progenitor cells to acquire BBB EC-like gene expression and phenotypes. Notably, it has been found that Wnt activation by CHIR99021 drives acquisition of some BBB EC-like gene expression (Gastfriend, Nishihara, et al. 2021). Inhibition of TGFβ signaling by RepSox increased expression of Claudin-5 (Roudnicky et al. 2020). A combination of Wnt activation by lithium chloride, inhibition of Inhibition of TGFβ signaling by A8301 and activation of cAMP signaling drive acquisition of some BBB EC-like gene expression and phenotypes (Porkoláb et al. 2024). However, none of these methods were able to generate cells with high barrier quality quantified by TEER, and BBB-like gene expressions are generally modest. This is an indication that foundational basic biology knowledge on BBB development is still lacking, and the true inducers for BBB cell fate remain to be identified.

To combat these short-comings, this study adopts a comprehensive search and forward programming approach. Namely, we identified TFs that are enriched at the BBB through single cell transcriptomics analysis and tested the effect of overexpressing these TFs on hPSC-derived EPCs. We found that KLF2, KLF4, FOXF1, FOXF2, ZIC2, ZIC3, NR4A1, NR4A2, DACH1, DACH2, FOXC1 or FOXQ1 overexpression could induce subsets of BBB-like gene expressions. Among the lead candidate TFs, KLF2 and KLF4 TFs stand out because they are able to induce expression of other BBB-enriched TFs, suggesting that they might be master regulators of BBB cell fate. Besides, KLF2 and KLF4, compared to other TFs, induced most significant BBB-like gene expression. We also found that KLF2 and KLF4 overexpression, in synergy with Wnt activation, stimulates BBB-like gene expression. Analysis of signaling pathway revealed that this synergy is driven by a switch from TCF7L1/TCF7L2 expression to LEF1/TCF7 activation, indicating a strong induction of canonical Wnt signaling, the most important pathway suggested by literature in BBB development (Liebner et al. 2008). This finding is especially interesting due to the role of KLF2 and KLF4 in physiology. For endothelial cells, it has been established in literature that shear stress induced KLF2 and KLF4 expression (Tsaryk et al. 2022; Doddaballapur et al. 2015). Shear stress is established when nascent capillaries start to carry laminar blood flow, and is considered to be a crucial factor for endothelial physiology commonly missed in static cell culture (Y.-S. J. Li, Haga, and Chien 2005). Shear stress cannot be the sole contributor for BBB-like gene expression because while shear stress is prevalent for endothelial cells throughout different organs, the barrier phenotype of ECs is only available at the BBB or brain-retina barrier. We explored this by coupling activation of canonical Wnt signaling into the picture because CNS and retina cell types actively secret Wnt ligands at large amount, including Wnt7a and norrin (Daneman et al. 2009; Seitz et al. 2010). Our results suggest that coupling Wnt activation with shear stress-induced KLF family TF overexpression might be driving BBB cell fate acquisition. Future animal and in vitro mechanistic studies are needed to further understand this newly discovered regulation. We also analyzed the effects of other BBB-enriched TFs, including FOX family and ZIC family TFs in detail. We found that these TFs are also considered as downstream targets of canonical Wnt signaling. Although their effects are not as significant as KLF family TFs, they are also able to induce subsets of BBB-like gene expression.

Realizing that overexpression of different TFs individually overexpresses different subsets of BBB-like gene expression, we tested overexpression of combinatorial TFs to capture a more comprehensive BBB-like gene expression. Using computational predictions, we identified several combinations of TFs. After testing them experimentally, we found that these combinations are capable of synergistically inducing comprehensive BBB-like gene expression. Specifically, combinations of KLF2 or KLF4 with FOXF2, ZIC3, NR4A2, and FOXQ1 enhanced the expression of 62 out of 88 canonical BBB markers. We also found that these fpCECs have gene expression profiles remarkably similar to in vivo BMECs and demonstrate reduced endocytosis capabilities. Compared to existing in vitro BBB models derived from stem cells through small molecules and growth factors, our forward programmed model displays superior scalability and captures a much broader spectrum of BBB gene expression and functionalities.

We envision that future engineering efforts are needed to make these fpCECs better suited as in vitro BBB models. In this study, we used lentivirus to deliver constitutive expression of TFs to the EPCs. This approach allowed for rapid screening to identify candidate TFs but failed to provide a more precise dose and timing control for TF overexpression. We envision that utilizing an inducible promoter approach, similar to strategies adopted in Chapter 3 of this manuscript, will be allow for dose and timing control of TF overexpression, a crucial factor for ensuring homogeneity of fpCECs. In addition, we envision future studies on higher level signaling modulation on KLF family TFs, including overexpression of ERK5 signaling components, small molecule and growth factor inducers for ERK5 signaling, and application of shear stress to capture effects on induction of BBB properties.

### REFERENCES

Ahlmann-Eltze, Constantin, and Wolfgang Huber. 2021. “GlmGamPoi: Fitting Gamma-Poisson Generalized Linear Models on Single Cell Count Data.” Bioinformatics (Oxford, England) 36 (24): 5701-2.
Aizarani, Nadim, et al. 2019. “A Human Liver Cell Atlas Reveals Heterogeneity and Epithelial Progenitors.” Nature 572 (7768): 199-204.
Andreone, Benjamin J., et al. 2017. “Blood-Brain Barrier Permeability Is Regulated by Lipid Transport-Dependent Suppression of Caveolae-Mediated Transcytosis.” Neuron 94 (3): 581-594.e5.
Bao, Xiaoping et al. 2015. “Chemically-Defined Albumin-Free Differentiation of Human Pluripotent Stem Cells to Endothelial Progenitor Cells.” Stem Cell Research 15 (1): 122-29.
Bestor, T. H. 2000. “Gene Silencing as a Threat to the Success of Gene Therapy.” The Journal of Clinical Investigation 105 (4): 409-11.
Bolger, Anthony M., Marc Lohse, and Bjoern Usadel. 2014. “Trimmomatic: A Flexible Trimmer for Illumina Sequence Data.” Bioinformatics (Oxford, England) 30 (15): 2114-20.
Bray, Sarah J. 2006. “Notch Signalling: A Simple Pathway Becomes Complex.” Nature Reviews. Molecular Cell Biology 7 (9): 678-89.
Butler, Andrew, et al. 2018. “Integrating Single-Cell Transcriptomic Data across Different Conditions, Technologies, and Species.” Nature Biotechnology 36 (5): 411-20.
Cadigan, Ken M., and Marian L. Waterman. 2012. “TCF/LEFs and Wnt Signaling in the Nucleus.” Cold Spring Harbor Perspectives in Biology 4 (11): a007906-a007906.
Chatterjee, Moumita, et al. 2017. “Caveolae-Mediated Endocytosis Is Critical for Albumin Cellular Uptake and Response to Albumin-Bound Chemotherapy.” Cancer Research 77 (21): 5925-37.
Chow, Brian W., et al. 2020. “Caveolae in CNS Arterioles Mediate Neurovascular Coupling.” Nature 579 (7797): 106-10.
Crouch, Elizabeth E., et al. 2022. “Ensembles of Endothelial and Mural Cells Promote Angiogenesis in Prenatal Human Brain.” Cell 185 (20): 3753-3769.e18.
Csatári, Júlia, Heinz Wiendl, and Matthias Pawlowski. 2024. “Forward Programming Human Pluripotent Stem Cells into Microglia.” Trends in Cell Biology, May.
Cui, Yueli, et al. 2019. “Single-Cell Transcriptome Analysis Maps the Developmental Track of the Human Heart.” Cell Reports 26 (7): 1934-1950.e5.
Daneman, Richard, et al. 2009. “Wnt/β-Catenin Signaling Is Required for CNS, but Not Non-CNS, Angiogenesis.” Proceedings of the National Academy of Sciences of the United States of America 106 (2): 641-46.
Daneman, Richard, and Alexandre Prat. 2015. “The Blood-Brain Barrier.” Cold Spring Harbor Perspectives in Biology 7 (1): a020412.
Ding, Yunfeng, Eric V. Shusta, and Sean P. Palecek. 2021. “Integrating in Vitro Disease Models of the Neurovascular Unit into Discovery and Development of Neurotherapeutics.” Current Opinion in Biomedical Engineering 20 (December).
Ding, Yunfeng, et al. 2025. “ETV2 Overexpression Promotes Efficient Differentiation of Pluripotent Stem Cells to Endothelial Cells.” Biotechnology and Bioengineering, March.
Dobin, Alexander, et al. 2013. “STAR: Ultrafast Universal RNA-Seq Aligner.” Bioinformatics (Oxford, England) 29 (1): 15-21.
Doddaballapur, Anuradha, et al. 2015. “Laminar Shear Stress Inhibits Endothelial Cell Metabolism via KLF2-Mediated Repression of PFKFB3.” Arteriosclerosis, Thrombosis, and Vascular Biology 35 (1): 137-45.
Fuerer, Christophe, and Roel Nusse. 2010. “Lentiviral Vectors to Probe and Manipulate the Wnt Signaling Pathway.” PloS One 5 (2): e9370.
Garcia, Francisco J., et al. 2022. “Single-Cell Dissection of the Human Brain Vasculature.” Nature 603 (7903): 893-99.
Gastfriend, Benjamin D., Koji L. Foreman, Moriah E. Katt, Sean P. Palecek, and Eric V. Shusta. 2021. “Integrative Analysis of the Human Brain Mural Cell Transcriptome.” Journal of Cerebral Blood Flow and Metabolism: Official Journal of the International Society of Cerebral Blood Flow and Metabolism 41 (11): 3052-68.
Gastfriend, Benjamin D., et al. 2021. “Wnt Signaling Mediates Acquisition of Blood-Brain Barrier Properties in Naïve Endothelium Derived from Human Pluripotent Stem Cells.” ELife 10 (November).
Golson, Maria L., and Klaus H. Kaestner. 2016. “Fox Transcription Factors: From Development to Disease.” Development (Cambridge, England) 143 (24): 4558-70.
Gu, Jinchao, Brett Cromer, and Huseyin Sumer. 2021. “Forward Programming of Pluripotent Stem Cells to Neurons.” Current Molecular Medicine 21 (1): 5-14.
Guo, Qiuyu, et al. 2021. “Correction: A B-Catenin-Driven Switch in TCF/LEF Transcription Factor Binding to DNA Target Sites Promotes Commitment of Mammalian Nephron Progenitor Cells.” ELife 10 (April).
Han, Xiaoping, et al. 2020. “Construction of a Human Cell Landscape at Single-Cell Level.” Nature 581 (7808): 303-9.
Hao, Yuhan, Stephanie Hao, Erica Andersen-Nissen, William M. Mauck III, Shiwei Zheng, Andrew Butler, Maddie J. Lee, et al. 2021. “Integrated Analysis of Multimodal Single-Cell Data.” Cell 184 (13): 3573-3587.e29.
Hao, Yuhan, et al. 2024. “Dictionary Learning for Integrative, Multimodal and Scalable Single-Cell Analysis.” Nature Biotechnology 42 (2): 293-304.
Hatayama, Minoru, and Jun Aruga. 2018. “Role of Zic Family Proteins in Transcriptional Regulation and Chromatin Remodeling.” Advances in Experimental Medicine and Biology 1046:353-80.
Hodge, Rebecca D., et al. 2019. “Conserved Cell Types with Divergent Features in Human versus Mouse Cortex.” Nature 573 (7772): 61-68.
Hupe, Mike, et al. 2017. “Gene Expression Profiles of Brain Endothelial Cells during Embryonic Developmfile:///E:/Documents/Articles/Hupe_gessle_Gene Expression Profiles of Brain Endothelial Cells During_scisignal_2017.Pdfent at Bulk and Single-Cell Levels.” Science Signaling 10 (487): 1-13.
Joung, Julia, Sai Ma, Tristan Tay, Kathryn R. Geiger-Schuller, Paul C. Kirchgatterer, Vanessa K. Verdine, Baolin Guo, et al. 2024. “A Transcription Factor Atlas of Directed Differentiation.” Cell 187 (12): 3161.
Kalucka, Joanna, et al. 2020. “Single-Cell Transcriptome Atlas of Murine Endothelial Cells.” Cell 180 (4): 764-779.e20.
Katt, Moriah E., Zinnia S. Xu, Sharon Gerecht, and Peter C. Searson. 2016. “Human Brain Microvascular Endothelial Cells Derived from the BC1 IPS Cell Line Exhibit a Blood-Brain Barrier Phenotype.” PloS One 11 (4): e0152105.
La Manno, Gioele, Daniel Gyllborg, Simone Codeluppi, Kaneyasu Nishimura, Carmen Salto, Amit Zeisel, Lars E. Borm, et al. 2016. “Molecular Diversity of Midbrain Development in Mouse, Human, and Stem Cells.” Cell 167 (2): 566-580.e19.
Lambert, Samuel A., Arttu Jolma, Laura F. Campitelli, Pratyush K. Das, Yimeng Yin, Mihai Albu, Xiaoting Chen, Jussi Taipale, Timothy R. Hughes, and Matthew T. Weirauch. 2018. “The Human Transcription Factors.” Cell 175 (2): 598-99.
Li, Hui-Hua, Jin Li, Karla J. Wasserloos, Callen Wallace, Mara G. Sullivan, Philip M. Bauer, Donna B. Stolz, et al. 2013. “Caveolae-Dependent and -Independent Uptake of Albumin in Cultured Rodent Pulmonary Endothelial Cells.” PloS One 8 (11): e81903.
Li, Kunpeng, Ya-Jie Dai, Haifeng Zhang, and Zhigang Zhang. 2025. “YAPI Activates SLC2A1 Transcription and Augments the Malignant Behavior of Colorectal Cancer Cells by Activating the Wnt/β-Catenin Signaling Pathway.” Cell Division 20 (1): 8.
Li, Lei, Tao Wan, Min Wan, Bei Liu, Ran Cheng, and Rongying Zhang. 2015. “The Effect of the Size of Fluorescent Dextran on Its Endocytic Pathway.” Cell Biology International 39 (5): 531-39.
Li, Yi-Shuan J., Jason H. Haga, and Shu Chien. 2005. “Molecular Basis of the Effects of Shear Stress on Vascular Endothelial Cells.” Journal of Biomechanics 38 (10): 1949-71.
Lian, X., C. Hsiao, G. Wilson, K. Zhu, L. B. Hazeltine, S. M. Azarin, K. K. Raval, J. Zhang, T. J. Kamp, and S. P. Palecek. 2012. “Cozzarelli Prize Winner: Robust Cardiomyocyte Differentiation from Human Pluripotent Stem Cells via Temporal Modulation of Canonical Wnt Signaling.” Proceedings of the National Academy of Sciences of the United States of America 109 (27): E1848-57.
Liao, Yang, Gordon K. Smyth, and Wei Shi. 2014. “FeatureCounts: An Efficient General Purpose Program for Assigning Sequence Reads to Genomic Features.” Bioinformatics (Oxford, England) 30 (7): 923-30.
Liebner, Stefan, Monica Corada, Thorsten Bangsow, Jane Babbage, Andrea Taddei, Cathrin J. Czupalla, Marco Reis, et al. 2008. “Wnt/Beta-Catenin Signaling Controls Development of the Blood-Brain Barrier.” The Journal of Cell Biology 183 (3): 409-17.
Lippmann, Ethan S., Abraham Al-Ahmad, Samira M. Azarin, Sean P. Palecek, and Eric V. Shusta. 2014. “A Retinoic Acid-Enhanced, Multicellular Human Blood-Brain Barrier Model Derived from Stem Cell Sources.” Scientific Reports 4 (February): 4160.
Lippmann, Ethan S., Samira M. Azarin, Jennifer E. Kay, Randy A. Nessler, Hannah K. Wilson, Abraham Al-Ahmad, Sean P. Palecek, and Eric V. Shusta. 2012. “Derivation of Blood-Brain Barrier Endothelial Cells from Human Pluripotent Stem Cells.” Nature Biotechnology 30 (8): 783-91.
Lu, Tyler M., et al. 2021. “Pluripotent Stem Cell-Derived Epithelium Misidentified as Brain Microvascular Endothelium Requires ETS Factors to Acquire Vascular Fate.” Proceedings of the National Academy of Sciences of the United States of America 118 (8): e2016950118.
MacDonald, Bryan T., Keiko Tamai, and Xi He. 2009. “Wnt/Beta-Catenin Signaling: Components, Mechanisms, and Diseases.” Developmental Cell 17 (1): 9-26.
Mansisidor, Andrés R., and Viviana I. Risca. 2022. “Chromatin Accessibility: Methods, Mechanisms, and Biological Insights.” Nucleus (Austin, Tex.) 13 (1): 236-76.
Martinez-Corral, Rosa, et al. 2023. “Transcriptional Kinetic Synergy: A Complex Landscape Revealed by Integrating Modeling and Synthetic Biology.” Cell Systems 14 (4): 324-339.e7.
Meulen, Talitha van der, and Mark O. Huising. 2015. “Role of Transcription Factors in the Transdifferentiation of Pancreatic Islet Cells.” Journal of Molecular Endocrinology 54 (2): R103-17.
Moonen, Jan-Renier, et al. 2022. “KLF4 Recruits SWI/SNF to Increase Chromatin Accessibility and Reprogram the Endothelial Enhancer Landscape under Laminar Shear Stress.” Nature Communications 13 (1): 4941.
Ng, Alex H. M., et al. 2020. “A Comprehensive Library of Human Transcription Factors for Cell Fate Engineering.” Nature Biotechnology, November.
Ohshima, Makiko, et al. 2019. “Prediction of Drug Permeability Using in Vitro Blood-Brain Barrier Models with Human Induced Pluripotent Stem Cell-Derived Brain Microvascular Endothelial Cells.” BioResearch Open Access 8 (1): 200-209.
Paudel, Rupesh, Lorenza Fusi, and Marc Schmidt. 2021. “The MEK5/ERK5 Pathway in Health and Disease.” International Journal of Molecular Sciences 22 (14): 7594.
Pavlacky, Jiri, and Jan Polak. 2020. “Technical Feasibility and Physiological Relevance of Hypoxic Cell Culture Models.” Frontiers in Endocrinology 11 (February): 57.
Pawlowski, Matthias, et al. 2017. “Inducible and Deterministic Forward Programming of Human Pluripotent Stem Cells into Neurons, Skeletal Myocytes, and Oligodendrocytes.” Stem Cell Reports 8 (4): 803-12.
Polioudakis, Damon, L, et al. 2019. “A Single-Cell Transcriptomic Atlas of Human Neocortical Development during Mid-Gestation.” Neuron 103 (5): 785-801.e8.
Pollak, Nina M., Matthew Hoffman, Ira J. Goldberg, and Konstantinos Drosatos. 2018. “Krüppel-like Factors.” JACC. Basic to Translational Science 3 (1): 132-56.
Porkoláb, Gergő, et al. 2024. “Synergistic Induction of Blood-Brain Barrier Properties.” Proceedings of the National Academy of Sciences of the United States of America 121 (21): e2316006121.
Ream, Michael W., et al. 2024. “Direct Programming of Human Pluripotent Stem Cells into Endothelial Progenitors with SOX17 and FGF2.” Stem Cell Reports 19 (4): 579-95.
Roudnicky, Filip, et al. 2020. “Inducers of the Endothelial Cell Barrier Identified through Chemogenomic Screening in Genome-Edited HPSC-Endothelial Cells.” Proceedings of the National Academy of Sciences of the United States of America 117 (33): 19854-65.
Rubenstein, Aliza B., et al. 2020. “Single-Cell Transcriptional Profiles in Human Skeletal Muscle.” Scientific Reports 10 (1): 229.
Satija, Rahul, et al. 2015. “Spatial Reconstruction of Single-Cell Gene Expression Data.” Nature Biotechnology 33 (5): 495-502.
Seitz, Roswitha, et al. 2010. “Norrin Mediates Neuroprotective Effects on Retinal Ganglion Cells via Activation of the Wnt/β-Catenin Signaling Pathway and the Induction of Neuroprotective Growth Factors in Müller Cells.” The Journal of Neuroscience: The Official Journal of the Society for Neuroscience 30 (17): 5998-6010.
Sharma, Divya, et al. 2021. “A Review of the Tortuous Path of Nonviral Gene Delivery and Recent Progress.” International Journal of Biological Macromolecules 183 (July): 2055-73.
Simitian, Grigor, et al. 2022. “Microfluidics in Vascular Biology Research: A Critical Review for Engineers, Biologists, and Clinicians.” Lab on a Chip 22 (19): 3618-36.
Stamos, Jennifer L., and William I. Weis. 2013. “The β-Catenin Destruction Complex.” Cold Spring Harbor Perspectives in Biology 5 (1): a007898.
Stebbins, Matthew J., et al. 2018. “Activation of RARα, RARγ, or RXRα Increases Barrier Tightness in Human Induced Pluripotent Stem Cell-Derived Brain Endothelial Cells.” Biotechnology Journal 13 (2): 1-12.
Stuart, Tim, et al. 2019. “Comprehensive Integration of Single-Cell Data.” Cell 177 (7): 1888-1902.e21.
Sun, Na, et al. 2023. “Single-Nucleus Multiregion Transcriptomic Analysis of Brain Vasculature in Alzheimer's Disease.” Nature Neuroscience 26 (6): 970-82.
Sweet, David R., Liyan Fan, Paishiun N. Hsieh, and Mukesh K. Jain. 2018. “Krüppel-like Factors in Vascular Inflammation: Mechanistic Insights and Therapeutic Potential.” Frontiers in Cardiovascular Medicine 5 (February): 6.
Sweet, David R., et al. 2023. “Krüppel-like Factors Orchestrate Endothelial Gene Expression through Redundant and Non-redundant Enhancer Networks.” Journal of the American Heart Association 12 (4): e024303.
Takahashi, Kazutoshi, and Shinya Yamanaka. 2016. “A Decade of Transcription Factor-Mediated Reprogramming to Pluripotency.” Nature Reviews. Molecular Cell Biology 17 (3): 183-93.
Tomaz, Rute A., et al. 2022. “Generation of Functional Hepatocytes by Forward Programming with Nuclear Receptors.” ELife 11 (August).
Travaglini, Kyle J., et al. 2020. “A Molecular Cell Atlas of the Human Lung from Single-Cell RNA Sequencing.” Nature 587 (7835): 619-25.
Tsaryk, Roman, et al. 2022. “Shear Stress Switches the Association of Endothelial Enhancers from ETV/ETS to KLF Transcription Factor Binding Sites.” Scientific Reports 12 (1): 4795.
Van Rossum, Guido, and Python Development Team. 2018. The Python Library Reference. Suwanee, GA: 12th Media Services.
Vatine, Gad D., et al. 2019. “Human IPSC-Derived Blood-Brain Barrier Chips Enable Disease Modeling and Personalized Medicine Applications.” Cell Stem Cell 24 (6): 995-1005.e6.
Wang, Huan, Xiao Li, Shutao Gao, Xuying Sun, and Huang Fang. 2015. “Transdifferentiation via Transcription Factors or MicroRNAs: Current Status and Perspective.” Differentiation; Research in Biological Diversity 90 (4-5): 69-76.
Wang, Kai, et al. 2020. “Robust Differentiation of Human Pluripotent Stem Cells into Endothelial Cells via Temporal Modulation of ETV2 with Modified MRNA.” Science Advances 6 (30): eaba7606.
Wang, Zhongxiao, et al. 2022. “Amino Acid Transporter SLC38A5 Regulates Developmental and Pathological Retinal Angiogenesis.” ELife 11 (December).
Waskom, Michael. 2021. “Seaborn: Statistical Data Visualization.” Journal of Open Source Software 6 (60): 3021.
Watanabe, Hitoshi, et al. 2014. “HHEX Promotes Hepatic-Lineage Specification through the Negative Regulation of Eomesodermin.” PloS One 9 (3): e90791.
Winkler, Ethan A., et al. 2022. “A Single-Cell Atlas of the Normal and Malformed Human Brain Vasculature.” Science (New York, N.Y.) 375 (6584): eabi7377.
Yang, Andrew C., et al. 2022. “A Human Brain Vascular Atlas Reveals Diverse Mediators of Alzheimer's Risk.” Nature 603 (7903): 885-92.
Zarkada, Georgia, et al. 2021. “Specialized Endothelial Tip Cells Guide Neuroretina Vascularization and Blood-Retina-Barrier Formation.” Developmental Cell 56 (15): 2237-2251.e6.
Zhao, Yong-Lin, et al. 2014. “Role of Caveolin-1 in the Biology of the Blood-Brain Barrier.” Reviews in the Neurosciences 25 (2): 247-54.
Zhong, Suijuan, et al. 2020. “Decoding the Development of the Human Hippocampus.” Nature 577 (7791): 531-36.

We claim:

1. A method for producing an endothelial cell capable of forming a confluent monolayer with blood-brain barrier (BBB)-like properties, the method comprising:
(a) culturing an endothelial cell or an endothelial progenitor cell in a medium comprising a Wnt/β-catenin signaling activator, and
(b) expressing one or more transcription factors in the endothelial progenitor cells for at least two days, wherein the one or more transcription factors are selected from the group consisting of DACH1, DACH2, FLI1, FOS, FOXC1, FOXF1, FOXF2, FOXQ1, HES1, JUN, KLF2, KLF4, LEF1, MECOM, NR4A1, NR4A2, PPARD, TBX3, TSC22D1, ZIC2, ZIC3 and combinations thereof.

2. The method of claim 1, wherein the BBB-like properties comprise increased tight junction protein expression, increased transporter protein expression, reduced transcytosis-related protein expression, reduced leukocyte adhesion molecule expression and combinations thereof as compared to endothelial progenitor cells not expressing the transcription factors.

3. The method of claim 1, wherein the endothelial cells have increased expression of one or more proteins comprising OCLN, CLDN5, MFSD2A, SLC2A1, SLC38A5, SLC7A5, ABCB1, ABCG2 and combinations thereof.

4. The method of claim 1, wherein the endothelial cells have decreased expression of one or more proteins comprising PLVAP, CAV1, CAV2, ICAM1 and combinations thereof.

5. The method of claim 1, wherein the Wnt/β-catenin signaling activator is CHIR99021.

6. The method of claim 5, wherein the CHIR99021 in the culture is at a concentration of 3-5 μM.

7. The method of claim 1, wherein the endothelial progenitor cell was differentiated from a pluripotent stem cell.

8. The method of claim 1, wherein the one or more transcription factors are overexpressed.

9. The method of claim 8, wherein overexpression of the one or more transcription factors is achieved via transduction with a virus comprising a polynucleotide encoding one or more transcription factors operably linked to a promoter function in the endothelial progenitor cell.

10. The method of claim 1, wherein two or more transcription factors are selected from the group consisting of KLF2, KLF4, ZIC2, ZIC3, NR4A1, NR4A2, FOXQ1, FOXF1 and FOXF2 and combinations thereof.

11. The method of any one of claim 10, wherein the two or more transcription factors comprise FOXF1 or FOXF2, ZIC3 or ZIC2, NR4A1 or NR4A2, FOXQ1, and KLF2 or KLF4.

12. The method of claim 10, wherein the two or more transcription factors are selected from the groups consisting of:
(a) KLF2 or KLF4, FOX F1 or FOXF2, NR4A1 or NR4A2, and FOXQ1; or
(b) KLF2 or KLF4, FOXF1 or FOXF2, ZIC2 or ZIC3, and FOXQ1; or
(c) KLF2 or KLF4, NR4A1 or NR4A2, ZIC2 or ZIC3 and FOXQ1; or
(d) KLF2 or KLF4, FOXF1 or FOXF2, NR4A1 or NR4A2, and ZIC2 or ZIC3; or
(e) FOXF1 or FOXF2, FOXQ1, NR4A1 or NR4A2, and ZIC2 or ZIC3; or
(f) KLF2 or KLF4, FOXF1 or FOXF2, ZIC2 or ZIC3, NR4A1 or NR4A2, and FOXQ1; or
(g) KLF2, FOXF1, ZIC3, NR4A2, and FOXQ1.

13. The method of claim 1, wherein the transcription factors consist of DACH1, DACH2, FLI1, FOS, FOXC1, FOXF1, FOXF2, FOXQ1, HES1, JUN, KLF2, KLF4, LEF1, MECOM, NR4A1, NR4A2, PPARD, TBX3, TSC22D1, ZIC2 and ZIC3.

14. A population of endothelial cells with BBB-like properties produced by the method of claim 1.

15. An in vitro BBB model comprising a confluent monolayer of the endothelial cells of claim 14 cultured on a surface, wherein the BBB model has BBB-like properties.

16. The BBB model of claim 15, wherein the endothelial cells are derived from pluripotent stem cells obtained from a subject.

17. The BBB model of claim 16, wherein the endothelial cells comprise a mutation associated with a disease.

18. The BBB model of claim 16, wherein the subject has a brain disease.

19. A method of using the BBB model of claim 15, comprising contacting the BBB model with a therapeutic agent and testing the ability of the therapeutic agent to cross the BBB.

20. The method of claim 19, wherein the therapeutic agent comprises small molecule drugs, biologics, viral vectors, or therapeutic cells.
